(19) United States
(12) Patent Application Publication
(10) Pub. No.: US 2026/0201069 A1
(43) Pub. Date: Jul. 16, 2026

(54) ANTIBODIES THAT SPECIFICALLY BIND MODIFIED OLIGONUCLEOTIDES

(71) Applicant: Hoffmann-La Roche Inc., Little Falls, NJ (US)

(72) Inventors: Joerg BENZ, Rheinfelden (DE); Ulrich BRINKMANN, Weilheim (DE); Thomas EMRICH, Iffeldorf (DE); Guy GEORGES, Habach (DE); Hung-En HSIA, München (DE)

(73) Assignee: Hoffmann-La Roche Inc., Little Falls, NJ (US)

(21) Appl. No.: 19/176,867

(22) Filed: Apr. 11, 2025

(30) Foreign Application Priority Data
Apr. 11, 2024 (EP) 24169668.1
Dec. 4, 2024 (EP) 24217361.5

(51) Int. Cl. C07K 16/44

(52) U.S. Cl. CPC C07K 16/44; C07K 2317/31

(57) ABSTRACT

Herein is reported an anti-LNA antibody comprising six HVRs of SEQ ID NO: 42, 44, 46, 51, 53 and 55.

### CROSS-REFERENCE TO RELATED APPLICATIONS

This application claims benefit of priority to European Application No. 24217361.5, filed Dec. 4, 2024, and claims benefit to European Application No. 24169668.1, filed Apr. 11, 2024, each of which is incorporated herein by reference in its entirety.

### SEQUENCE LISTING

The instant application contains a sequence listing which has been submitted electronically in .xml format and is hereby incorporated by reference in its entirety.

Said .xml copy, created on Apr. 9, 2025, is named P39300-US_Sequence_Listing.xml and is 181,919 bytes in size and updated by a file titled P39300-US_Replacement_Sequence_Listing.xml created on Nov. 4, 2025, which is 187,787 bytes in size.

### FIELD OF THE INVENTION

The current invention is in the field of antibody technology. In more detail herein are reported antibodies that specifically bind modified oligonucleotides (LNAs), their generation, characterization and use, for example, in analytics and antibody-targeted LNA-delivery.

### BACKGROUND OF THE INVENTION

Antisense oligonucleotides (ASO) are modified nucleic acids that elicit biological functionality by inhibiting the activity of the products of target genes defined by sequence complementarity. One subtype of ASOs frequently applied for silencing or RNA interference of target gene activity are locked nucleic acid analogues (LNA) which harbor nucleotides with a bicyclic furanose unit locked in an RNA mimicking sugar conformation. Such LNAs frequently contain additionally phosphorothioate instead of phosphate bridges between individual nucleotides. LNAs with optimized nucleic acid modifications display beneficial properties related to stability, cellular uptake and efficacy when compared to oligonucleotides without such modifications.

However, those modifications can also alter the biophysical properties of oligonucleotides in an undesired manner. This can affect solubility, PK-properties, non-specific attachment to cell surfaces, interference with specific targeting approaches, and confer developability challenges.

Furthermore, the presence of unnatural nucleic acid derivatives can cause therapeutic LNAs to become recognized by the immune system, leading to the development of anti-drug antibodies (ADA). The assessment of potential ADAs is therefore required for the clinical development of therapeutic LNAs.

Thus, there is a need for means for addressing these issues.

### SUMMARY OF THE INVENTION

Herein are reported monoclonal antibodies that specifically bind single-stranded LNAs. In more detail, the antibodies according to the current invention specifically bind to LNAs that harbor specific modifications, but do not bind unmodified single- or double-stranded nucleic acids.

With the antibodies according to the current invention, amongst other things, the cloaking of modules to counteract or ameliorate potential issues of LNAs and LNA-conjugates becomes possible.

With the antibodies according to the current invention, amongst other things, immunoassays can be provided that allow the detection of LNA-specific ADAs and that allow the determination of the pharmacokinetic (PK) properties of ASO-containing compounds.

In more detail, the current invention encompasses at least the following embodiments
1. An anti-LNA antibody comprising
   (a) HVR-H1 comprising the amino acid sequence of SEQ ID NO: 42;
   (b) HVR-H2 comprising the amino acid sequence of SEQ ID NO: 44;
   (c) HVR-H3 comprising the amino acid sequence of SEQ ID NO: 46;
   (d) HVR-L1 comprising the amino acid sequence of SEQ ID NO: 51;
   (e) HVR-L2 comprising the amino acid sequence of SEQ ID NO: 53; and
   (f) HVR-L3 comprising the amino acid sequence of SEQ ID NO: 55.
2. The anti-LNA antibody according to embodiment 1, wherein the antibody is a chimeric or humanized antibody.
3. The anti-LNA antibody according to embodiment 1 comprising a VH of SEQ ID NO: 48 and a VL of SEQ ID NO: 57.
4. The anti-LNA antibody according to any one of embodiments 1 to 3, wherein the antibody is an antibody fragment selected from the group of antibody fragments consisting of Fv, scFv, Fab and scFab.
5. The anti-LNA antibody according to any one of embodiments 1 to 3, wherein the antibody comprises
   a) a full length constant region of the human subclass IgG1, or
   b) a full length constant region of the human subclass IgG4, or
   c) a full length constant region of the human subclass IgG1 with the mutations L234A, L235A and P329G (numbering according to Kabat EU index),
   d) a full length constant region of the human subclass IgG4 with the mutations S228P and L235E (numbering according to Kabat EU index),
   e) a full length constant region of the human subclass IgG1 with the mutations L234A, L235A and P329G in both heavy chains and the mutation T366W in one heavy chain and the mutations T366S, L368A and Y407V in the respective other heavy chain (numbering according to Kabat EU index),
   f) a full length constant region of the human subclass IgG1 with the mutations L234A, L235A and P329G in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
   g) a full length constant region of the human subclass IgG4 with the mutations S228P and L235E in both heavy chains and the mutation T366W in one heavy chain and the mutations T366S, L368A and Y407V in the respective other heavy chain (numbering according to Kabat EU index),
   h) a full length constant region of the human subclass IgG4 with the mutations S228P and L235E in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
   i) a full length constant region of the human subclass IgG1 with the mutations L234A, L235A, P329G, I253A, H310A and H435A in both heavy chains and the mutation T366W in one heavy chain and the mutations T366S, L368A and Y407V in the respective other heavy chain (numbering according to Kabat EU index),
   j) a full length constant region of the human subclass IgG1 with the mutations L234A, L235A, P329G, I253A, H310A and H435A in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
   k) a full length constant region of the human subclass IgG1 with the mutations L234A, L235A, P329G, M252Y, S254T and T256E in both heavy chains and the mutation T366W in one heavy chain and the mutations T366S, L368A and Y407V in the respective other heavy chain (numbering according to Kabat EU index),
   l) a full length constant region of the human subclass IgG1 with the mutations L234A, L235A, P329G, M252Y, S254T and T256E in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
   m) a full length constant region of the human subclass IgG1 with the mutations L234A, L235A, P329G, H310A, H433A and Y436A in both heavy chains and the mutation T366W in one heavy chain and the mutations T366S, L368A and Y407V in the respective other heavy chain (numbering according to Kabat EU index), or
   n) a full length constant region of the human subclass IgG1 with the mutations L234A, L235A, P329G, H310A, H433A and Y436A in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index).
5a. A multispecific antibody comprising
   at least one first binding site specifically binding to LNA and comprising
      (a) HVR-H1 comprising the amino acid sequence of SEQ ID NO: 42;
      (b) HVR-H2 comprising the amino acid sequence of SEQ ID NO: 44;
      (c) HVR-H3 comprising the amino acid sequence of SEQ ID NO: 46;
      (d) HVR-L1 comprising the amino acid sequence of SEQ ID NO: 51;
      (e) HVR-L2 comprising the amino acid sequence of SEQ ID NO: 53; and
      (f) HVR-L3 comprising the amino acid sequence of SEQ ID NO: 55;
   at least one second binding site specifically binding to a second non-LNA target,
   an Fc-region comprising a first Fc-region polypeptide and a second Fc-region polypeptide,
   wherein the at least one binding site specifically binding to LNA is conjugated to the C-terminus of one of the Fc-region polypeptides and the at least one binding site specifically binding to a second non-LNA target is conjugated to the N-terminus of one of the Fc-region polypeptides.
6. The multispecific antibody according to embodiment 5a, wherein the antibody comprises
   a) one first binding site and one second binding site, whereby both binding sites are conjugated to the same Fc-region polypeptide;
   b) one first binding site and one second binding site, whereby both binding sites are conjugated to different Fc-region polypeptides;
   c) two first binding sites each comprising the HVRs of SEQ ID NO: 42, 44, 46, 51, 53 and 55, one second binding site, whereby the C-terminus of each Fc-region polypeptide is conjugated to a single first binding site; or
   d) two first binding sites each comprising the HVRs of SEQ ID NO: 42, 44, 46, 51, 53 and 55, and two second binding sites, whereby the N-terminus of each Fc-region polypeptide is conjugated to a single second binding site and the C-terminus of each Fc-region polypeptide is conjugated to a single first binding site.
7. The multispecific antibody according to any one of embodiments 5a to 6, wherein each binding site is independently of each other an antibody fragment selected from the group of antibody fragments consisting of Fv, scFv, Fab and scFab.
8. The multispecific antibody according to any one of embodiments 5a to 7, wherein the at least one first binding site or the at least one second binding site of the at least one first and second binding site is a chimeric or humanized binding site.
9. The multispecific antibody according to any one of embodiments 5a to 8, wherein the at least one first binding site comprises a VH of SEQ ID NO: 48 and a VL of SEQ ID NO: 57.
10. The multispecific antibody according to any one of embodiments 5a to 9, wherein the at least one first binding site is conjugated to the C-terminus of the respective Fc-region polypeptide by a peptidic linker.
11. The multispecific antibody according to embodiment 10, wherein the peptidic linker is a GS-linker comprising GGGS (SEQ ID NO: 148) or GGGGS (SEQ ID NO: 149) elements and a total number of amino acid residues in the range of and including 20 amino acid residues to 40 amino acid residues.
12. The multispecific antibody according to any one of embodiments 5a to 11, wherein the Fc-region comprises
   a) a first and a second Fc-region polypeptide each of the human subclass IgG1, or
   b) a first and a second Fc-region polypeptide each of the human subclass IgG4, or
   c) a first and a second Fc-region polypeptide each of the human subclass IgG1 each with the mutations L234A, L235A and P329G (numbering according to Kabat EU index),
   d) a first and a second Fc-region polypeptide each of the human subclass IgG4 with the mutations S228P and L235E (numbering according to Kabat EU index),
   e) a first and a second Fc-region polypeptide each of the human subclass IgG1 with the mutations L234A, L235A and P329G and the mutation T366W in one Fc-region polypeptide and the mutations T366S, L368A and Y407V in the respective other Fc-region polypeptide (numbering according to Kabat EU index),
   f) a first and a second Fc-region polypeptide each of the human subclass IgG1 with the mutations L234A, L235A and P329G and the mutations i) T366W, and ii) S354C or Y349C, in one Fc-region polypeptide and the mutations i) T366S, L368A, and Y407V, and ii) Y349C or S354C, in the respective other Fc-region polypeptide (numbering according to Kabat EU index),
   g) a first and a second Fc-region polypeptide each of the human subclass IgG4 with the mutations S228P and L235E and the mutation T366W in one Fc-region polypeptide and the mutations T366S, L368A and Y407V in the respective other Fc-region polypeptide (numbering according to Kabat EU index),
   h) a first and a second Fc-region polypeptide each of the human subclass IgG4 with the mutations S228P and L235E and the mutations i) T366W, and ii) S354C or Y349C, in one Fc-region polypeptide and the mutations i) T366S, L368A, and Y407V, and ii) Y349C or S354C, in the respective other Fc-region polypeptide (numbering according to Kabat EU index),
   i) a first and a second Fc-region polypeptide each of the human subclass IgG1 with the mutations L234A, L235A, P329G, I253A, H310A and H435A and the mutation T366W in one Fc-region polypeptide and the mutations T366S, L368A and Y407V in the respective other Fc-region polypeptide (numbering according to Kabat EU index),
   j) a first and a second Fc-region polypeptide each of the human subclass IgG1 with the mutations L234A, L235A, P329G, I253A, H310A and H435A and the mutations i) T366W, and ii) S354C or Y349C, in one Fc-region polypeptide and the mutations i) T366S, L368A, and Y407V, and ii) Y349C or S354C, in the respective other Fc-region polypeptide (numbering according to Kabat EU index),
   k) a first and a second Fc-region polypeptide each of the human subclass IgG1 with the mutations L234A, L235A, P329G, M252Y, S254T and T256E and the mutation T366W in one Fc-region polypeptide and the mutations T366S, L368A and Y407V in the respective other Fc-region polypeptide (numbering according to Kabat EU index),
   l) a first and a second Fc-region polypeptide each of the human subclass IgG1 with the mutations L234A, L235A, P329G, M252Y, S254T and T256E and the mutations i) T366W, and ii) S354C or Y349C, in one Fc-region polypeptide and the mutations i) T366S, L368A, and Y407V, and ii) Y349C or S354C, in the respective other Fc-region polypeptide (numbering according to Kabat EU index),
   m) a first and a second Fc-region polypeptide each of the human subclass IgG1 with the mutations L234A, L235A, P329G, H310A, H433A and Y436A and the mutation T366W in one Fc-region polypeptide and the mutations T366S, L368A and Y407V in the respective other Fc-region polypeptide (numbering according to Kabat EU index), or
   n) a first and a second Fc-region polypeptide each of the human subclass IgG1 with the mutations L234A, L235A, P329G, H310A, H433A and Y436A and the mutations i) T366W, and ii) S354C or Y349C, in one Fc-region polypeptide and the mutations i) T366S, L368A, and Y407V, and ii) Y349C or S354C, in the respective other Fc-region polypeptide (numbering according to Kabat EU index).
13. The anti-LNA antibody according to any one of embodiments 1 to 4 or the multispecific antibody according to any one of embodiments 5a to 12, wherein the antibody comprises
   an Fc-region comprising a first Fc-region polypeptide and a second Fc-region polypeptide, and
   at least one recognition site(s) for the transglutaminase from Kutzneria albida (KalbTG) inserted in one or both of the Fc-region polypeptides.
14. The anti-LNA antibody or the multispecific antibody according to embodiment 13, wherein the at least one recognition site is inserted directly after one of the positions from the group of positions consisting of position 297, position 341 and position 401 of the heavy chain (numbering according to Kabat), in case of more than one recognition site the positions are selected independently of each other.
15. The anti-LNA antibody or the multispecific antibody according to embodiment 13, wherein either one recognition site or one recognition site in each Fc-region polypeptide is(are) inserted directly after position 297 of the Fc-region polypeptide (numbering according to Kabat).
16. The anti-LNA antibody or the multispecific antibody according to any one of embodiments 13 to 15, wherein each of the at least one recognition sites has the sequence of SEQ ID NO: 134 or of SEQ ID NO: 143, in case of more than one recognition site independently of each other.
17. The anti-LNA antibody or the multispecific antibody according to any one of embodiments 13 to 16, wherein the antibody is conjugated to a payload via the at least one recognition site using KTG.
18. The anti-LNA antibody or the multispecific antibody according to embodiment 17, wherein the payload is selected from a small molecule, a peptide or polypeptide, a dye, a nucleic acid, an siRNA, an antisense oligonucleotide and an LNA.
20. A pharmaceutical composition comprising the anti-LNA antibody according to any one of embodiments 1 to 4 and 13 to 18 or the multispecific antibody according to any one of embodiments 5a to 18.
21. The anti-LNA antibody according to any one of embodiments 1 to 4 and 13 to 18 or the multispecific antibody according to any one of embodiments 5a to 18 for use as a medicament.
22. The use of the anti-LNA antibody according to any one of embodiments 1 to 4 and 13 to 18 or the multispecific antibody according to any one of embodiments 5a to 18 for the manufacture of a medicament.
23. A nucleic acid or a composition of nucleic acids encoding the anti-LNA antibody according to any one of embodiments 1 to 4 and 13 to 16 or the multispecific antibody according to any one of embodiments 5a to 16.
24. A cell comprising the nucleic acid or the composition of nucleic acids according to embodiment 23.
25. A method for producing the anti-LNA antibody according to any one of embodiments 1 to 4 and 13 to 16 or the multispecific antibody according to any one of embodiments 5a to 16, wherein the method comprises the following steps:
   cultivating a cell according to embodiment 24 in a cultivation medium under conditions suitable for the expression of the antibody,
   recovering the antibody from the cell or the cultivation medium, and
   optionally purifying the antibody with at least one chromatography step.
26. A method for producing the anti-LNA antibody or the multispecific antibody according to any one of embodiments 17 to 18, wherein the method comprises the following steps:
   cultivating a cell according to embodiment 24 in a cultivation medium under conditions suitable for the expression of the antibody,
   recovering the antibody from the cell or the cultivation medium,
   optionally purifying the antibody with at least one chromatography step,
   conjugating the antibody with a payload using KTG to obtain a conjugate, and
   optionally purifying the conjugate with at least one chromatography step.

In addition to the various embodiments depicted and claimed, the disclosed subject matter is also directed to other embodiments having other combinations of the features disclosed or claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DESCRIPTION OF THE FIGURES

FIG. 1 Epitope-mapping results of the antibody produced by clone 1.2.8.

FIG. 2 Epitope-mapping results of the antibody produced by clone 1.9.21.

FIG. 3 Epitope-mapping results of the antibody produced by clone 1.4.10.

FIG. 4 Epitope-mapping results of the antibody produced by clone 1.5.13.

FIG. 5 Epitope-mapping results of the antibody produced by clone 1.7.15.

FIG. 6 Binding of bivalent anti-LNA antibody according to the current invention produced by clone 1.2.8 to the four different LNA-modified ASOs as depicted in Table 3-2.

FIG. 7 Binding of bivalent anti-LNA antibody according to the current invention produced by clone 1.7.15 to the four different LNA-modified ASOs as depicted in Table 3-2.

FIG. 8 Binding of bivalent anti-LNA antibody according to the current invention produced by clone 1.9.21 to the four different LNA-modified ASOs as depicted in Table 3-2.

FIG. 9 Binding of monovalent anti-LNA antibody according to the current invention produced by clone 1.9.21 to the LNA-modified ASO 420 as depicted in Table 3-3.

FIG. 10 Binding of monovalent anti-LNA antibody according to the current invention produced by clone 1.9.21 to the LNA-modified ASO 827 as depicted in Table 3-3.

FIG. 11 Binding of monovalent anti-LNA antibody according to the current invention produced by clone 1.9.21 to the LNA-modified ASO 297 as depicted in Table 3-3.

FIG. 12 Binding of monovalent anti-LNA antibody according to the current invention produced by clone 1.9.21 to the LNA-modified ASO 042 as depicted in Table 3-3.

FIG. 13 Binding of monovalent anti-LNA antibody according to the current invention produced by clone 1.9.21 to the siRNA 664 as depicted in Table 3-3.

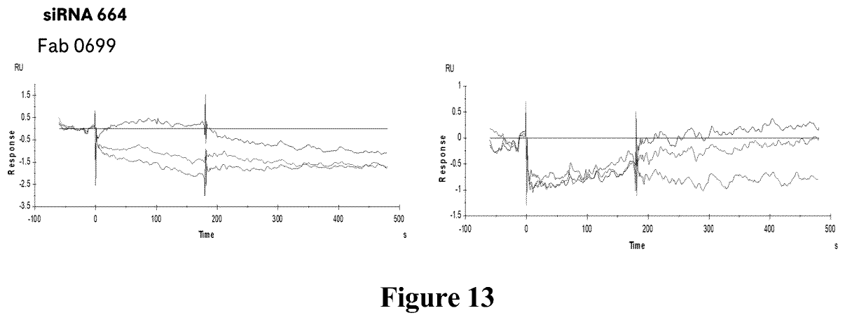

FIG. 14 Scheme of a bridging ADA assay; detection from a sample (ADA) and positive control using an antibody according to the current invention (ADA PC).

FIG. 15 Scheme of a direct ADA assay; detection from a sample (ADA) and positive control using an antibody according to the current invention with a species-specific Fc- or constant region (ADA PC).

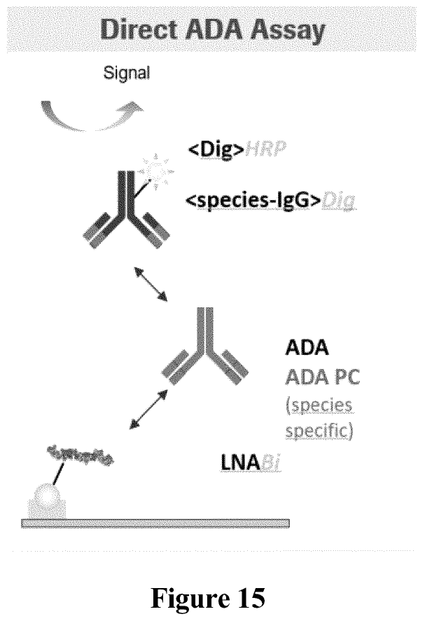

FIG. 16 Calibration curve of a direct ADA assay with an antibody according to the current invention as calibrator/positive control.

FIG. 17 Serial dilution of GalNAc-conjugated LNA (3.8 to 60 ng/mL plasma concentration) and quantitative detection thereof in a generic LNA immunoassay.

FIG. 18 Example of standard curves obtained with three different antibody-ASO conjugates using an antibody according to the current invention as capture antibody.

FIG. 19 Crystal structure of Fab 0699 with ASO 980. View onto the binding site of ASO 980 bound to Fab 0699. ASO 980 is colored in salmon, the light and heavy chain of Fab 0699 are colored in cyan and blue, respectively. A HEPES buffer molecule from the crystallization buffer bound to the Fab is depicted in yellow.

FIG. 20 Sketch of the structure of bispecific anti-TfR/LNA antibody conjugated to an LNA-modified ASO.

FIG. 21 SEC chromatogram of a sample comprising a bispecific anti-TfR/LNA antibodies conjugated to an LNA-modified ASO.

FIG. 22 Sketch of the structure of monospecific anti-TfR antibodies conjugated to an LNA-modified ASO.

FIG. 23 Sketches of different bispecific anti-germline/LNA antibodies and anti-TfR/LNA antibodies conjugated to an LNA-modified ASO 576.

FIG. 24 SEC chromatograms of the produced antibodies of FIG. 23.

FIG. 25 Sketches of the bispecific anti-TfR/LNA antibodies conjugated to an LNA-modified ASO 576 with 20 amino acid peptidic GS linker as produced with enzymatic conjugation followed by click chemistry conjugation.

FIG. 26 SEC chromatograms of the produced antibodies of FIG. 25.

FIG. 27 Object-based colocalization analysis between IgG and transferrin receptor of different antibodies and complexes incubated with hCMED/D3 cells (mAb 3732; fusion 2489; fab 1988; fab 0699).

FIG. 28 Object-based colocalization analysis of IgG with transferrin receptor using FORCE-generated bispecific anti-TfR/LNA and anti-DP47/LNA Antibodies conjugated to LNA-modified ASO payloads.

FIG. 29 Object-based colocalization analysis of IgG with transferrin receptor using bispecific anti-TfR/LNA and anti-DP47/LNA Antibodies conjugated to LNA-modified ASO payloads generated with conventional recombinant expression method.

FIG. 30 Intracellular mean intensity of LNA-modified ASO from a non-covalent complex of a bispecific anti-TfR/LNA antibody and an LNA-modified ASO payload.

FIG. 31 Intracellular mean intensity of IgG from a non-covalent complex of a bispecific anti-TfR/LNA antibody and an LNA-modified ASO payload.

FIG. 32 Object-based colocalization analysis between IgG and transferrin receptor of a non-covalent complex of antibody and an LNA-modified ASO payload.

FIG. 33 Fluorescence intensity in 3D-BBB spheroids.

FIG. 34 Fluorescence intensity in 3D-BBB spheroids.

FIG. 35 Florescence intensity in 3D-BBB spheroids.

FIG. 36 LUCA results.

FIG. 37 LUCA results.

FIG. 38 LUCA results.

FIG. 39 ASO plasma PK in mice using antibody-ASO complex (non-conjugates).

FIG. 40 ASO plasma PK in mice using Antibody-ASO conjugates.

FIG. 41 ASO levels in the Cortex brain region of mice.

FIG. 42 ASO levels in the Cerebellum brain region of mice.

FIG. 43 ASO levels in non-Cortex and non-Cerebellum brain regions of mice.

### DETAILED DESCRIPTION OF THE INVENTION

The current invention is directed to monoclonal antibodies that specifically bind single-stranded LNAs. In more detail, the antibodies according to the current invention specifically bind to LNAs that harbor specific modifications, but do not bind unmodified single- or double-stranded nucleic acids.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

Likewise the hypervariable regions (HVRs) in the heavy and light chain variable domains of non-human and human antibodies are determined following Kabat, E. A., et al., Sequences of Proteins of Immunological Interest, 5th ed., Public Health Service, National Institutes of Health, Bethesda, MD (1991). Accordingly, the HVRs of the antibodies according to the current invention have been determined according to Kabat and, thus, are denoted as “according to Kabat”.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987). The content of which is incorporated herein by reference,

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless otherwise defined herein the term “comprising of” shall include the term “consisting of”.

The term “about” as used herein in connection with a specific value (e.g. temperature, concentration, time and others) shall refer to a variation of +/−1% of the specific value that the term “about” refers to.

The terms “expression” and “expresses” are used herein to refer to transcription and translation occurring within a cell. The level of expression of a nucleic acid in a cell can be determined on the basis of either the amount of corresponding mRNA that is present in the cell or the amount of the protein encoded by the nucleic acid that is produced by the cell. For example, mRNA transcribed from a nucleic acid is desirably quantitated by northern hybridization. Sambrook et al., Molecular Cloning: A Laboratory Manual, pp. 7.3-7.57 (Cold Spring Harbor Laboratory Press, 1989).

Protein encoded by a nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, the term “heterologous” indicates that a polypeptide does not originate from a specific cell and the respective encoding nucleic acid has been introduced into said cell by DNA delivery methods, e.g., by transfection, electroporation, or transformation methods. Thus, a heterologous polypeptide is a polypeptide that is artificial to the cell expressing it, whereby this is independent whether the polypeptide is a naturally occurring polypeptide originating from a different cell/organism or is a synthetic polypeptide.

An “isolated” nucleic acid refers to a nucleic acid that has been separated from a component of its natural environment. An isolated nucleic acid includes a nucleic acid contained in cells that ordinarily contain the nucleic acid, but the nucleic acid molecule is present extrachromosomally or at a chromosomal location that is different from its natural chromosomal location.

An “isolated nucleic acid encoding an antibody” refers to one or more nucleic acids encoding the heavy and light chains (or fragments thereof) of the antibody according to the invention. Such nucleic acid(s) include those in a single vector or separate vectors, and such nucleic acid(s) present at one or more locations in a host cell.

The terms “(mammalian) cell” and “(mammalian) cell line” are used interchangeably herein refer to cells into which an exogenous nucleic acid(s) has been introduced, including the progeny of such cells.

A “mammalian cell comprising an exogenous nucleotide sequence” and a “recombinant mammalian cell” are both “transformed cells”. This term includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

The term “nucleic acid” or “polynucleotide” includes any molecule and/or compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, a nucleic acid is described by its sequence of bases, whereby said bases represent the primary structure (linear structure) of a nucleic acid. The sequence of bases is typically represented in 5′- to 3′-direction, i.e. from the 5′-end to the 3′-end. Herein, the term nucleic acid encompasses deoxyribonucleic acid (DNA) including e.g., complementary DNA (cDNA) and genomic DNA as well as synthetic forms of DNA. The nucleic acid may be linear or circular. In addition, the term nucleic acid includes both sense and antisense strands, as well as single stranded and double stranded forms. Moreover, the herein described nucleic acid can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA molecules which are suitable as a vector for direct expression of an antibody according to the invention in vitro and/or in vivo, e.g., in a host or patient. Such DNA (e.g., cDNA) vectors can be unmodified or modified.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, nucleic acid sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located at a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′-end-independent manner.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The term “recombinant mammalian cell” as used herein denotes a mammalian cell comprising an exogenous nucleotide sequence capable of expressing a polypeptide. Such recombinant mammalian cells are cells into which one or more exogenous nucleic acid(s) have been introduced, including the progeny of such cells. Thus, the term “a mammalian cell comprising a nucleic acid encoding an antibody” denotes cells comprising an exogenous nucleic acid integrated in the genome of the mammalian cell and capable of expressing the antibody. In certain embodiments, the mammalian cell comprising an exogenous nucleic acid is a cell comprising an exogenous nucleic acid integrated at a single site within a locus of the genome of the mammalian cell, wherein the exogenous nucleic acid comprises a first and a second recombination recognition sequence flanking at least one first selection marker, and a third recombination recognition sequence located between the first and the second recombination recognition sequence, and all the recombination recognition sequences are different. The integration has been effected in this case by a recombinase mediated cassette exchange (RMCE).

As used herein, the term “selection marker” denotes a nucleic acid that allows cells carrying the nucleic acid to be specifically selected for or against, in the presence of a corresponding selection agent. For example, but not by way of limitation, a selection marker can allow the mammalian cell transformed with the selection marker nucleic acid to be positively selected for in the presence of the respective selection agent (selective cultivation conditions); a non-transformed mammalian cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143. Beyond facilitating a selection in the presence of a corresponding selection agent, a selection marker can alternatively encode a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this nucleic acid, e.g., by the detection or absence, respectively, of the fluorescence emitted by the encoded polypeptide.

The term “signal sequence” or “leader sequence” refers to a sequence of amino acid residues located at the N-terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Non-limiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “treatment,” as used herein, covers any administration or application of a therapeutic for disease in a human, or other mammal, and includes inhibiting the disease or progression of the disease, inhibiting or slowing the disease or its progression, arresting or slowing its development, inhibiting, reducing, or slowing development of at least one symptom of the disease, slowing the time to onset of the disease, preventing onset of at least one disease symptom, slowing the time to onset of at least one disease symptom, partially or fully relieving the disease, or curing the disease, for example, by causing regression, or restoring or repairing a lost, missing, or defective function; or stimulating an inefficient process. The terms “inhibition” or “inhibit” refer to a decrease or cessation of any symptom or phenotypic characteristic or to the decrease or cessation in the incidence, degree, or likelihood of that symptom or characteristic.

The term “vector”, as used herein, refers to a nucleic acid capable of propagating another nucleic acid to which it is linked. The term includes the vector as a self-replicating nucleic acid structure as well as the vector incorporated into the genome of a mammalian cell into which it has been introduced. Certain vectors are capable of directing the expression of nucleic acids to which they are operatively linked. Such vectors are referred to herein as “expression vectors”.

“Affinity” refers to the strength of the sum of all non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). Affinity can be measured by common methods known in the art, including those described herein.

The term “antibody-dependent cellular cytotoxicity (ADCC)” is a function mediated by Fc receptor binding and refers to lysis of target cells by an antibody as reported herein in the presence of effector cells. ADCC can be measured by the treatment of a preparation of CD19 expressing erythroid cells (e.g. K562 cells expressing recombinant human CD19) with an antibody according to the current invention in the presence of effector cells such as freshly isolated PBMC (peripheral blood mononuclear cells) or purified effector cells from buffy coats, like monocytes or NK (natural killer) cells. Target cells are labeled with 51Cr and subsequently incubated with the antibody. The labeled cells are incubated with effector cells and the supernatant is analyzed for released 51Cr. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA).

The term “binding (to an antigen)” denotes the binding of an antibody to its cognate antigen. Binding can be determined in an in vitro assay. In certain embodiments, binding is determined in a binding assay in which the antibody is bound to a surface and binding of the antigen to the antibody is measured by Surface Plasmon Resonance (SPR). The affinity of the binding is defined by the terms ka (rate constant for the association of the antibody from the antibody/antigen complex), kd (dissociation constant), and KD (kd/ka). Thus, binding means a specific and detectable interaction between the antibody and its cognate antigen, e.g. a binding affinity (KD) of 1E−4 M or less. “Specifically binding” means a binding affinity (KD) of 1E−8 M or less, in some embodiments of 1E−13 to 1E−8 M, in some embodiments of 1E−13 to 1E−9 M.

“Effector functions” refer to those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B-cell receptor); and B-cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624);
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells, it seems to function to suppress further immunoglobulin production and isotype switching to, for example, the IgE class. On macrophages, FcγRIIB acts to inhibit phagocytosis as mediated through FcγRIIA. On eosinophils and mast cells, the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat);
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “Fc receptor” as used herein refers to activation receptors characterized by the presence of a cytoplasmic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of the antibody to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434). In certain embodiments, the antibody according to the invention is of IgG1 or IgG2 subclass and comprises the mutation PVA236, GLPSS331, L234A/L235A or P329G/L234A/L235A. In certain embodiments, the antibody as reported herein is of IgG4 subclass and comprises the mutation L235E. In certain embodiments, the antibody according to the invention further comprises the mutation S228P.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated” antibody is one, which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., size exclusion chromatography or ion exchange or reverse phase HPLC) analytical methods. For review of methods for assessment of antibody purity, see, e.g., Flatman, S. et al., J. Chrom. B 848 (2007) 79-87.

An “isolated” nucleic acid refers to a nucleic acid molecule that has been separated from a component of its natural environment.

An “isolated nucleic acid encoding an anti-human Abeta protein antibody” denotes to one or more nucleic acid molecules encoding the antibody heavy and light chains (or fragments thereof), including such nucleic acid molecule(s) in a single plasmid or separate plasmids.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, an antibody according to the current invention is used to delay development of a disease or to slow the progression of a disease.

The term “valent” as used within the current application denotes the presence of a specified number of binding sites in a (antibody) molecule. As such, the terms “bivalent”, “tetravalent”, and “hexavalent” denote the presence of two binding sites, four binding sites, and six binding sites, respectively, in a (antibody) molecule.

The term “variable region” or “variable domain” refers to the domain of an antibody heavy or light chain that is involved in binding of the antibody to its antigen. The variable domains of the heavy chain and light chain (VH and VL, respectively) of an antibody generally have similar structures, with each domain comprising four framework regions (FRs) and three hypervariable regions (HVRs) (see, e.g., Kindt, T. J. et al. Kuby Immunology, 6th ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

### HELM Strings

Molecules used in the current invention may be depicted herein using Hierarchical Editing Language for Macromolecules (HELM) notation.

HELM is a notation format designed to depict the structure of macromolecules. Full details of HELM notation may be found at www.pistoiaalliance.org/helm-tools/, in Zhang et al. J. Chem. Inf. Model. 2012, 52, 2796-2806 (which initially described HELM notation) and in Milton et al. J. Chem. Inf. Model. 2017, 57, 1233-1239 (which describes HELM version 2.0).

Briefly, a macromolecule is depicted as a “HELM string”, which is divided into sections. The first section of the HELM string lists the molecules comprised in the macromolecule. The second section lists the connections between molecules within the macromolecule. Third, fourth and fifth sections (which may be used in HELM strings for more complex macromolecules) are not used in the HELM strings herein. One or more dollar sign $ marks the end of a section and a vertical line | defines sub-sections (e.g. separating molecules in the first section, and separating connections in the second section).

Accordingly, compounds used in the current invention are represented by a HELM string consisting of two sections: the first section defines the structures of antisense strand, the sense strand and (if present) the conjugate moiety, and the second section defines the base-pairing between the strands and how the conjugate moiety (if present) is connected to either strand (typically the sense strand).

Each molecule listed in the first section of a HELM string is given an identifier (e.g. “RNA1” for a nucleic acid, “PEPTIDE1” for an amino acid sequence, “CHEM1” for a chemical structure) and the structure of the molecule is defined by notation in braces { } immediately following the identifier. Thus, in HELM strings depicting compounds used in the current invention, “RNA1” is the identifier of the antisense strand, “RNA2” is the identifier of the sense strand and “CHEM1” is the identifier of the conjugate moiety (if present).

The notation used to define the structure of each molecule in braces { } in the first section of HELM strings for the present invention are as follows:
demarcates nucleosides,
[mR](A) is a 2′-O-methyl RNA adenine nucleoside,
[mR](C) is a 2′-O-methyl RNA cytosine nucleoside,
[mR](G) is a 2′-O-methyl RNA guanine nucleoside,
[mR](U) is a 2′-O-methyl RNA uracil nucleoside,
[fR](A) is a 2′-fluoro RNA adenine nucleoside,
[fR](C) is a 2′-fluoro RNA cytosine nucleoside,
[fR](G) is a 2′-fluoro RNA guanine nucleoside,
[fR](U) is a 2′-fluoro RNA uracil nucleoside,
[P] is a phosphodiester internucleoside linkage, and
[sP] is a phosphorothioate internucleoside linkage.

In HELM strings representing the conjugates used in the current invention there is a connection between the conjugate moiety and sense strand. This connection is represented in all HELM strings herein as follows:
RNA2, CHEM1,57:R2-1: R.

“V2.0” indicates that HELM version 2.0 is used.

### Example of HELM Notation

For Example, siRNA 664 is Represented by the Following HELM String:

siRNA 664
RNA1{[fR](U)[sP].[mR](C)[sP].[fR](U)P.[mR](C)P.[fR](G)P.[mR](U)P.[f
R](G)P.[mR](G)P.[fR](C)P.[mR](C)P.[fR](U)P.[mR](U)P.[fR](A)P.[mR](A)P.[fR
](U)P.[mR](G)P.[fR](A)P.[mR](A)[sP].[fR](A)[sP].[idR](T)}|RNA2{[mR](U)[sP].
[fR](U)[sP].[mR](U)P.[R](C)P.[mR](A)P.[fR](U)P.[mR](U)P.[fR](A)P.[mR](A)P
.[fR](G)P.[mR](G)P.[fR](C)P.[mR](C)P.[fR](A)P.[mR](C)P.[fR](G)P.[mR](A)P.[f
R](G)P.[mR](A)[sP].[mR](U)[sP].[mR](U)}$RNA1,RNA2,11:pair-
47:pair|RNA1,RNA2,14:pair-44:pair|RNA1,RNA2,17:pair-
41:pair|RNA1,RNA2,20:pair-38:pair|RNA1,RNA2,23:pair-
35:pair|RNA1,RNA2,26:pair-32:pair|RNA1,RNA2,29:pair-
29:pair|RNA1,RNA2,2:pair-56:pair|RNA1,RNA2,32:pair-
26:pair|RNA1,RNA2,35:pair-23:pair|RNA1,RNA2,38:pair-
20:pair|RNA1,RNA2,41:pair-17:pair|RNA1,RNA2,44:pair-
14:pair|RNA1,RNA2,47:pair-11:pair|RNA1,RNA2,50:pair-
8:pair|RNA1,RNA2,53:pair-5:pair|RNA1,RNA2,56:pair-
2:pair|RNA1,RNA2,5:pair-53:pair|RNA1,RNA2,8:pair-50:pair$$$V2.0

This HELM string consists of two sections; the end of each section is marked by a $ sign. The first section defines the two components of the compound: the antisense strand (RNA1) and the sense strand (RNA2). The structure of each component follows the name in braces { }. The second section defines how the antisense strand (RNA1) forms base pairs with the sense strand (RNA2). Two further $$ signs mark the end of the HELM string as a whole. “V2.0” indicates that HELM version 2.0 is used.

ASO 297
(SEQ ID NO: 141)
RNA1{[LR](G)[sP].[LR](A)[sP].[LR](G)[sP].[dR](T)[sP].[dR](T)[sP].[dR]
(A)[sP].[dR](C)[sP].[dR](T)[sP].[dR](T)[sP].[dR](G)[sP].[dR](C)[sP].[dR](C)[sP].
[dR](A)[sP].[LR](A)[sP].[LR]([5meC])[sP].[LR](T)}$$$$V2.0
ASO 420
(SEQ ID NO: 139)
RNA1{[LR](T)[sP].[LR](T)[sP].[LR](A)[sP].[LR](A)[sP].[dR](C)[sP].[dR]
(T)[sP].[dR](C)[sP].[dR](A)[sP].[dR](A)[sP].[dR](A)[sP].[dR](T)[sP].[dR](C)[sP].
[dR](A)[sP].[dR](A)[P].[dR](T)[sP].[dR](T)[sP].[LR]([5meC])[sP].[LR](T)[sP].[
LR]([5meC])[sP].[LR](A)}$$$$V2.0
ASO 827
(SEQ ID NO: 140)
RNA1{[LR]([5meC])[sP].[LR](T)[sP].[LR](T)[sP].[LR](T)[sP].[dR](A)[sP
].[dR](A)[sP].[dR](T)[sP].[dR](T)[sP].[dR](T)[sP].[dR](A)[sP].[dR](A)[sP].[dR](T
)[sP].[dR](C)[sP].[dR](A)[sP].[LR]([5meC])[sP].[dR](T)[sP].[LR]([5meC])[sP].[L
R](A)[sP].[LR](T)}$$$$V2.0
ASO 042
(SEQ ID NO: 142)
RNA1{[LR]([5meC])[sP].[dR](C)[P].[LR](T)[sP].[dR](T)[sP].[dR](T)[sP]
.[LR]([5meC])[sP].[dR](A)[sP].[dR](C)[sP].[LR](T)[sP].[dR](C)[sP].[LR](G)[sP].[
dR](T)[sP].[dR](T)[sP].[LR](T)[sP].[dR](C)[sP].[dR](C)[sP].[LR](A)[sP].[LR](G)
}$$$$V2.0
ASO 576
(SEQ ID NO: 135)
CHEM1{[A6]}|CHEM2{[BCN_PEG3]}|RNA1{[sP].[LR](T)[sP].[LR](T)[s
P].[LR](A)[sP].[LR](A)[sP].[dR](C)[sP].[dR](T)[sP].[dR](C)[sP].[dR](A)[sP].[dR]
(A)[sP].[dR](A)[sP].[dR](T)[sP].[dR](C)[sP].[dR](A)[sP].[dR](A)[sP].[dR](T)[sP].
[dR](T)[sP].[LR]([5meC])[sP].[LR](T)[sP].[LR]([5meC])[sP].[LR](A)}$CHEM1,
CHEM2,1:R2-1:R1|CHEM1,RNA1,1:R1-1:R1$$$V2.0
ASO385-sameASOsequenceasSEQIDNO:140ASO827
(SEQ ID NO: 150)
CHEM1{[A6]}|CHEM2{[BCN_PEG3]}|RNA1{[sP].[LR]([5meC])[sP].[LR](T)[sP].[LR](T)
[sP].[LR](T)[sP].[dR](A)[sP].[dR](A)[sP].[dR](T)[sP].[dR](T)[sP].[dR](T)[sP].
[dR](A)[sP].[dR](A)[sP].[dR](T)[sP].[dR](C)[sP].[dR](A)[sP].[LR]([5meC])[sP].
[dR](T)[SP].[LR]([5meC])[sP].[LR](A)[sP].[LR](T)}$CHEM1,CHEM2,1:R2-
1:R1|CHEM1,RNA1,1:R1-1:R1$$$V2.0
siRNA 664
(SEQ ID NO: 128 and 129)
RNA1{[fR](U)[sP].[mR](C)[sP].[fR](U)P.[mR](C)P.[fR](G)P.[mR](U)P.[f
R](G)P.[mR](G)P.[fR](C)P.[mR](C)P.[fR](U)P.[mR](U)P.[fR](A)P.[mR](A)P.[fR
](U)P.[mR](G)P.[fR](A)P.[mR](A)[sP].[fR](A)[sP].[idR](T)}|RNA2{[mR](U)[sP].
[fR](U)[sP].[mR](U)P.[fR](C)P.[mR](A)P.[fR](U)P.[mR](U)P.[fR](A)P.[mR](A)P
.[fR](G)P.[mR](G)P.[fR](C)P.[mR](C)P.[fR](A)P.[mR](C)P.[fR](G)P.[mR](A)P.[f
R](G)P.[mR](A)[sP].[mR](U)[sP].[mR](U)}$RNA1,RNA2,11:pair-
47:pair|RNA1,RNA2,14:pair-44:pair|RNA1,RNA2,17:pair-
41:pair|RNA1,RNA2,20:pair-38:pair|RNA1,RNA2,23:pair-
35:pair|RNA1,RNA2,26:pair-32:pair|RNA1,RNA2,29:pair-
29:pair|RNA1,RNA2,2:pair-56:pair|RNA1,RNA2,32:pair-
26:pair|RNA1,RNA2,35:pair-23:pair|RNA1,RNA2,38:pair-
20:pair|RNA1,RNA2,41:pair-17:pair|RNA1,RNA2,44:pair-
14:pair|RNA1,RNA2,47:pair-11:pair|RNA1,RNA2,50:pair-
8:pair|RNA1,RNA2,53:pair-5:pair|RNA1,RNA2,56:pair-
2:pair|RNA1,RNA2,5:pair-53:pair|RNA1,RNA2,8:pair-50:pair$$$V2.0
ASO 918
(SEQ ID NO: 127)
CHEM1{[BCN_2]}|RNA1{[sP].[LR](T)[sP].[LR](T)[sP].[LR](A)[sP].[LR]
(A)[sP].[dR](C)[sP].[dR](T)[sP].[dR](C)[sP].[dR](A)[sP].[dR](A)[sP].[dR](A)[sP].
[dR](T)[sP].[dR](C)[sP].[dR](A)[sP].[dR](A)[sP].[dR](T)[sP].[dR](T)[sP].[LR]([5
meC])[sP].[LR](T)[sP].[LR]([5meC])[sP].[LR](A)}$CHEM1,RNA1,1:R1-
1:R1$$$V2.0
ASO 307
(SEQ ID NO: 132)
CHEM1{[BCN_2]}|RNA1{[sP].[LR]([5meC])[sP].[LR](T)[sP].[LR](T)[SP
].[LR](T)[sP].[dR](A)[sP].[dR](A)[sP].[dR](T)[sP].[dR](T)[sP].[dR](T)[sP].[dR](
A)[sP].[dR](A)[sP].[dR](T)[sP].[dR](C)[sP].[dR](A)[sP].[LR]([5meC])[sP].[dR](T
)[sP].[LR]([5meC])[sP].[LR](A)[sP].[LR](T)}$CHEM1,RNA1,1:R1-1:R1$$$V2.0
ASO 980
(SEQ ID NO: 133)
RNA1{[dR](T)[sP].[LR]([5meC])[sP].[dR](A)[sP].[dR](C)}$$$$V2.0

### DETAILED DESCRIPTION OF EMBODIMENTS OF THE INVENTION

Herein are reported monoclonal antibodies that specifically bind single-stranded LNAs. In more detail, the antibodies according to the current invention specifically bind to LNAs that harbor specific modifications, but do not bind unmodified single-or double-stranded nucleic acids.

With the antibodies according to the current invention, amongst other things, the cloaking of modules to counteract or ameliorate potential issues of LNAs and LNA-conjugates becomes possible.

With the antibodies according to the current invention, amongst other things, immunoassays can be provided that allow the detection of LNA-specific ADAs and that allow the determination of the pharmacokinetic (PK) properties of ASO-containing compounds.

### Generation of the LNA-Binding Antibodies According to the Current Invention

The antibodies according to the invention have been generated using a deliberate immunization strategy to obtain pan-LNA binding antibodies, i.e. anti-LNA antibodies that bind specifically to the modified nucleotide independent of the overall base sequence of the LNA. Different mouse strains (BALB/c and NMRI mice) were hyperimmunized with selected LNA-moiety containing ASOs (Table 1-1) coupled to keyhole limpet hemocyanine (KLH). Two different immunization schemes were applied, (a) immunization with a mixture of all three immunogens and (b) alternating immunization with individual immunogens.

TABLE 1-1
Used immunogens.

| | | SEQ |
|---|---|---|
| | nucleotide | ID |
| immunogen | sequence | NO: |
| LNA 1 - Gapmer1-3′ | 5′-GTCtctt | 01 |
| | taccTGG-3′ | |
| LNA 2 - Gapmer2-3′ | 5′-ACTccaa | 02 |
| | atcCTG-3′ | |
| LNA 4b - Gapmer4b-3′ | 5′-AAGtctg | 03 |
| | tttcACC-3′ | |

The immune responses in individual animals were checked by immunoassay titer analysis of sera using biotinylated versions of the three LNA immunogens and corresponding DNA controls. Animals with highest titer were used for the isolation of B-cells for the generation of hybridomas.

TABLE 1-2
Titer analysis of hyperimmunized animals (animals used for hybridoma generation are marked by an asterix (*)).

| | mouse | immunization | Test item - Titer [Dilution] | | | | | | |
|---|---|---|---|---|---|---|---|---|---|
| animal ID | strain | scheme | LNA1-Bi | DNA1-Bi | LNA2-Bi | DNA2-Bi | LNA4b-Bi | DNA4b-Bi | blank |
| K2312M1 | Balb/c | LNA 1, 2 | 4900 | 2100 | 5300 | 2200 | 3300 | 2200 | 2000 |
| K2312M2 | Balb/c | and 4b as | 7200 | 2600 | 9700 | 600 | 8300 | 600 | 0 |
| K2312M3 | Balb/c | mixture | 6100 | 1300 | 3200 | 500 | 5400 | 500 | 0 |
| K2312M4 | Balb/c | | 7700 | 1200 | 6700 | 700 | 100 | 700 | 0 |
| K2312M5 | Balb/c | | 7300 | 1300 | 6500 | 1400 | 100 | 1400 | 0 |
| K2313M1 | NMRI | | 5800 | 2900 | 4900 | 100 | 6800 | 100 | 3500 |
| K2313M2 | NMRI | | 4700 | 1100 | 5100 | 1700 | 2900 | 1700 | 0 |
| K2313M3* | NMRI | | 10400 | 2000 | 8800 | 2600 | 9400 | 2600 | 800 |
| K2313M4 | NMRI | | 9000 | 4700 | 6000 | 4600 | 7000 | 4600 | 5600 |
| K2313M5 | NMRI | | 2900 | 1200 | 2400 | 800 | 2400 | 800 | 0 |
| K2314M1 | Balb/c | | 7900 | 2400 | 3600 | 700 | 6100 | 1600 | 0 |
| K2314M2 | Balb/c | LNA 1, 2 | 3200 | 1100 | 2000 | 500 | 2500 | 700 | 200 |
| K2314M3 | Balb/c | and 4b | 9600 | 2800 | 8200 | 1000 | 9000 | 2400 | 0 |
| K2314M4 | Balb/c | alternating | 10500 | 5100 | 8000 | 900 | 8600 | 2000 | 300 |
| K2314M5 | Balb/c | | 6300 | 2300 | 2100 | 600 | 2500 | 600 | 0 |
| K2315M1 | NMRI | | 8400 | 2200 | 4100 | 600 | 6100 | 1800 | 0 |
| K2315M2 | NMRI | | 10000 | 8300 | 11200 | 7600 | 9800 | 6700 | 8000 |
| K2315M3* | NMRI | | 17200 | 5100 | 18500 | 2500 | 16600 | 3000 | 400 |
| K2315M4 | NMRI | | 11500 | 6200 | 10200 | 3600 | 9100 | 3200 | 0 |
| K2315M5 | NMRI | | 12200 | 8700 | 11500 | 8500 | 11100 | 9000 | 7300 |

From two selected animals, spleen cells were fused to Ag8 cells to generate antibody-producing hybridomas using state-of-the-art hybridoma cell technology. After cell fusion, hybridomas were screened for specific reactivity with LNA-containing ASOs using DNA-containing ASOs for specificity evaluation.

In more detail, from two cell fusions, 3840 initial hybridoma clones resulting in 33 primary hybridoma clones were obtained. Thereof, 11 were selected for further characterization of binding characterization by biomolecular interaction analysis (kinetic and thermodynamic surface plasmon resonance). Five primary hybridoma clones were further processed by subcloning to generate monoclonal hybridoma cell culture clones. The respective data is shown in Tables 1-3 and 1-4.

TABLE 1-3
Results of analysis of hybridoma antibody specificity.

| | signal [OD] | | | |
|---|---|---|---|---|
| hybridoma/ | | | | mixture of |
| clone | LNA1-Bi | LNA2-Bi | LNA4b-Bi | DNA1-2-4b-Bi |
| 1.0.1 | 0.190 | 0.834 | 1.877 | 0.053 |
| 1.0.2 | 0.063 | 0.488 | 1.606 | 0.029 |
| 1.0.3 | 0.093 | 0.267 | 1.827 | 0.029 |
| 1.0.4 | 0.153 | 1.199 | 1.343 | 0.032 |
| 1.0.5 | 0.193 | 1.007 | 2.072 | 0.046 |
| 1.0.6 | 0.270 | 1.021 | 2.160 | 0.042 |
| 1.0.7 | 0.047 | 0.159 | 1.437 | 0.037 |
| 1.0.8 | 1.401 | 0.811 | 0.500 | 0.038 |
| 1.0.9 | 0.110 | 0.812 | 1.937 | 0.033 |
| 1.0.10 | 1.281 | 1.339 | 1.134 | 0.046 |
| 1.0.11 | 0.286 | 1.145 | 1.867 | 0.084 |
| 1.0.12 | 0.048 | 0.177 | 1.403 | 0.030 |
| 1.0.13 | 1.232 | 1.089 | 0.694 | 0.043 |
| 1.0.14 | 0.193 | 0.846 | 1.806 | 0.050 |
| 1.0.15 | 0.541 | 1.139 | 2.066 | 0.097 |
| 1.0.16 | 0.101 | 0.343 | 1.828 | 0.042 |
| 1.0.17 | 0.103 | 0.757 | 1.902 | 0.036 |
| 1.0.18 | 0.098 | 0.798 | 1.919 | 0.040 |
| 1.0.19 | 0.045 | 0.336 | 1.661 | 0.033 |
| 1.0.20 | 0.289 | 1.246 | 2.203 | 0.049 |
| 1.0.21 | 1.865 | 1.901 | 1.124 | 0.030 |
| 1.0.22 | 0.047 | 0.163 | 1.121 | 0.031 |
| 1.0.23 | 0.063 | 0.202 | 1.511 | 0.035 |
| 1.0.24 | 0.046 | 0.239 | 1.086 | 0.034 |
| 1.0.25 | 0.121 | 0.797 | 1.992 | 0.036 |
| 1.0.26 | 0.052 | 0.203 | 1.463 | 0.030 |
| 1.0.27 | 0.037 | 0.172 | 1.309 | 0.028 |
| 1.0.28 | 0.083 | 0.464 | 1.637 | 0.029 |
| 1.0.29 | 0.122 | 0.668 | 1.850 | 0.049 |
| 1.0.30 | 0.650 | 0.601 | 0.381 | 0.031 |
| 1.0.31 | 0.048 | 0.230 | 1.341 | 0.030 |
| 1.0.32 | 0.035 | 0.043 | 0.413 | 0.031 |
| 2.0.33 | 0.190 | 0.055 | 1.154 | 0.035 |

TABLE 1-4
Characterization and specificity evaluation of binding
properties of hybridoma antibody subclones.

| | | | Test item - Signal [OD] | | | | | |
|---|---|---|---|---|---|---|---|---|
| | Ig | | LNA1- | DNA1- | LNA2- | DNA2- | LNA4b- | DNA4b- |
| clone | subclass | LC | Bi | Bi | Bi | Bi | Bi | Bi |
| 1.2.8 | IgG1 | kappa | 2.330 | 0.041 | 2.012 | 0.037 | 1.916 | 0.034 |
| 1.4.10 | IgG2b | kappa | 3.398 | 0.084 | 3.154 | 0.049 | 2.977 | 0.038 |
| 1.5.13 | IgG2b | kappa | 2.717 | 0.078 | 2.729 | 0.036 | 2.574 | 0.039 |
| 1.7.15 | IgG2b | kappa | 2.195 | 0.060 | 2.815 | 0.150 | >4.000 | 0.035 |
| 1.9.21 | IgG2b | kappa | >4.000 | 0.064 | >4.000 | 0.039 | 3.556 | 0.110 |

### Anti-LNA Antibodies According to the Current Invention

For determining the amino acid sequences of the variable heavy chain (VH) and light chain (LC) domain of the five selected antibodies the corresponding cDNAs were sequenced. Shortly, mRNA was extracted from the hybridoma cell pellets, followed by cDNA generation by reverse-transcription with an oligo(dT) primer. Amplification of VH and VL regions was performed by PCR using variable domain primers. Finally, The VH and VL products were cloned into a sequencing vector, transformed into competent E. coli cells and screened by PCR for positive transformants. Selected colonies were picked and analyzed by DNA sequencing.

The derived protein consensus sequences from multiple cDNA readings are summarized in the following Table 2-1. The signal peptide sequence (if present) is shown by normal letters at the N-terminal end of the sequence. The variable domain is shown by underlining. The hypervariable regions (HVRs) are shown by underlining and in bold letters (determined according to Kabat).

TABLE 2-1
Consensus annotated amino acid sequence of the variable domains
of anti-LNA antibodies according to the current invention.

| antibody | | | SEQ |
|---|---|---|---|
| produced | | consensus amino acid sequence | ID |
| by clone | domain | (N- to C-terminal direction) | NO: |
| 1.7.15 | VH | MAVLGLLFCLVTFPSCVLS | 04 |
| | | QVQLKQSGPSFVQPSQSLSITCTVSGFSLT | 05 |
| | | TYGVH | 06 |
| | | WVRQSPGKGLEWLG | 07 |
| | | VIWRSGGTDYNAAFMS | 08 |
| | | RLSITKDNSKSQVFFKMNSLQTDDTAIYYCTN | 09 |
| | | LYY | 10 |
| | | WGQGTTLTVSS | 11 |
| | | (complete VH without signal peptide: SEQ ID NO: 12) | |
| | VL | MSVPTQVLGLLVLWIPGSTA | 13 |
| | (kappa) | DIVMTQATFSNPVTLGTSVSISC | 14 |
| | | RSSKSLLRSNGITYLY | 15 |
| | | WYLQKPGQSPQLLIY | 16 |
| | | QMSNLAS | 17 |
| | | GVPDRESSSGSGTDFTLRISRVEAEDVGVYYC | 18 |
| | | GQNLELPYT | 19 |
| | | FGSGTKVEIK | 20 |
| | | (complete VL without signal peptide: SEQ ID NO: 21) | |
| 1.2.8 | VH | MAVLGLFFCLVAFPSCVLS | 22 |
| | | QIQLRESGPGLVAPSQSLSITCTVSGFSLT | 23 |
| | | NYGVH | 24 |
| | | WVRQPPGKGLEWLG | 25 |
| | | VIWAGGSTIYNSALMS | 26 |
| | | RLSISRDNSKSQVFLEMNSLQTYDTAMYYCA | 27 |
| | | RDRPFDGFFFSY | 28 |
| | | WGQGTALTVSS | 29 |
| | | (complete VH without signal peptide: SEQ ID NO: 30) | |
| | VL | MKLPVRLLVLMFWIPACNS | 31 |
| | (kappa) | DVLMTQTPLSLPVSLGDQASISC | 32 |
| | | RSSQAIVHRSGNTFLE | 33 |
| | | WYLQKPGQSPKLLIY | 34 |
| | | KVSNRFS | 35 |
| | | GVPDRFSGSGSGTDFTLKINRVEAEDLGVYYC | 36 |
| | | FQGSHVPYT | 37 |
| | | FGSGTKLEIK | 38 |
| | | (complete VL without signal peptide: SEQ ID NO: 39) | |
| 1.9.21 | VH | MEWSWVFLFLLSVTAGVHS | 40 |
| | | QVQLQQSGAELVRPGTSVKVSCKASGYAFT | 41 |
| | | NYLIE | 42 |
| | | WIKQRPGQGLEWIG | 43 |
| | | VINPGSGGTNYNEKFRV | 44 |
| | | KATLTADKSSSTAYMQLSSLTSDDSAVYFCAR | 45 |
| | | GGGYY | 46 |
| | | WGQGTLVTVSA | 47 |
| | | (complete VH without signal peptide: SEQ ID NO: 48) | |
| | VL | MVSSAQFLGLLVLWIPGAIG | 49 |
| | (kappa) | DIVMTQAAPSVPVTPGESVSISC | 50 |
| | | RSSKSLLHSNGNTYLF | 51 |
| | | WFLQRPGQSPQVLIY | 52 |
| | | RMSNLAS | 53 |
| | | GVPDRFSGSGSGTAFTLRISRVEAEDVGVYYC | 54 |
| | | MQHLEYPYT | 55 |
| | | FGSGTRLEIK | 56 |
| | | (complete VL without signal peptide: SEQ ID NO: 57) | |
| 1.4.10 | VH | QIQLRESGPGLVAPSQSLSITCTVSGFSLV | 58 |
| | | NYGIH | 59 |
| | | WVRQPPGKGLEWLG | 60 |
| | | VIWAGGSTIYNSALMS | 61 |
| | | RLSISRDNSKNQVFLEMNSLQTYDTAMYYCAR | 62 |
| | | DRPFDGFFFSY | 63 |
| | | WGQGTALTVSS | 64 |
| | | (complete VH without signal peptide: SEQ ID NO: 65) | |
| | VL | DVLMTQTPLSLPVSLGDQASISC | 66 |
| | (kappa) | RSSQAIVHRSGNTFLE | 67 |
| | | WYLQKPGQSPKLLIY | 68 |
| | | KVSNRFS | 69 |
| | | GVPDRFSGSGSGTDFTLKINRVEAEDLGVYYC | 70 |
| | | FQGSHVPYT | 71 |
| | | FGSGTKLEIK | 72 |
| | | (complete VL without signal peptide: SEQ ID NO: 73) | |
| 1.5.13 | VH | QVQLKESGPGLVAPSQSLSITCTISGFSLT | 74 |
| | | TYGVH | 75 |
| | | WVRQPPGMGLEWLV | 76 |
| | | VIWGDGSTTYNSALRS | 77 |
| | | RLSISKDNSRSQVFLKMNSLQTDDTAMYYCAR | 78 |
| | | HGGRYALDY | 79 |
| | | WGQGTSVTVSS | 80 |
| | | (complete VH without signal peptide: SEQ ID NO: 81) | |
| | VL | DIVMTQSPATLSVTPGDRVSLSC | 82 |
| | | RASQSIGGYLH | 83 |
| | | WYQQKSHESPRLLIK | 84 |
| | | YASQSMS | 85 |
| | | GIPPRFSGSGSGSDFTLSINSVAPEDVGVYYC | 86 |
| | | QAGHTFPLT | 87 |
| | | FGAGTKL | 88 |
| | | (complete VL without signal peptide: SEQ ID NO: 89) | |

Thus, the invention comprises at least the following embodiments:

In one aspect, the invention provides an anti-LNA antibody comprising three VH HVR sequences selected from the group consisting of
(1) (a) HVR-H1 comprising the amino acid sequence of SEQ ID NO: 06;
   (b) HVR-H2 comprising the amino acid sequence of SEQ ID NO: 08; and
   (c) HVR-H3 comprising the amino acid sequence of SEQ ID NO: 10;
(2) (a) HVR-H1 comprising the amino acid sequence of SEQ ID NO: 24;
   (b) HVR-H2 comprising the amino acid sequence of SEQ ID NO: 26; and
   (c) HVR-H3 comprising the amino acid sequence of SEQ ID NO: 28;
(3) (a) HVR-H1 comprising the amino acid sequence of SEQ ID NO: 42;
   (b) HVR-H2 comprising the amino acid sequence of SEQ ID NO: 44; and
   (c) HVR-H3 comprising the amino acid sequence of SEQ ID NO: 46;
(4) (a) HVR-H1 comprising the amino acid sequence of SEQ ID NO: 59;
   (b) HVR-H2 comprising the amino acid sequence of SEQ ID NO: 61; and
   (c) HVR-H3 comprising the amino acid sequence of SEQ ID NO: 63;
(5) (a) HVR-H1 comprising the amino acid sequence of SEQ ID NO: 75;
   (b) HVR-H2 comprising the amino acid sequence of SEQ ID NO: 77; and
   (c) HVR-H3 comprising the amino acid sequence of SEQ ID NO: 79.

In one aspect, the invention provides an anti-LNA antibody comprising three VL HVR sequences selected from the group consisting of
(1) (d) HVR-L1 comprising the amino acid sequence of SEQ ID NO: 15;
   (e) HVR-L2 comprising the amino acid sequence of SEQ ID NO: 17; and
   (f) HVR-L3 comprising the amino acid sequence of SEQ ID NO: 19;
(2) (d) HVR-L1 comprising the amino acid sequence of SEQ ID NO: 33;
   (e) HVR-L2 comprising the amino acid sequence of SEQ ID NO: 35; and
   (f) HVR-L3 comprising the amino acid sequence of SEQ ID NO: 37;
(3) (d) HVR-L1 comprising the amino acid sequence of SEQ ID NO: 51;
   (e) HVR-L2 comprising the amino acid sequence of SEQ ID NO: 53; and
   (f) HVR-L3 comprising the amino acid sequence of SEQ ID NO: 55;
(4) (d) HVR-L1 comprising the amino acid sequence of SEQ ID NO: 67;
   (e) HVR-L2 comprising the amino acid sequence of SEQ ID NO: 69; and
   (f) HVR-L3 comprising the amino acid sequence of SEQ ID NO: 71;
(5) (d) HVR-L1 comprising the amino acid sequence of SEQ ID NO: 83;
   (e) HVR-L2 comprising the amino acid sequence of SEQ ID NO: 85; and
   (f) HVR-L3 comprising the amino acid sequence of SEQ ID NO: 87.

In one aspect, the invention provides an anti-LNA antibody comprising six HVRs selected from the group consisting of
(1) (a) HVR-H1 comprising the amino acid sequence of SEQ ID NO: 06;
   (b) HVR-H2 comprising the amino acid sequence of SEQ ID NO: 08;
   (c) HVR-H3 comprising the amino acid sequence of SEQ ID NO: 10;
   (d) HVR-L1 comprising the amino acid sequence of SEQ ID NO: 15;
   (e) HVR-L2 comprising the amino acid sequence of SEQ ID NO: 17; and
   (f) HVR-L3 comprising the amino acid sequence of SEQ ID NO: 19;
(2) (a) HVR-H1 comprising the amino acid sequence of SEQ ID NO: 24;
   (b) HVR-H2 comprising the amino acid sequence of SEQ ID NO: 26;
   (c) HVR-H3 comprising the amino acid sequence of SEQ ID NO: 28;
   (d) HVR-L1 comprising the amino acid sequence of SEQ ID NO: 33;
   (e) HVR-L2 comprising the amino acid sequence of SEQ ID NO: 35; and
   (f) HVR-L3 comprising the amino acid sequence of SEQ ID NO: 37;
(3) (a) HVR-H1 comprising the amino acid sequence of SEQ ID NO: 42;
   (b) HVR-H2 comprising the amino acid sequence of SEQ ID NO: 44;
   (c) HVR-H3 comprising the amino acid sequence of SEQ ID NO: 46;
   (d) HVR-L1 comprising the amino acid sequence of SEQ ID NO: 51;
   (e) HVR-L2 comprising the amino acid sequence of SEQ ID NO: 53; and
   (f) HVR-L3 comprising the amino acid sequence of SEQ ID NO: 55;
(4) (a) HVR-H1 comprising the amino acid sequence of SEQ ID NO: 59;
   (b) HVR-H2 comprising the amino acid sequence of SEQ ID NO: 61;
   (c) HVR-H3 comprising the amino acid sequence of SEQ ID NO: 63;
   (d) HVR-L1 comprising the amino acid sequence of SEQ ID NO: 67;
   (e) HVR-L2 comprising the amino acid sequence of SEQ ID NO: 69; and
   (f) HVR-L3 comprising the amino acid sequence of SEQ ID NO: 71;
(5) (a) HVR-H1 comprising the amino acid sequence of SEQ ID NO: 75;
   (b) HVR-H2 comprising the amino acid sequence of SEQ ID NO: 77;
   (c) HVR-H3 comprising the amino acid sequence of SEQ ID NO: 79;
   (d) HVR-L1 comprising the amino acid sequence of SEQ ID NO: 83;
   (e) HVR-L2 comprising the amino acid sequence of SEQ ID NO: 85; and
   (f) HVR-L3 comprising the amino acid sequence of SEQ ID NO: 87.

In another aspect, an anti-LNA antibody of the invention comprises
(a) a VH domain comprising three VH HVR sequences selected from the group consisting of
(1) (a) HVR-H1 comprising the amino acid sequence of SEQ ID NO: 06;
   (b) HVR-H2 comprising the amino acid sequence of SEQ ID NO: 08; and
   (c) HVR-H3 comprising the amino acid sequence of SEQ ID NO: 10;
(2) (a) HVR-H1 comprising the amino acid sequence of SEQ ID NO: 24;
   (b) HVR-H2 comprising the amino acid sequence of SEQ ID NO: 26; and
   (c) HVR-H3 comprising the amino acid sequence of SEQ ID NO: 28;
(3) (a) HVR-H1 comprising the amino acid sequence of SEQ ID NO: 42;
   (b) HVR-H2 comprising the amino acid sequence of SEQ ID NO: 44; and
   (c) HVR-H3 comprising the amino acid sequence of SEQ ID NO: 46;
(4) (a) HVR-H1 comprising the amino acid sequence of SEQ ID NO: 59;
   (b) HVR-H2 comprising the amino acid sequence of SEQ ID NO: 61; and
   (c) HVR-H3 comprising the amino acid sequence of SEQ ID NO: 63;
(5) (a) HVR-H1 comprising the amino acid sequence of SEQ ID NO: 75;
   (b) HVR-H2 comprising the amino acid sequence of SEQ ID NO: 77; and
   (c) HVR-H3 comprising the amino acid sequence of SEQ ID NO: 79; and
(b) a VL domain comprising three VL HVR sequences selected from the group consisting of
(1) (d) HVR-L1 comprising the amino acid sequence of SEQ ID NO: 15;
   (e) HVR-L2 comprising the amino acid sequence of SEQ ID NO: 17; and
   (f) HVR-L3 comprising the amino acid sequence of SEQ ID NO: 19;
(2) (d) HVR-L1 comprising the amino acid sequence of SEQ ID NO: 33;
   (e) HVR-L2 comprising the amino acid sequence of SEQ ID NO: 35; and
   (f) HVR-L3 comprising the amino acid sequence of SEQ ID NO: 37;
(3) (d) HVR-L1 comprising the amino acid sequence of SEQ ID NO: 51;
   (e) HVR-L2 comprising the amino acid sequence of SEQ ID NO: 53; and
   (f) HVR-L3 comprising the amino acid sequence of SEQ ID NO: 55;
(4) (d) HVR-L1 comprising the amino acid sequence of SEQ ID NO: 67;
   (e) HVR-L2 comprising the amino acid sequence of SEQ ID NO: 69; and
   (f) HVR-L3 comprising the amino acid sequence of SEQ ID NO: 71;
(5) (d) HVR-L1 comprising the amino acid sequence of SEQ ID NO: 83;
   (e) HVR-L2 comprising the amino acid sequence of SEQ ID NO: 85; and
   (f) HVR-L3 comprising the amino acid sequence of SEQ ID NO: 87.

In any of the aspects provided herein, an anti-LNA antibody according to the invention is a humanized antibody. In one aspect, an anti-LNA antibody according to the invention further comprises besides the HVRs as outlined above an acceptor human framework, e.g. a human immunoglobulin framework or a human consensus framework. Thus, in another aspect, an anti-LNA antibody according to the invention comprises a VH domain comprising a HC-FR1, a HC-FR2, a HC-FR3 and a HC-FR4 each independently of each other of at least 85%, 86%, 87%, 88%, 89%, 90%, 91%, 92%, 93%, 94%, 95%, 96%, 97%, 98% or 99% sequence identity to a respective human germline FR sequence; and a VL domain comprising a LC-FR1, a LC-FR2, a LC-FR3 and a LC-FR4 each independently of each other of at least 85%, 86%, 87%, 88%, 89%, 90%, 91%, 92%, 93%, 94%, 95%, 96%, 97%, 98% or 99% sequence identity to a respective human germline FR sequence.

In another aspect, an anti-LNA antibody according to the invention comprises the three HVR sequences of the VH of
(1) SEQ ID NO: 12;
(2) SEQ ID NO: 30;
(3) SEQ ID NO: 48;
(4) SEQ ID NO: 65; or
(5) SEQ ID NO: 81.

In another aspect, an anti-LNA antibody according to the invention comprises the three HVR sequences of the VL of
(1) SEQ ID NO: 21;
(2) SEQ ID NO: 39;
(3) SEQ ID NO: 57;
(4) SEQ ID NO: 73; or
(5) SEQ ID NO: 89.

In another aspect, an anti-LNA antibody according to the invention comprises the six HVR sequences of the VH and VL of
(1) SEQ ID NO: 12 and 21;
(2) SEQ ID NO: 30 and 39;
(3) SEQ ID NO: 48 and 57;
(4) SEQ ID NO: 65 and 73; or
(5) SEQ ID NO: 81 and 89.

In a further aspect, an anti-LNA antibody comprises the HVR-H1, HVR-H2 and HVR-H3 amino acid sequences of the VH domain of
(1) SEQ ID NO: 12;
(2) SEQ ID NO: 30;
(3) SEQ ID NO: 48;
(4) SEQ ID NO: 65; or
(5) SEQ ID NO: 81;
and the HVR-L1, HVR-L2 and HVR-L3 amino acid sequences of the VL domain of
(1) SEQ ID NO: 21;
(2) SEQ ID NO: 39;
(3) SEQ ID NO: 57;
(4) SEQ ID NO: 73; or
(5) SEQ ID NO: 89.

In another aspect, an anti-LNA antibody comprises a heavy chain variable domain (VH) sequence having at least 90%, 91%, 92%, 93%, 94%, 95%, 96%, 97%, 98%, 99%, or 100% sequence identity to the amino acid sequence of
(1) SEQ ID NO: 12;
(2) SEQ ID NO: 30;
(3) SEQ ID NO: 48;
(4) SEQ ID NO: 65; or
(5) SEQ ID NO: 81.

In one aspect, an anti-LNA antibody comprises a heavy chain variable domain (VH) sequence having at least 95%, sequence identity to the amino acid sequence of
(1) SEQ ID NO: 12;
(2) SEQ ID NO: 30;
(3) SEQ ID NO: 48;
(4) SEQ ID NO: 65; or
(5) SEQ ID NO: 81.

In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-LNA antibody comprising that sequence retains the ability to bind to LNA. In certain aspects, a total of 1 to 10 amino acids have been substituted, inserted and/or deleted in
(1) SEQ ID NO: 12;
(2) SEQ ID NO: 30;
(3) SEQ ID NO: 48;
(4) SEQ ID NO: 65; or
(5) SEQ ID NO: 81.

In certain aspects, substitutions, insertions, or deletions occur in regions outside the HVRs (i.e., in the FRs). Optionally, the anti-LNA antibody comprises the VH sequence of
(1) SEQ ID NO: 12;
(2) SEQ ID NO: 30;
(3) SEQ ID NO: 48;
(4) SEQ ID NO: 65; or
(5) SEQ ID NO: 81;
optionally including post-translational modifications of that sequence.

In another aspect, an anti-LNA antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of
(1) SEQ ID NO: 21;
(2) SEQ ID NO: 39;
(3) SEQ ID NO: 57;
(4) SEQ ID NO: 73; or
(5) SEQ ID NO: 89.

In one aspect, an anti-LNA antibody comprises a light chain variable domain (VL) sequence having at least 95% sequence identity to the amino acid sequence of (1) SEQ ID NO: 21;
(2) SEQ ID NO: 39;
(3) SEQ ID NO: 57;
(4) SEQ ID NO: 73; or
(5) SEQ ID NO: 89.

In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-LNA antibody comprising that sequence retains the ability to bind to LNA. In certain aspects, a total of 1 to 10 amino acids have been substituted, inserted and/or deleted in
(1) SEQ ID NO: 21;
(2) SEQ ID NO: 39;
(3) SEQ ID NO: 57;
(4) SEQ ID NO: 73; or
(5) SEQ ID NO: 89.

In certain aspects, the substitutions, insertions, or deletions occur in regions outside the HVRs (i.e., in the FRs). Optionally, the anti-LNA antibody comprises the VL sequence of
(1) SEQ ID NO: 21;
(2) SEQ ID NO: 39;
(3) SEQ ID NO: 57;
(4) SEQ ID NO: 73; or
(5) SEQ ID NO: 89;
optionally including post-translational modifications of that sequence.

In another aspect, an anti-LNA antibody is provided, wherein the antibody comprises a VH sequence as in any of the aspects provided above, and a VL sequence as in any of the aspects provided above. In one aspect, the antibody comprises the VH and VL sequences of
(1) SEQ ID NO: 12 and 21;
(2) SEQ ID NO: 30 and 39;
(3) SEQ ID NO: 48 and 57;
(4) SEQ ID NO: 65 and 73; or
(5) SEQ ID NO: 81 and 89, respectively;
optionally including post-translational modifications of those sequences.

In a further aspect of the invention, an anti-LNA antibody according to any of the above aspects is a monoclonal antibody, including a chimeric, humanized or human antibody. In one aspect, an anti-LNA antibody is an antibody fragment, e.g., an Fv, Fab, Fab′, scFv, diabody, or F(ab′)2 fragment.

In another aspect, the antibody is a full length antibody, e.g., an intact IgG1 antibody or other antibody class or isotype as defined herein.

In a further aspect, the antibodies according to the invention are of IgG1 isotype/subclass and comprise a constant heavy chain region with the amino acid sequence of SEQ ID NO: 90 or one or more domains of the constant heavy chain region with the amino acid sequence of SEQ ID NO: 90.

In a further aspect, the antibodies according to the current invention comprise a VH and VL sequence as in any of the above embodiments and
a) a first and a second Fc-region polypeptide each of the human subclass IgG1, preferably of SEQ ID NO: 90, or
b) a first and a second Fc-region polypeptide each of the human subclass IgG4, preferably of SEQ ID NO: 91, or
c) a first and a second Fc-region polypeptide each of the human subclass IgG1 each with the mutations L234A, L235A and P329G, preferably of SEQ ID NO: 92 (numbering according to Kabat EU index),
d) a first and a second Fc-region polypeptide each of the human subclass IgG4 with the mutations S228P and L235E, preferably of SEQ ID NO: 93 (numbering according to Kabat EU index),
e) a first and a second Fc-region polypeptide each of the human subclass IgG1 with the mutations L234A, L235A and P329G and the mutation T366W in one Fc-region polypeptide and the mutations T366S, L368A and Y407V in the respective other Fc-region polypeptide, preferably of SEQ ID NO: 94 and 95 (numbering according to Kabat EU index),
f) a first and a second Fc-region polypeptide each of the human subclass IgG1 with the mutations L234A, L235A and P329G and the mutations i) T366W, and ii) S354C or Y349C, in one Fc-region polypeptide and the mutations i) T366S, L368A, and Y407V, and ii) Y349C or S354C, in the respective other Fc-region polypeptide, preferably of SEQ ID NO: 96 and 97 or SEQ ID NO: 98 and 99 (numbering according to Kabat EU index),
g) a first and a second Fc-region polypeptide each of the human subclass IgG4 with the mutations S228P and L235E and the mutation T366W in one Fc-region polypeptide and the mutations T366S, L368A and Y407V in the respective other Fc-region polypeptide, preferably of SEQ ID NO: 100 and 101 (numbering according to Kabat EU index),
h) a first and a second Fc-region polypeptide each of the human subclass IgG4 with the mutations S228P and L235E and the mutations i) T366W, and ii) S354C or Y349C, in one Fc-region polypeptide and the mutations i) T366S, L368A, and Y407V, and ii) Y349C or S354C, in the respective other Fc-region polypeptide, preferably of SEQ ID NO: 102 and 103 or SEQ ID NO: 104 and 105 (numbering according to Kabat EU index),
i) a first and a second Fc-region polypeptide each of the human subclass IgG1 with the mutations L234A, L235A, P329G, I253A, H310A and H435A and the mutation T366W in one Fc-region polypeptide and the mutations T366S, L368A and Y407V in the respective other Fc-region polypeptide, preferably of SEQ ID NO: 106 and 107 (numbering according to Kabat EU index),
j) a first and a second Fc-region polypeptide each of the human subclass IgG1 with the mutations L234A, L235A, P329G, I253A, H310A and H435A and the mutations i) T366W, and ii) S354C or Y349C, in one Fc-region polypeptide and the mutations i) T366S, L368A, and Y407V, and ii) Y349C or S354C, in the respective other Fc-region polypeptide, preferably of SEQ ID NO: 108 and 109 or SEQ ID NO: 110 and 111 (numbering according to Kabat EU index)
k) a first and a second Fc-region polypeptide each of the human subclass IgG1 with the mutations L234A, L235A, P329G, M252Y, S254T and T256E and the mutation T366W in one Fc-region polypeptide and the mutations T366S, L368A and Y407V in the respective other Fc-region polypeptide, preferably of SEQ ID NO: 112 and 113 (numbering according to Kabat EU index)
l) a first and a second Fc-region polypeptide each of the human subclass IgG1 with the mutations L234A, L235A, P329G, M252Y, S254T and T256E and the mutations i) T366W, and ii) S354C or Y349C, in one Fc-region polypeptide and the mutations i) T366S, L368A, and Y407V, and ii) Y349C or S354C, in the respective other Fc-region polypeptide, preferably of SEQ ID NO: 114 and 115 or SEQ ID NO: 116 and 117 (numbering according to Kabat EU index)
m) a first and a second Fc-region polypeptide each of the human subclass IgG1 with the mutations L234A, L235A, P329G, H310A, H433A and Y436A and the mutation T366W in one Fc-region polypeptide and the mutations T366S, L368A and Y407V in the respective other Fc-region polypeptide, preferably of SEQ ID NO: 118 and 119 (numbering according to Kabat EU index), or
n) a first and a second Fc-region polypeptide each of the human subclass IgG1 with the mutations L234A, L235A, P329G, H310A, H433A and Y436A and the mutations i) T366W, and ii) S354C or Y349C, in one Fc-region polypeptide and the mutations i) T366S, L368A, and Y407V, and ii) Y349C or S354C, in the respective other Fc-region polypeptide, preferably of SEQ ID NO: 120 and 121 or SEQ ID NO: 122 and 123 (numbering according to Kabat EU index).

In a further aspect, the antibodies according to the current invention comprise a VH and VL sequence as in any of the above embodiments and
a) a full length constant region of the human subclass IgG1, or
b) a full length constant region of the human subclass IgG4, or
c) a full length constant region of the human subclass IgG1 with the mutations L234A, L235A and P329G (numbering according to Kabat EU index),
d) a full length constant region of the human subclass IgG4 with the mutations S228P and L235E (numbering according to Kabat EU index),
e) a full length constant region of the human subclass IgG1 with the mutations L234A, L235A and P329G in both heavy chains and the mutation T366W in one heavy chain and the mutations T366S, L368A and Y407V in the respective other heavy chain (numbering according to Kabat EU index),
f) a full length constant region of the human subclass IgG1 with the mutations L234A, L235A and P329G in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
g) a full length constant region of the human subclass IgG4 with the mutations S228P and L235E in both heavy chains and the mutation T366W in one heavy chain and the mutations T366S, L368A and Y407V in the respective other heavy chain (numbering according to Kabat EU index),
h) a full length constant region of the human subclass IgG4 with the mutations S228P and L235E in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index),
i) a full length constant region of the human subclass IgG1 with the mutations L234A, L235A, P329G, I253A, H310A and H435A in both heavy chains and the mutation T366W in one heavy chain and the mutations T366S, L368A and Y407V in the respective other heavy chain (numbering according to Kabat EU index),
j) a full length constant region of the human subclass IgG1 with the mutations L234A, L235A, P329G, I253A, H310A and H435A in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index)
k) a full length constant region of the human subclass IgG1 with the mutations L234A, L235A, P329G, M252Y, S254T and T256E in both heavy chains and the mutation T366W in one heavy chain and the mutations T366S, L368A and Y407V in the respective other heavy chain (numbering according to Kabat EU index)
l) a full length constant region of the human subclass IgG1 with the mutations L234A, L235A, P329G, M252Y, S254T and T256E in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index)
m) a full length constant region of the human subclass IgG1 with the mutations L234A, L235A, P329G, H310A, H433A and Y436A in both heavy chains and the mutation T366W in one heavy chain and the mutations T366S, L368A and Y407V in the respective other heavy chain (numbering according to Kabat EU index), or
n) a full length constant region of the human subclass IgG1 with the mutations L234A, L235A, P329G, H310A, H433A and Y436A in both heavy chains and the mutations i) T366W, and ii) S354C or Y349C, in one heavy chain and the mutations i) T366S, L368A, and Y407V, and ii) Y349C or S354C, in the respective other heavy chain (numbering according to Kabat EU index).

In one aspect, additionally the C-terminal glycine (Gly446) of the heavy chain constant region or CH3 domain is present. In one aspect, additionally the C-terminal glycine (Gly446) and the C-terminal lysine (Lys447) is present (numbering according to Kabat).

In a further aspect, an anti-LNA antibody according to any of the above aspects may incorporate any of the features, singly or in combination, as described in Sections 1-8 below: 1. Antibody Affinity In certain aspects, an antibody provided herein has a dissociation constant (KD) of <1 μM, <100 nM, <10 nM, <1 nM, <0.1 nM, <0.01 nM, or <0.001 nM (e.g., 10⁻⁸ M or less, e.g., from 10⁻⁸ M to 10¹³ M, e.g., from 10⁻⁹ M to 10⁻¹³ M).

In more detail, LNA binding of the antibodies according to the current invention was characterized by surface plasmon resonance (SPR) using a BIAcore instrument with a HEPES-based running and dilution buffer at 25° C. About 100-200 resonance units (RU) of an anti-LNA antibody with an Fc-region of the human IgG1 subclass and the mutations P329G, L234A and L235A (according to Kabat EU index; human IgG Fc-PGLALA) or an anti-LNA Fab fragment with human CL and CH1 (kappa, IgG1 isotype), respectively, was captured on the surface of a Series S Sensor Chip CM3 derivatized by standard amine coupling with either a mouse anti-human IgG Fc-PGLALA antibody or a goat anti-human F(ab′2) antibody with final total surface densities of approximately 5000 RU. Different LNA-modified ASOs were passed over the derivatized SPR chip surface by injecting the respective solutions in a 1:3 dilution series up to a final concentration of 1000 nM. Association was monitored for 3 min and dissociation for 5 min at a flow rate of 30 μl/min each. Bulk refractive index differences were corrected by subtracting blank injections and by subtracting the response obtained from the reference flow cell without captured antibody. Curve fitting was performed using the 1:1 Langmuir binding model within the BIAcore evaluation software.

### 2. Antibody Fragments

In certain aspects, an antibody provided herein is an antibody fragment.

In one aspect, the antibody fragment is a Fab, Fab′, Fab′-SH, or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab’ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc-region. For discussion of Fab and F(ab′)2 fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404 097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CHT-linker-VL-CL, b) VL-CL-linker-VH-CHT, c) VH-CL-linker-VL-CHT or d) VL-CHT-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody.

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody.

Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region.

In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); US Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for LNA and the other specificity is for any other antigen. In certain aspects, bispecific antibodies may bind to two (or more) different epitopes of LNA. Multispecific (e.g., bispecific) antibodies may also be used to localize cytotoxic agents or cells to cells which express LNA. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” comprising an antigen binding site that binds to LNA as well as another different antigen, or two different epitopes of LNA (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for LNA and the other is for CD3.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp. Cell Res. 317, 1255-1260 (2011)); diabodies (Holliger et al., Prot. Eng. 9, 299-305 (1996)) and derivatives thereof, such as tandem diabodies (“TandAb”; Kipriyanov et al., J. Mol. Biol. 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 5. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 2-2 under the heading of “preferred substitutions”. More substantial changes are provided in Table 2-2 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2-2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In certain aspects, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in the HVRs. Such alterations may, for example, be outside of antigen contacting residues in the HVRs. In certain variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc-region, the oligosaccharide attached thereto may be altered. Native antibodies produced by mammalian cells typically comprise a branched, biantennary oligosaccharide that is generally attached by an N-linkage to Asn297 of the CH2 domain of the Fc-region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc-region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc-region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc-region (EU numbering of Fc-region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc-region may have improved FcγRIIIA receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng. 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc-region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotech Bioeng 93, 851-861 (2006); WO 99/54342; WO 2004/065540, WO 2003/011878.

Antibody variants with at least one galactose residue in the oligosaccharide attached to the Fc-region are also provided. Such antibody variants may have improved CDC function. Such antibody variants are described, e.g., in WO 1997/30087; WO 1998/58964; and WO 1999/22764.

### c) Fc-Region Variants

In certain aspects, one or more amino acid modifications may be introduced into the Fc-region of an antibody provided herein, thereby generating an Fc-region variant. The Fc-region variant may comprise a human Fc-region sequence (e.g., a human IgG1, IgG2, IgG3 or IgG4 Fc-region) comprising an amino acid modification (e.g., a substitution) at one or more amino acid positions.

In certain aspects, the invention contemplates an antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc-region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc-region with one or more amino acid substitutions which improve ADCC, e.g., substitutions at positions 298, 333, and/or 334 of the Fc-region (EU numbering of residues).

In certain aspects, an antibody variant comprises an Fc-region with one or more amino acid substitutions which diminish FcγR binding, e.g., substitutions at positions 234 and 235 of the Fc-region (EU numbering of residues). In one aspect, the substitutions are L234A and L235A (LALA). In certain aspects, the antibody variant further comprises D265A and/or P329G in an Fc-region derived from a human IgG1 Fc-region. In one aspect, the substitutions are L234A, L235A and P329G (LALA-PG) in an Fc-region derived from a human IgG1 Fc-region. (See, e.g., WO 2012/130831). In another aspect, the substitutions are L234A, L235A and D265A (LALA-DA) in an Fc-region derived from a human IgG1 Fc-region.

In some aspects, alterations are made in the Fc-region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc-region with one or more substitutions therein which improve binding of the Fc-region to FcRn. Such Fc variants include those with substitutions at one or more of Fc-region residues: 238, 252, 254, 256, 265, 272, 286, 303, 305, 307, 311, 312, 317, 340, 356, 360, 362, 376, 378, 380, 382, 413, 424 or 434, e.g., substitution of Fc-region residue 434 (See, e.g., U.S. Pat. No. 7,371,826; Dall'Acqua, W. F., et al. J. Biol. Chem. 281 (2006) 23514-23524).

Fc-region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc-region with one or more amino acid substitutions, which reduce FcRn binding, e.g., substitutions at positions 253, and/or 310, and/or 435 of the Fc-region (EU numbering of residues). In certain aspects, the antibody variant comprises an Fc-region with the amino acid substitutions at positions 253, 310 and 435. In one aspect, the substitutions are I253A, H310A and H435A in an Fc-region derived from a human IgG1 Fc-region. See, e.g., Grevys, A., et al., J. Immunol. 194 (2015) 5497-5508.

In certain aspects, an antibody variant comprises an Fc-region with one or more amino acid substitutions, which reduce FcRn binding, e.g., substitutions at positions 310, and/or 433, and/or 436 of the Fc-region (EU numbering of residues). In certain aspects, the antibody variant comprises an Fc-region with the amino acid substitutions at positions 310, 433 and 436. In one aspect, the substitutions are H310A, H433A and Y436A in an Fc-region derived from a human IgG1 Fc-region. (See, e.g., WO 2014/177460 A1).

In certain aspects, an antibody variant comprises an Fc-region with one or more amino acid substitutions which increase FcRn binding, e.g., substitutions at positions 252, and/or 254, and/or 256 of the Fc-region (EU numbering of residues). In certain aspects, the antibody variant comprises an Fc-region with amino acid substitutions at positions 252, 254, and 256. In one aspect, the substitutions are M252Y, S254T and T256E in an Fc-region derived from a human IgG1 Fc-region. See also Duncan & Winter, Nature 322:738-40 (1988); U.S. Pat. Nos. 5,648,260; 5,624,821; and WO 94/29351 concerning other examples of Fc-region variants. The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions). [[Adapt as appropriate based on full length sequences of the invention]]

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016/040856.

### e) Antibody Derivatives

In certain aspects, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 6. Immunoconjugates

The invention also provides immunoconjugates comprising an anti-LNA antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, min), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### Binding Characteristics of the LNA Binding Antibodies According to the Invention

The LNA-specific antibodies according to the current invention were further analyzed with respect to their binding and epitope characteristics.

The epitope was mapped using immunoassays with capture molecules containing the following binding epitopes as well as combinations thereof:
LNA (2′-0,4-C-methylene),
phosphorothioate in the NA backbone,
nucleotides/sequence of bases,
DNA,
GalNAc-C6-modification.

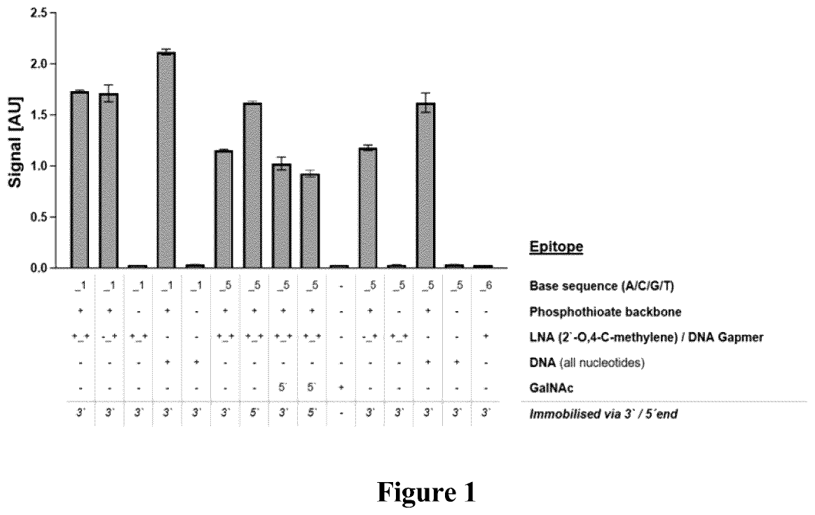

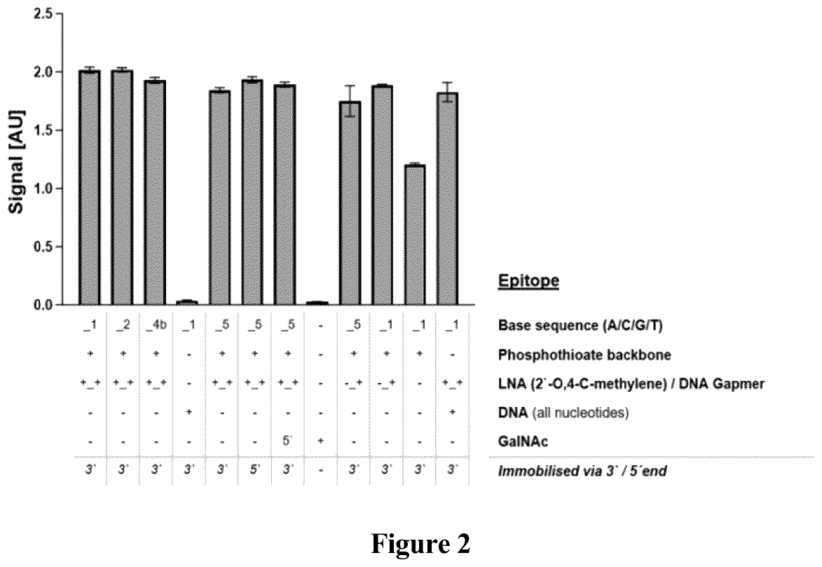

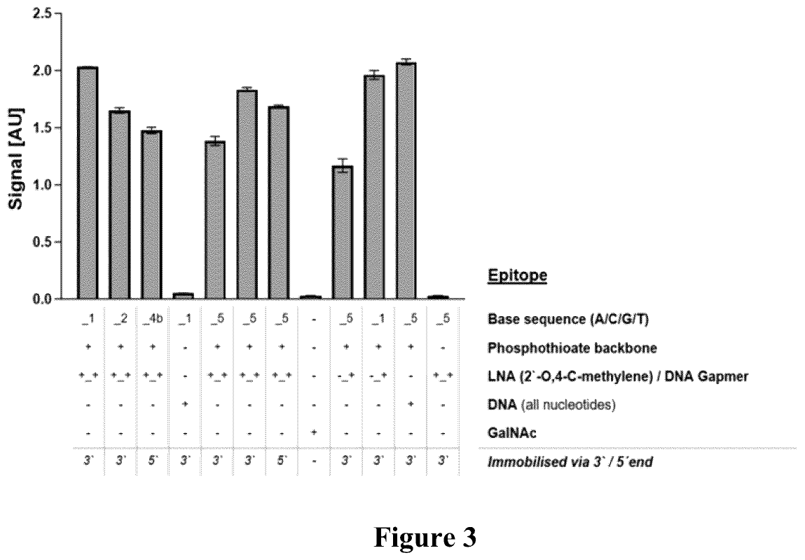

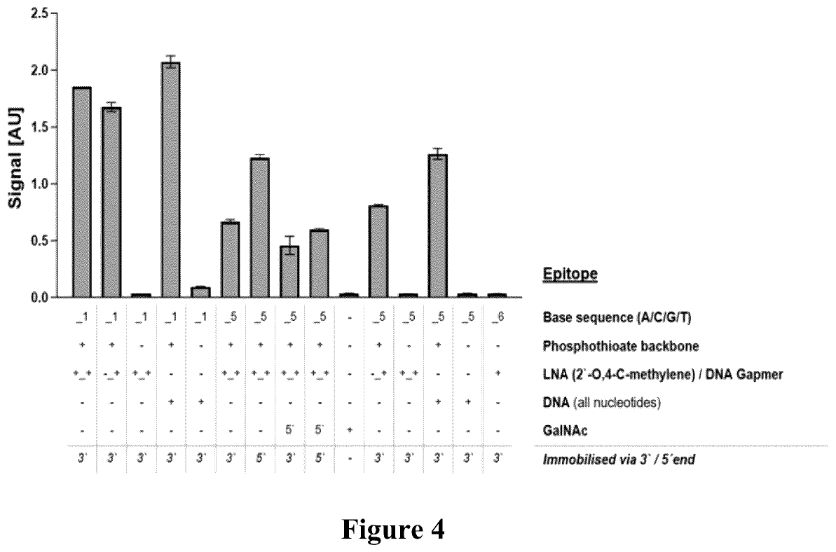

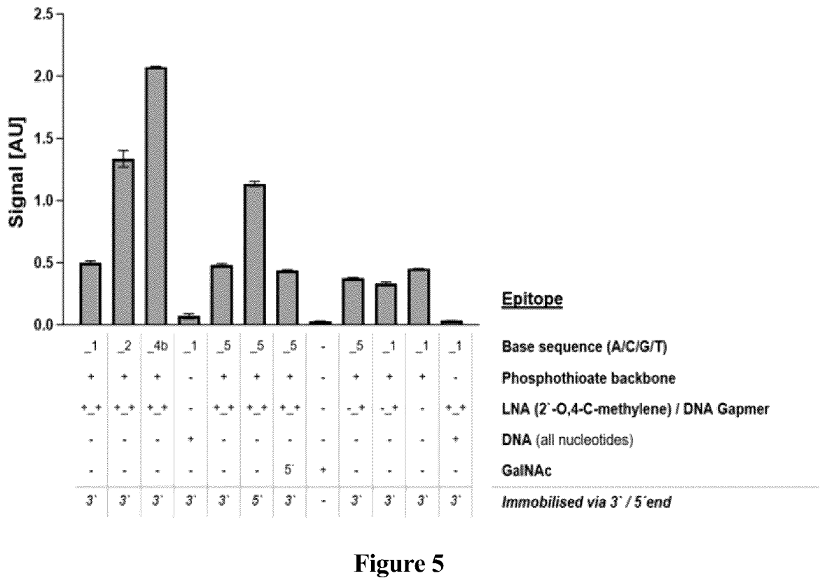

The results of the epitope characterization of the antibodies according to the current invention by solid phase immunoassay (ELISA) using different biotinylated LNA, PS-backbone or DNA-containing NA-oligonucleotides as outlined above are shown in FIGS. 1 to 5. Binding properties as determined in the immunoassays as outlined above are summarized in the following Table 3-1.

TABLE 3-1
Epitope mapping and immunoassay results - epitope binding characteristics.

| | LNA (2′- | Phosphoro- | Impact | | | |
|---|---|---|---|---|---|---|
| | O,4-C- | thioate | of LNA | | GalNAc-C6- | Test system |
| antibody | methylene) | Backbone | sequence | DNA | modification | checked for |
| pAb<GalNAc- | ++ | +++ | −/+ | − | +++ | LBA |
| LNA> | | | | | | immunoassay/ |
| | | | | | | ADA assay/ |
| | | | | | | IF assay |
| pAb<LNA> | + | +++ | + | − | − | LBA |
| | | | | | | immunoassay/ |
| | | | | | | ADA assay |
| pAb<1/2/4b> | + | +++ | + | + | − | ADA assay |
| monoclonal | − | +++ | − | − | − | LBA |
| antibody | | | | | | immunoassay |
| produced by | | | | | | |
| clone 1.2.8 | | | | | | |
| monoclonal | − | +++ | + | − | − | LBA |
| antibody | | | | | | immunoassay |
| produced by | | | | | | |
| clone 1.4.10 | | | | | | |
| monoclonal | − | +++ | −/+ | − | − | LBA |
| antibody | | | | | | immunoassay |
| produced by | | | | | | |
| clone 1.5.13 | | | | | | |
| monoclonal | − | +++ | +++ | − | − | LBA |
| antibody | | | | | | immunoassay |
| produced by | | | | | | |
| clone 1.7.15 | | | | | | |
| monoclonal | ++ | +++ | +++ | − | − | LBA |
| antibody | | | | | | immunoassay |
| produced by | | | | | | |
| clone 1.9.21 | | | | | | |

LBA = ligand binding;
ADA = anti-drug antibody;
IF = immunofluorescence
Reactivity to the depicted epitopes was interpreted by the signal-to-noise (S/N) ratio of the signals of the corresponding immunoassay:
−: S/N <2;
−/+: S/N: 2 to 3;
+: S/N: 3 to 5;
++: S/N: 5 to 10;
+++: S/N ≥10

From the data in Table 3-1 it can be seen that all exemplary antibodies according to the current invention specifically bind to the phosphorothioate backbone of LNA, especially with a signal to noise ratio of 10 or more. This is one preferred embodiment of all aspects of the current invention.

Further, it can be seen that the monoclonal antibodies produced by clones 1.7.15 and 1.9.21 are LNA sequence specific binders. This is one preferred embodiment of said antibodies.

Also, it can be seen that monoclonal antibody produced by clone 1.9.21 specifically binds to LNA (2′-0,4-C-methylene), especially with a signal to noise ratio of 5 to 10. This is one preferred embodiment of said antibody.

To assess the binding specificities and affinities of the LNA-binding antibodies according to the current invention, surface plasmon resonance was used as described, e.g., in WO 2020/141117 and WO 2022/175217.

In more detail, LNA binding of the antibodies according to the current invention was characterized by surface plasmon resonance (SPR) using a BIAcore instrument with a HEPES-based running and dilution buffer at 25° C. About 100-200 resonance units (RU) of an anti-LNA antibody with an Fc-region of the human IgG1 subclass and the mutations P329G, L234A and L235A (according to Kabat EU index; human IgG Fc-PGLALA) or an anti-LNA Fab fragment with human CL and CH1 (kappa, IgG1 isotype), respectively, was captured on the surface of a Series S Sensor Chip CM3 derivatized by standard amine coupling with either a mouse anti-human IgG Fc-PGLALA antibody or a goat anti-human F(ab′2) antibody with final total surface densities of approximately 5000 RU. Different LNA-modified ASOs were passed over the derivatized SPR chip surface by injecting the respective solutions in a 1:3 dilution series up to a final concentration of 1000 nM. Association was monitored for 3 min and dissociation for 5 min at a flow rate of 30 μl/min each. Bulk refractive index differences were corrected by subtracting blank injections and by subtracting the response obtained from the reference flow cell without captured antibody. Curve fitting was performed using the 1:1 Langmuir binding model within the BIAcore evaluation software.

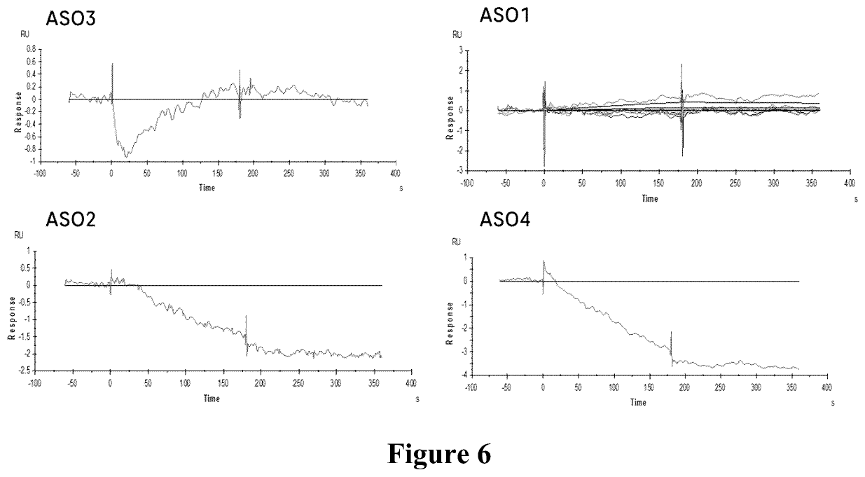

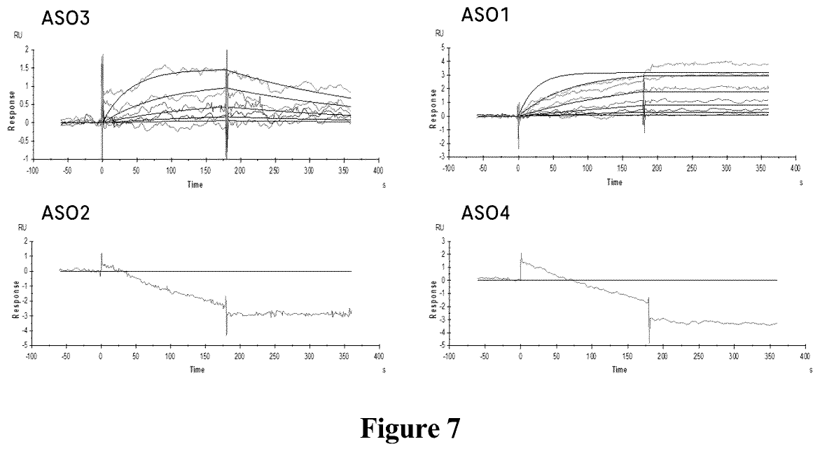

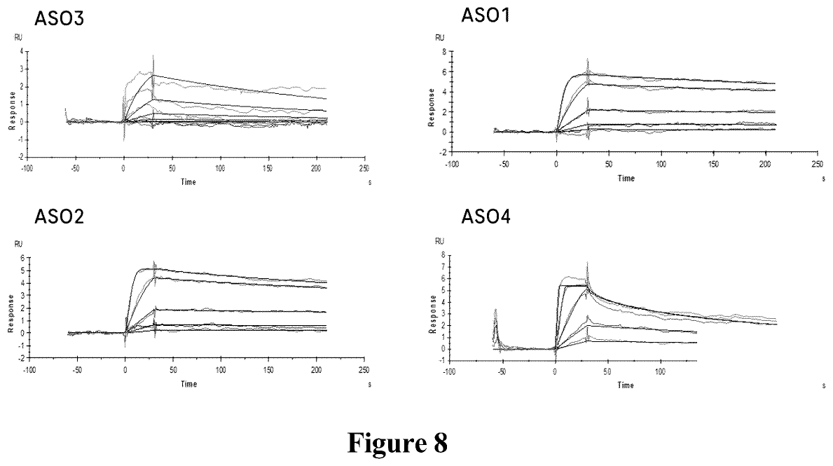

The binding kinetics and affinities of three anti-LNA antibodies according to the current invention, i.e. the anti-LNA antibodies according to the current invention produced by clone 1.2.8 (mAb 5391), 1.7.15 (mAb 5392) and 1.9.21 (mAb 5393), were determined in the bivalent standard Y-shaped IgG format. The results of these analyses are shown in Table 3-2 and example sensorgrams are shown in FIGS. 6 to 8.

TABLE 3-2
Binding kinetics and affinities of the anti-LNA antibodies according to the
current invention produced by clone 1.2.8 (mAb 5391), 1.7.15 (mAb 5392)
and 1.9.21 (mAb 5393) in the bivalent standard Y-shaped IgG format.

| | | ASO composition | [1/Ms] | Koff | K | molar |
|---|---|---|---|---|---|---|
| mAb | analyte | or conjugate structure | Kon | [1/s] | [M] | ratio |
| mAb | ASO 420; SEQ ID | T*T*A*A*c*t*c*a | no binding | | | |
| 5391 | NO: 139 | *a**t*c*a*a*t*t*C | | | | |
| | | *T*C*A | | | | |
| | ASO 827; SEQ ID | C*T*T*T*a*a*t*t* | no binding | | | |
| | NO: 140 | t*a*a*t*c*a*c*t*C | | | | |
| | | *A*T | | | | |
| | ASO 297; SEQ ID | G*A*G*t*t*a*c*t* | no binding | | | |
| | NO: 141 | t*g*c*c*a*A*C*T | | | | |
| | ASO 042; SEQ ID | C*c*T*t*t*C*a*c* | no binding | | | |
| | NO: 142 | T*c*G*t*T*t*c*c* | | | | |
| | | A*G | | | | |
| | fusion 2489 | IgG1 Fc-region | no/low binding | | | |
| | | with anti-TfR | | | | |
| | | fab 1026 (SEQ ID | | | | |
| | | NO: 143 and 144) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |
| | fusion 2899 | IgG1 Fc-region | no/low binding | | | |
| | | with anti-TfR | | | | |
| | | fab 8D3 (SEQ ID | | | | |
| | | NO: 146 and 147) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |
| mAb | ASO 420; SEQ ID | T*T*A*A*c*t*c*a | 2.14 | 4.23 | 1.98E−07 | 0.2 |
| 5392 | NO: 139 | *a**t*c*a*a*t*t*C | E+04 | | | |
| | | *T*C*A | | | | |
| | ASO 827; SEQ ID | C*T*T*T*a*a*t*** | no binding | | | |
| | NO: 140 | t*a*a*t*c*a*c*t*C | | | | |
| | | *A*T | | | | |
| | ASO 297; SEQ ID | G*A*G*t*t*a*c*** | 1.40 | oos | — | 0.4 |
| | NO: 141 | t*g*c*c*a*A*C*T | E+05 | | | |
| | ASO 042; SEQ ID | C*c*T*t*t*C*a*c* | no binding | | | |
| | NO: 142 | T*c*G*t*T*t*c*c* | | | | |
| | | A*G | | | | |
| | fusion 2489 | IgG1 Fc-region | no/low binding | | | |
| | | with anti-TfR | | | | |
| | | fab 1026 (SEQ ID | | | | |
| | | NO: 143 and 144) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |
| | fusion 2899 | IgG1 Fc-region | no/low binding | | | |
| | | with anti-TfR | | | | |
| | | fab 8D3 (SEQ ID | | | | |
| | | NO: 146 and 147) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |
| mAb | ASO 420; SEQ ID | T*T*A*A*c*t*c*a | 2.81 | 1.06 | 3.79E−11 | 1.1 |
| 5393 | NO: 139 | *a**t*c*a*a*t*t*C | E+07 | E−03 | | |
| | | *T*C*A | | | | |
| | ASO 827; SEQ ID | C*T*T*T*a*a*t*** | 4.26 | 1.76 | 4.14E−11 | 1.0 |
| | NO: 140 | t*a*a*t*c*a*c*t*C | E+07 | E−03 | | |
| | | *A*T | | | | |
| | ASO 297; SEQ ID | G*A*G*t*t*a*c*** | 1.47 | 3.86 | 2.62E−08 | 0.4 |
| | NO: 141 | t*g*c*c*a*A*C*T | E+05 | E−03 | | |
| | ASO 042; SEQ ID | C*c*T*t*t*C*a*c* | oos | 6.45 | <1E−09 | 0.9 |
| | NO: 142 | T*c*G*t*T*t*c*c* | | E−02 | | |
| | | A*G | | | | |
| | fusion 2489 | IgG1 Fc-region | 1.54 | 1.78 | 1.16E−10 | 0.6 |
| | | with anti-TfR | E+07 | E−03 | | |
| | | fab 1026 (SEQ ID | | | | |
| | | NO: 143 and 144) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |
| | fusion 2899 | IgG1 Fc-region | 1.42 | 2.40 | 1.69E−10 | 0.5 |
| | | with anti-TfR | E+07 | E−03 | | |
| | | fab 8D3 (SEQ ID | | | | |
| | | NO: 146 and 147) | | | | |
| | | fused to the N- | | | | |
| | | terminus of one Fc- | | | | |
| | | region polypeptide | | | | |
| | | with ASO 918 | | | | |
| | | (SEQ ID NO: 127) | | | | |
| | | conjugated to | | | | |
| | | position 297 | | | | |
| | | (inserted tag of | | | | |
| | | SEQ ID NO: 143; | | | | |
| | | Kabat EU index) | | | | |
| | | via a linker using | | | | |
| | | KTG | | | | |
| | | transglutaminase | | | | |

uppercase = LNA; oos = out of specification; *= phosphorothioate bond;
#= steady state

MAb 5391 shows no to very low binding to all of the analytes tested. MAb 5392 binds to some of the ASOs, while showing no or very low binding to other ASOs. However, mAb 5393 binds to all of the tested ASOs and antibody-ASO conjugates with good affinity.

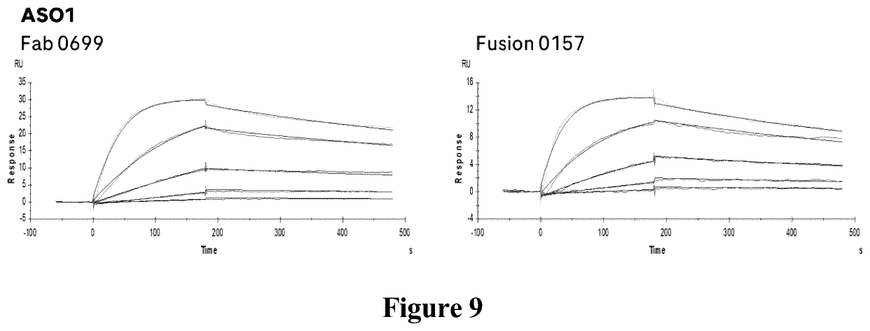

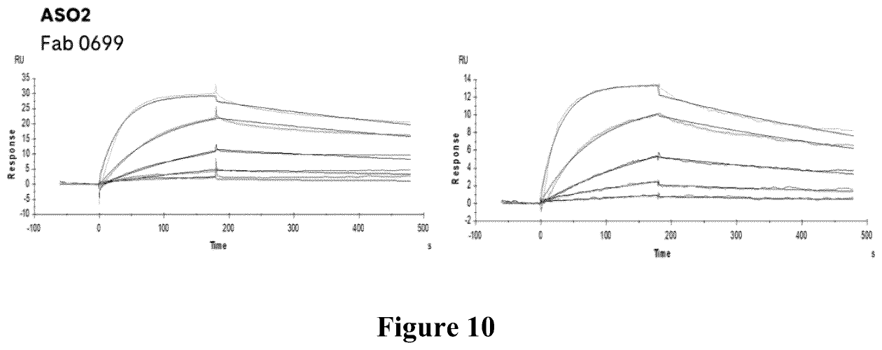

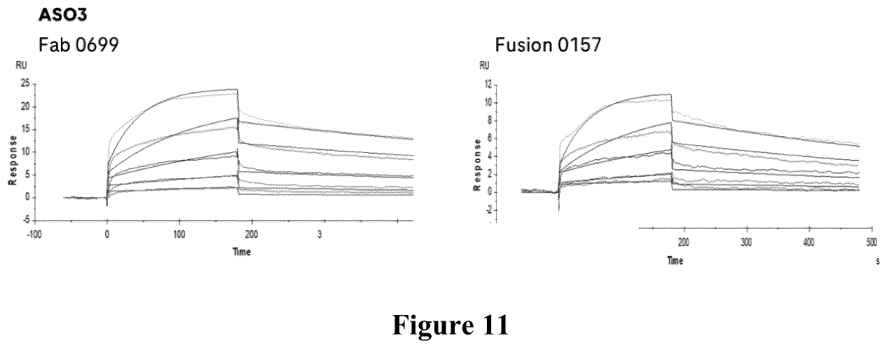

To further characterize mAb 5393 and to avoid avidity effects due to the bivalency, mAb 5393 was produced as monovalent Fab (VH and VL from antibody produced by clone 1.9.21 as Fab; fab 0699; SEQ ID NO: 130 and 131) and as monovalent C-terminal Fc-region Fab fusion molecule (fusion of fab 0699 to one C-terminus of an Fc-region of the IgG1 subclass; fusion 0157). The binding kinetics and affinities towards different LNA-modified ASOs as well as one siRNA (siRNA 664; SEQ ID NO: 128) were tested. The results are summarized in Table 3-3 and example sensorgrams are shown in FIGS. 9 to 12.

TABLE 3-3
Binding kinetics and affinities of the anti-LNA antibody according
to the current invention produced by clone 1.9.21 in Fab (Fab 0669) and Fc-fusion
(Fusion 0157) in monovalent format.

| mole- | | ASO composition or | [1/Ms] | Koff | kD | molar |
|---|---|---|---|---|---|---|
| cule | analyte | conjugate structure | Kon | [1/s] | [M] | ratio |
| fab | ASO 420; | T*T*A*A*c*t*c*a*a ** | 3.5 | 1.1 | 3.0 | — |
| 0699 | SEQ ID | t*c*a*a*t*t*C*T*C*A | E+06 | E−03 | E−10 | |
| | NO: 139 | | | | | |
| | ASO 827; | C*T*T*T*a*a*t*t*t*a* | 2.9 | 1.1 | 4.0 | |
| | SEQ ID | a*t*c*a*c*t*C*A*T | E+06 | E−03 | E−10 | |
| | NO: 140 | | | | | |
| | ASO 297; | G*A*G*t*t*a*c*t*t*g* | 2.2 | 1.1 | 5.1 | |
| | SEQ ID | c*c*a*A*C*T | E+04 | E−03 | E−08 | |
| | NO: 141 | | | | | |
| | ASO 042; | C*c*T*t*t*C*a*c*T*c | | | 1.0 | |
| | SEQ ID | *G*t*T*t*c*c*A*G | | | E−08# | |
| | NO: 142 | | | | | |
| | SiRNA 664 | sense (SEQ ID NO: | | no | binding | |
| | | 128): | | | | |
| | | u*c*ucguggccuuaaugaa | | | | |
| | | *a*t | | | | |
| | | antisense (SEQ ID NO: | | | | |
| | | 129): | | | | |
| | | u*u*ucauuaaggccacgag | | | | |
| | | a*u*u | | | | |
| fusion | ASO 420; | T*T*A*A*c*t*c*a*a** | 3.7 | 1.4 | 4.0 | |
| 0157 | SEQ ID | t*c*a*a*t*t*C*T*C*A | E+06 | E−03 | E−10 | |
| | NO: 139 | | | | | |
| | ASO 827; | C*T*T*T*a*a*t*t*t*a* | 3.2 | 1.6 | 5.0 | |
| | SEQ ID | a*t*c*a*c*t*C*A*T | E+06 | E−03 | E−10 | |
| | NO: 140 | | | | | |
| | ASO 297; | G*A*G*t*t*a*c*t*t*g* | 2.0 | 1.5 | 7.6 | |
| | SEQ ID | c*c*a*A*C*T | E+04 | E−03 | E−08 | |
| | NO: 141 | | | | | |
| | ASO 042; | C*c*T*t*t*C*a*c*T*c | | | 9.0 | |
| | SEQ ID | *G*t*T*t*c*c*A*G | | | E−09 | |
| | NO: 142 | | | | | |
| | siRNA 664 | sense (SEQ ID NO: | | no | binding | |
| | | 128): | | | | |
| | | u*c*ucguggccuuaaugaa | | | | |
| | | *a*t | | | | |
| | | antisense (SEQ ID NO: | | | | |
| | | 129): | | | | |
| | | u*u*ucauuaaggccacgag | | | | |
| | | a*u*u | | | | |

uppercase = LNA; underline = 2′-F; oos = out of specification;
*= phosphorothioate bond; #= steady state

Confirming the results obtained from the bivalent IgG format, anti-LNA antibody according to the current invention produced by clone 1.9.21 in monovalent formats binds with good and comparable affinity to all of the tested single-stranded LNA-modified ASOs, irrespective of defined composition and sequences. However, it does not bind to siRNA, which is double-stranded and not LNA-modified. This is one preferred embodiment of said antibody.

Further analyses of the pan anti-LNA antibody according to the invention produced by clone 1.9.21 a trivalent, bispecific format was used. This trivalent, bispecific format comprises an IgG1 Fc-region (with knobs-into-holes-cys1 mutations) to which at one of the N-termini of the Fc-region a germline Fab (DP47; SEQ ID NO: 137 and 138) has been fused and at both of the C-termini of the Fc-region each one anti-LNA Fab (fab 0699) according to the current invention produced by clone 1.9.21 has been fused (fusion 1861). Using this trivalent, bispecific format an 1:1 antibody-to-analyte binding (antibody binding site:LNA-modified ASO) ratio was confirmed. The respective kD value was in the single digit pico-molar range (Table 3-4).

TABLE 3-4
Binding kinetics and affinities of the anti-LNA antibody according to the
current invention produced by clone 1.9.21 in bispecific trivalent format
(fusion 1861).

| mole- | | ASO composition or | [1/Ms] | Koff | kD | molar |
|---|---|---|---|---|---|---|
| cule | analyte | conjugate structure | Kon | [1/s] | [M] | ratio |
| fusion | ASO | T*T*A*A*c*t*c*a*a** | 4.50 | 2.06 | 4.56 | 1 |
| 1861 | 420; | t*c*a*a*t*t*C*T*C*A | E+07 | E−04 | E−12 | |
| | SEQ ID | | | | | |
| | NO: 139 | | | | | |
| | ASO | C*T*T*T*a*a*t*t*t*a* | 7.84 | 2.38 | 3.04 | 1 |
| | 827; | a*t*c*a*c*t*C*A*T | E+07 | E−04 | E−12 | |
| | SEQ ID | | | | | |
| | NO: 140 | | | | | |
| | ASO | G*A*G*t*t*a*c*t*t*g* | 6.78 | 1.15 | 1.69 | 1 |
| | 297; | c*c*a*A*C*T | E+06 | E−03 | E−10 | |
| | SEQ ID | | | | | |
| | NO: 141 | | | | | |
| | ASO | C*c*T*t*t*C*a*c*T*c | 8.95 | 3.02 | 3.37 | 1 |
| | 042; | *G*t*T*t*c*c*A*G | E+07 | E−04 | E−12 | |
| | SEQ ID | | | | | |
| | NO: 142 | | | | | |

uppercase = LNA; *= phosphorothioate bond

This indicates that LNA-modified ASOs are bound in an avidity-driven manner, i.e. two binding arms of the anti-LNA antibody according to the current invention can bind to one, i.e. the same, LNA-modified ASOs. Thus, LNA-modified ASOs of sufficient lengths can be bound by more than one LNA binding site of the same antibody showing the true sequence independence of the binding of the anti-LNA antibody produced by clone 1.9.21.

To further characterize the binding properties of the anti-LNA antibody according to the current invention produced by clone 1.9.21 in Fab format, the binding to tetramer LNA-modified ASOs that contain various modifications (LNA and phosphorothioate bond) and/or sequences at different positions. The results of these analyses are listed in Table 3-5.

Thus, the anti-LNA antibodies according to the current invention and especially the anti-LNA antibody produced by clone 1.9.21 have the following binding characteristics:
non-LNA-modified ASO is not bound, at least one phosphorothioate bond or one LNA-modified sugar must be present
1^{st} base preferentially to be C or T or G=not A
LNA-modified sugar preferable at the 2nd position, i.e. if only 1 or 2 LNA (out of 4) are presence, then the 2nd position must be a LNA (otherwise no binding), if the 2nd position is not a LNA but 1st, 3rd and 4th are all LNA, there can be week binding (affinity ~100× less)
more LNA-modification correlates with higher affinity
phosphorothioate bond located only between the second and the third nucleotide is not preferred, i.e. the binding is too weak for the intended use or is even completely abolished.

TABLE 3-5
Binding of the anti-LNA antibody according to the current invention
produced by clone 1.9.21 in Fab format to tetramer LNA-modified ASOs
that contain various modifications (LNA and phosphorothioate bond)
and/or sequences at different positions.

| mole- | tetramer | | Kon | Koff | kD |
|---|---|---|---|---|---|
| cule | analyte | composition | [1/Ms] | [1/s] | [M] |
| fab | TCAC | UpUpUpU | no binding | | |
| 0699 | TCAC | UsLsUsU | 1.46E+05 | 8.88E−03 | 6.07E−08 |
| | CCAC | UsLsUsU | 3.23E+04 | 3.56E−03 | 1.10E−07 |
| | ACAC | UsLsUsU | 5.14E+03 | 1.43E−02 | 2.78E−06 |
| | GCAC | UsLsUsU | 1.42E+04 | 1.25E−03 | 8.76E−08 |
| | TCAC | UpLpUpU | 9.76E+03 | 1.18E−03 | 1.21E−07 |
| | TCAC | UsLpUpU | 2.96E+04 | 1.06E−03 | 3.57E−08 |
| | TCAC | UpLsUpU | no binding | | |
| | TCAC | UpLpUsU | 2.73E+04 | 1.06E−03 | 3.88E−08 |
| | TCAC | UsLsUpU | 4.13E+04 | 1.38E−03 | 3.34E−08 |
| | TCAC | UpLsUsU | 5.79E+03 | 1.84E−03 | 3.17E−07 |
| | TCAC | UsLpUsU | 3.51E+04 | 2.02E−03 | 5.77E−08 |
| | TCAC | UsUsUsU | 1.18E+04 | 1.58E−03 | 1.34E−07 |
| | TCAC | LsUsUsU | no binding | | |
| | TCAC | UsUsLsU | 1.51E+04 | 1.96E−03 | 1.30E−07 |
| | TCAC | UsUsUsL | 4.09E+02 | 2.14E−02 | 5.23E−05 |
| | TCAC | LsLsUsU | 4.02E+05 | 2.70E−02 | 6.73E−08 |
| | TCAC | UsLsLsU | irregular binding events | | |
| | TCAC | UsUsLsL | no binding | | |
| | TCAC | LsUsLsU | no binding | | |
| | TCAC | LsUsUsL | no binding | | |
| | TCAC | UsLsUsL | 2.69E+04 | 7.84E−03 | 2.92E−07 |
| | TCAC | LsLsLsU | 5.39E+06 | 1.86E−02 | 3.45E−09 |
| | TCAC | LsLsUsL | 9.80E+03 | 3.76E−04 | 3.84E−08 |
| | TCAC | LsUsLsL | 5.85E+04 | 2.04E−01 | 3.49E−06 |
| | TCAC | UsLsLsL | 3.05E+04 | 2.27E−03 | 7.43E−08 |
| | TCAC | LsLsLsL | 2.57E+06 | 6.46E−03 | 2.51E−09 |

U= unmodified, L= locked, p=phosphodiester, s= phosphorothioate

Thus, in one aspect, the invention provides antibodies that bind to LNA-modified nucleic acids. In one aspect, provided are isolated antibodies that bind to LNA. In one aspect, the invention provides antibodies that specifically bind to LNA. In certain aspects, an anti-LNA antibody according to the current invention
binds to a nucleic acid comprising at least one phosphorothioate bond or one LNA-modified sugar must be present;
shows significantly reduced or no binding to a nucleic acid without an LNA-modification, i.e. in case all phosphorothioate bonds are presence, it can still bind to nucleic acid without any LNA but with reduced affinity (~10× weaker);
preferably binds to a nucleic acid comprises as 1^{st} base (5′-base) C or T or G but not A;
binds preferably to a nucleic acid, wherein the LNA-modified sugar is at the 2nd position counted from the 5′-end;
binds sequence independent to the LNA-modification;
can bind to more than one LNA-modification in the same nucleic acid with different binding sites simultaneously, i.e. the binding affinity increases with the number of LNA-modifications and binding sites;
does not preferably bind to nucleic acids with a phosphorothioate bond located only between the second and the third nucleotide.

### Thus, in One Aspect, the Invention Provides an Anti-LNA Antibody Comprising

(a) a HVR-H1 comprising the amino acid sequence of SEQ ID NO: 42;
(b) a HVR-H2 comprising the amino acid sequence of SEQ ID NO: 44; and
(c) a HVR-H3 comprising the amino acid sequence of SEQ ID NO: 46.

### In One Aspect, the Invention Provides an Anti-LNA Antibody Comprising

(d) a HVR-L1 comprising the amino acid sequence of SEQ ID NO: 51;
(e) a HVR-L2 comprising the amino acid sequence of SEQ ID NO: 53; and
(f) a HVR-L3 comprising the amino acid sequence of SEQ ID NO: 55.

### In One Preferred Aspect, the Invention Provides an Anti-LNA Antibody Comprising

(a) a HVR-H1 comprising the amino acid sequence of SEQ ID NO: 42;
(b) a HVR-H2 comprising the amino acid sequence of SEQ ID NO: 44;
(c) a HVR-H3 comprising the amino acid sequence of SEQ ID NO: 46;
(d) a HVR-L1 comprising the amino acid sequence of SEQ ID NO: 51;
(e) a HVR-L2 comprising the amino acid sequence of SEQ ID NO: 53; and
(f) a HVR-L3 comprising the amino acid sequence of SEQ ID NO: 55.

### In Another Aspect, an Anti-LNA Antibody of the Invention Comprises

a VH domain comprising
   (a) a HVR-H1 comprising the amino acid sequence of SEQ ID NO: 42;
   (b) a HVR-H2 comprising the amino acid sequence of SEQ ID NO: 44; and
   (c) a HVR-H3 comprising the amino acid sequence of SEQ ID NO: 46; and
a VL domain comprising
   (d) a HVR-L1 comprising the amino acid sequence of SEQ ID NO: 51;
   (e) a HVR-L2 comprising the amino acid sequence of SEQ ID NO: 53; and
   (f) a HVR-L3 comprising the amino acid sequence of SEQ ID NO: 55.

In any of the aspects provided herein, the preferred anti-LNA antibody according to the invention is a humanized antibody. In one aspect, the preferred anti-LNA antibody according to the invention further comprises besides the HVRs as outlined above an acceptor human framework, e.g. a human immunoglobulin framework or a human consensus framework. Thus, in another aspect, the preferred anti-LNA antibody according to the invention comprises a VH domain comprising a HC-FR1, a HC-FR2, a HC-FR3 and a HC-FR4 each independently of each other of at least 85%, 86%, 87%, 88%, 89%, 90%, 91%, 92%, 93%, 94%, 95%, 96%, 97%, 98% or 99% sequence identity to a respective human germline FR sequence; and a VL domain comprising a LC-FR1, a LC-FR2, a LC-FR3 and a LC-FR4 each independently of each other of at least 85%, 86%, 87%, 88%, 89%, 90%, 91%, 92%, 93%, 94%, 95%, 96%, 97%, 98% or 99% sequence identity to a respective human germline FR sequence.

In another aspect, an anti-LNA antibody according to the invention comprises the three HVR sequences of the VH of SEQ ID NO: 48.

In another aspect, an anti-LNA antibody according to the invention comprises the three HVR sequences of the VL of SEQ ID NO: 57.

In another preferred aspect, an anti-LNA antibody according to the invention comprises the six HVR sequences of the VH and VL of SEQ ID NO: 48 and 57, i.e. the anti-LNA antibody comprises the HVR-H1, HVR-H2 and HVR-H3 amino acid sequences of the VH domain of SEQ ID NO: 48 and the HVR-L1, HVR-L2 and HVR-L3 amino acid sequences of the VL domain of SEQ ID NO: 57.

In another aspect, an anti-LNA antibody according to the invention comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 48.

In one preferred aspect, an anti-LNA antibody according to the invention comprises a heavy chain variable domain (VH) sequence having at least 95%, sequence identity to the amino acid sequence of SEQ ID NO: 48.

In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-LNA antibody comprising that sequence retains the ability to bind to LNA. In certain aspects, a total of 1 to 10 amino acids have been substituted, inserted and/or deleted in SEQ ID NO: 48.

In certain aspects, substitutions, insertions, or deletions occur in regions outside the HVRs (i.e., in the FRs). Optionally, the anti-LNA antibody according to the invention comprises the VH sequence of SEQ ID NO: 48, including post-translational modifications of that sequence.

In another aspect, an anti-LNA antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 57.

In one preferred aspect, an anti-LNA antibody comprises a light chain variable domain (VL) sequence having at least 95% sequence identity to the amino acid sequence of SEQ ID NO: 57.

In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-LNA antibody comprising that sequence retains the ability to bind to LNA. In certain preferred aspects, a total of 1 to 10 amino acids have been substituted, inserted and/or deleted in SEQ ID NO: 57.

In certain aspects, the substitutions, insertions, or deletions occur in regions outside the HVRs (i.e., in the FRs). Optionally, the anti-LNA antibody according to the invention comprises the VL sequence of SEQ ID NO: 57, including post-translational modifications of that sequence.

In another aspect, an anti-LNA antibody is provided, wherein the antibody comprises a VH sequence as in any of the aspects provided above, and a VL sequence as in any of the aspects provided above. In one preferred aspect, the antibody according to the invention comprises the VH and VL sequences of SEQ ID NO: 48 and 57, respectively, including post-translational modifications of those sequences.

In a preferred aspect of the invention, an anti-LNA antibody according to any of the above aspects is a monoclonal antibody, including a chimeric, humanized or human antibody. In one aspect, an anti-LNA antibody is an antibody fragment, e.g., an Fv, Fab, Fab′, scFv, diabody, or F(ab′)2 fragment.

In another aspect, the preferred antibody is a full length antibody, e.g., an intact IgG1 antibody or other antibody class or isotype as defined herein.

In a further aspect, the preferred antibody according to the invention are of IgG1 isotype/subclass and comprise a constant heavy chain region with the amino acid sequence of SEQ ID NO: 90 or one or more domains of the constant heavy chain region with the amino acid sequence of SEQ ID NO: 90.

In a further aspect, the preferred antibody according to the current invention comprise a VH and VL sequence as in any of the above preferred aspects and
a) a full length constant region of the human subclass IgG1, preferably of SEQ ID NO: 90, or
b) a full length constant region of the human subclass IgG4, preferably of SEQ ID NO: 91, or
c) a full length constant region of the human subclass IgG1 with the mutations L234A, L235A and P329G, preferably of SEQ ID NO: 92 (numbering according to Kabat EU index),
d) a full length constant region of the human subclass IgG4 with the mutations S228P and L235E, preferably of SEQ ID NO: 93 (numbering according to Kabat EU index),
e) a full length constant region of the human subclass IgG1 with the mutations L234A, L235A and P329G in both heavy chains and the mutation T366W in one heavy chain and the mutations T366S, L368A and Y407V in the respective other heavy chain, preferably of SEQ ID NO: 94 and 95 (numbering according to Kabat EU index),
f) a full length constant region of the human subclass IgG1 with the mutations L234A, L235A and P329G in both heavy chains and the mutations i) T366W, and ii) S354C or Y349C, in one heavy chain and the mutations i) T366S, L368A, and Y407V, and ii) Y349C or S354C, in the respective other heavy chain, preferably of SEQ ID NO: 96 and 97 or SEQ ID NO: 98 and 99 (numbering according to Kabat EU index),
g) a full length constant region of the human subclass IgG4 with the mutations S228P and L235E in both heavy chains and the mutation T366W in one heavy chain and the mutations T366S, L368A and Y407V in the respective other heavy chain, preferably of SEQ ID NO: 100 and 101 (numbering according to Kabat EU index),
h) a full length constant region of the human subclass IgG4 with the mutations S228P and L235E in both heavy chains and the mutations i) T366W, and ii) S354C or Y349C, in one heavy chain and the mutations i) T366S, L368A, and Y407V, and ii) Y349C or S354C, in the respective other heavy chain, preferably of SEQ ID NO: 102 and 103 or SEQ ID NO: 104 and 105 (numbering according to Kabat EU index),
i) a full length constant region of the human subclass IgG1 with the mutations L234A, L235A, P329G, I253A, H310A and H435A in both heavy chains and the mutation T366W in one heavy chain and the mutations T366S, L368A and Y407V in the respective other heavy chain, preferably of SEQ ID NO: 106 and 107 (numbering according to Kabat EU index),
j) a full length constant region of the human subclass IgG1 with the mutations L234A, L235A, P329G, I253A, H310A and H435A in both heavy chains and the mutations i) T366W, and ii) S354C or Y349C, in one heavy chain and the mutations i) T366S, L368A, and Y407V, and ii) Y349C or S354C, in the respective other heavy chain, preferably of SEQ ID NO: 108 and 109 or SEQ ID NO: 110 and 111 (numbering according to Kabat EU index)
k) a full length constant region of the human subclass IgG1 with the mutations L234A, L235A, P329G, M252Y, S254T and T256E in both heavy chains and the mutation T366W in one heavy chain and the mutations T366S, L368A and Y407V in the respective other heavy chain, preferably of SEQ ID NO: 112 and 113 (numbering according to Kabat EU index)
l) a full length constant region of the human subclass IgG1 with the mutations L234A, L235A, P329G, M252Y, S254T and T256E in both heavy chains and the mutations i) T366W, and ii) S354C or Y349C, in one heavy chain and the mutations i) T366S, L368A, and Y407V, and ii) Y349C or S354C, in the respective other heavy chain, preferably of SEQ ID NO: 114 and 115 or SEQ ID NO: 116 and 117 (numbering according to Kabat EU index)
m) a full length constant region of the human subclass IgG1 with the mutations L234A, L235A, P329G, H310A, H433A and Y436A in both heavy chains and the mutation T366W in one heavy chain and the mutations T366S, L368A and Y407V in the respective other heavy chain, preferably of SEQ ID NO: 118 and 119 (numbering according to Kabat EU index), or
n) a full length constant region of the human subclass IgG1 with the mutations L234A, L235A, P329G, H310A, H433A and Y436A in both heavy chains and the mutations i) T366W, and ii) S354C or Y349C, in one heavy chain and the mutations i) T366S, L368A, and Y407V, and ii) Y349C or S354C, in the respective other heavy chain, preferably of SEQ ID NO: 120 and 121 or SEQ ID NO: 122 and 123 (numbering according to Kabat EU index).

In one aspect, additionally the C-terminal glycine (Gly446) of the heavy chain constant region or CH3 domain is present (numbering according to Kabat). In one aspect, additionally the C-terminal glycine (Gly446) and the C-terminal lysine (Lys447) is present (numbering according to Kabat).

### Application of Anti-LNA Antibodies According to the Invention in Anti-Drug-Antibody (ADA) Assays

ASOs typically differ from their endogenous counterparts by the use of non-natural nucleotides, backbones or carriers. Therefore, e.g., an unwanted immune response to an oligonucleotide therapeutic can be generated in principle to any of the carrier, backbone, oligonucleotide sequence, or any novel epitopes created from the whole drug (carrier plus oligonucleotide). The specific anti-LNA antibodies according to the current invention can mimic such an immune response and therefore are highly suitable and useful, e.g., as a positive control in an assay for determining anti-ASO(LNA)-drug antibodies.

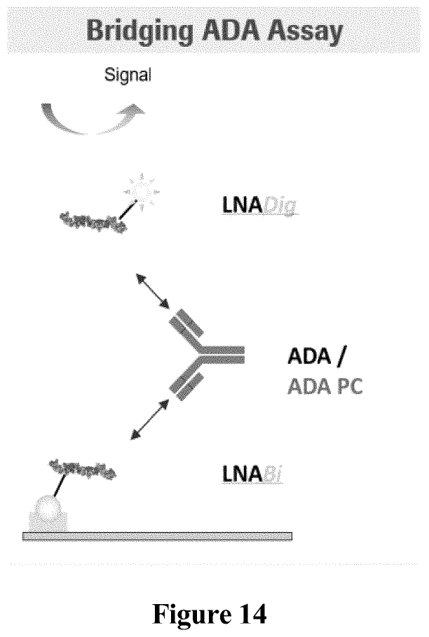

Typical assay formats for detecting LNA-specific ADAs by a bridging (ADA) assay or a direct (ADA) assay are shown in FIG. 14 (bridging ADA assay) and 15 (direct ADA assay).

In one exemplary setup, a mouse-human chimeric version of the LNA-specific mAb according to the current invention produced by clone 1.9.21 (mAb<LNA>Chim-1.9.21-IgG) was used as ADA positive control in an exemplary direct ADA assay.

In more detail, a mixture of two biotinylated drug molecules (5′-Bi-LNA/3′-Bi-LNA) was used as capture moiety in a serial sandwich ELISA. In a first step, a streptavidin (SA) coated microtiter plate (SA-MTP) was contacted with the biotinylated capture molecules 5′-Bi-LNA/3′-Bi-LNA for coating. Test samples, positive control mAb<LNA>Chim-1.9.21-IgG and positive control samples are added to the coated microtiter plate and incubated to immobilize ADA-drug complexes via the biotin-labeled capture molecules. For detection, immobilized ADA-drug complexes are successively incubated with a monoclonal anti-human-IgG-antibody-Dig conjugate (mAb<h-Fc-pan>M-R10Z8E9-IgG-Dig) and a polyclonal anti-digoxygenin Fab fragments conjugated to horseradish peroxidase (anti-digoxygenin-POD (poly)). Finally, the generated peroxidase is visualized by ABTS substrate solution resulting in the formation of a colored reaction product. The color intensity is proportional to the ADA analyte concentration in the plasma sample, e.g. from an experimental animal or of a human individual.

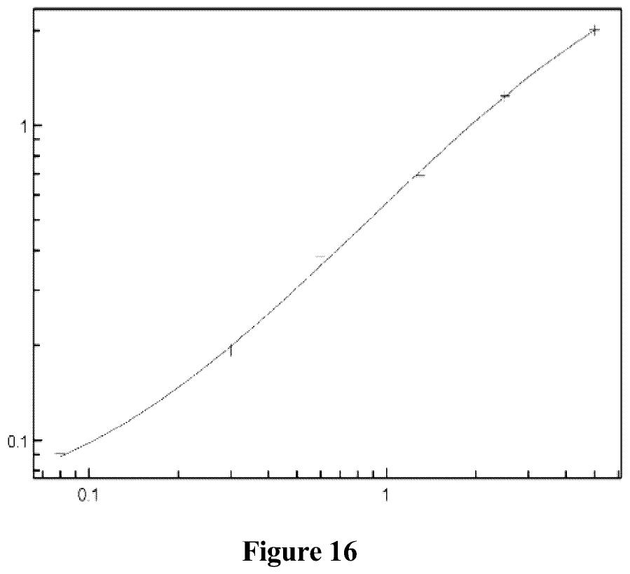

A typical calibration curve of the positive control and corresponding signal data of the assay is shown in FIG. 16 and Table 4-1.

TABLE 4-1
Calibration curve of a direct ADA assay with an antibody according
to the current invention as calibrator/positive control.

| | positive control concentration [ng/mL] | | | | | | | |
|---|---|---|---|---|---|---|---|---|
| | 5.0 | 2.50 | 1.25 | 0.625 | 0.313 | 0.156 | 0.078 | 0.0 |
| mean signal | 2.0045 | 1.2380 | 0.6905 | 0.3825 | 0.1930 | 0.1235 | 0.0910 | 0.0555 |
| [AU] | | | | | | | | |

Thus, one aspect of the current invention is an immunoassay for the determination of the presence or amount of an anti-drug antibody (ADA), wherein the drug comprises an LNA and the ADA is specific for the LNA, wherein the positive control calibration curve for the immunoassay is generated using an antibody according to the current invention in at least two different concentrations.

In one embodiment the immunoassay is a bridging ELISA. In one embodiment the bridging ELISA comprises the steps of
a) immobilizing the LNA-containing drug on a solid surface,
b) incubating the solid surface-immobilized drug obtained in step a) with a sample suspected to comprises ADAs against the LNA-containing drug and thereby obtaining an immobilized drug-ADA complex,
c) determining the presence or amount of the immobilized drug-ADA complex by incubating the surface obtained in step b) with the LNA-containing drug conjugated to a detectable label and correlating the signal of the detectable label to the calibration curve.

In one embodiment the immunoassay is a direct ELISA. In one embodiment the bridging ELISA comprises the steps of
a) immobilizing the LNA-containing drug on a solid surface,
b) incubating the solid surface-immobilized drug obtained in step a) with a sample suspected to comprises ADAs against the LNA-containing drug and thereby obtaining an immobilized drug-ADA complex,
c) determining the presence or amount of the immobilized drug-ADA complex by incubating the surface obtained in step b) with an anti-Fc-region antibody specific for the species of the ADA that is conjugated to a detectable label and correlating the signal of the detectable label to the calibration curve.

Use of an antibody according to the current invention as standard or positive control in an immunoassay.

Use of an antibody according to the current invention in at least two different concentrations for generating a calibration curve for an immunoassay.

In one embodiment the immunoassay is a bridging ELISA or a direct ELISA.

In one embodiment the immunoassay is for the determination of the presence or amount of an anti-drug antibody specific for the LNA part of an LNA-containing drug.

### Application of Anti-LNA Antibodies According to the Invention in Pharmacokinetic (PK) Assays

For the detection of LNA-containing drug molecules, LNA-specific antibodies according to the current invention can be used as capture and/or detection reagents in an immunoassay setup. The antibodies according to the current case invention are LNA-specific antibodies but are not specific for a defined sequence of the LNA oligonucleotide. Thereby it is now possible to use the LNA-specific antibodies according to the current invention as a generic assay reagent in immunoassays, e.g., to detect specifically LNA-containing drug molecules, but independent of the sequence of the LNA in the LNA-containing drug molecules.

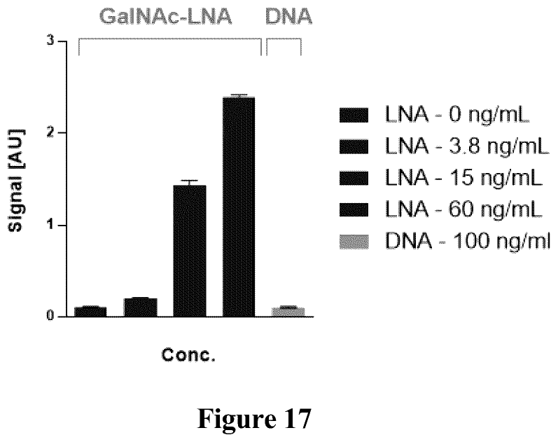

In an exemplary immunoassay, the LNA-specific monoclonal antibody according to the current invention produced by clone 1.9.21 as exemplary capture antibody in combination with a GalNac-LNA-specific detection antibody was used for the quantitative detection of a GalNAc-conjugated LNA drug (see FIG. 17).

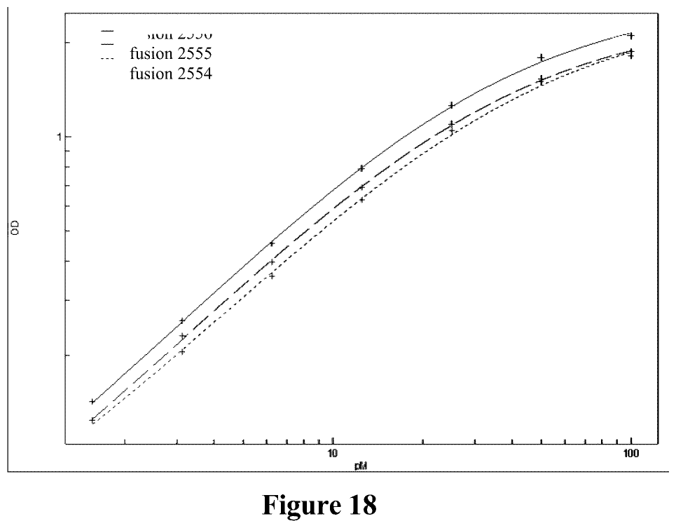

In another example, LNA-specific monoclonal antibody produced by clone 1.9.21 was used as capture antibody in combination with a human IgG specific detection antibody for the quantitative detection of antibody-ASO conjugate drug. The quantification of the antibody-antisense oligonucleotide (ASO) conjugates was carried out using a sandwich enzyme-linked immunosorbent assay (ELISA). Antibody-ASO conjugate containing standards and diluted plasma samples were prepared at double concentration (2×) in a pre-dilution plate. The first detection antibody conjugated to digoxygenin (DIG) as detectable label that is targeting the Fc-region of human IgG was also prepared at a 2× concentration in a separate pre-dilution plate. Complex formation was initiated by combining the calibrator/sample pre-dilution solution with the detection antibody solution, followed by incubation. A biotin-labeled LNA-specific antibody according to the current invention was used as capture antibody after immobilization on a streptavidin-coated microtiter plate (SA-MTP). The complex formation solution was transferred to the plate with the immobilized capture antibody and incubated. Thereafter, the second detection antibody, an anti-digoxygenin antibody conjugated to a peroxidase (POD), was added and incubated. Visualization of the immobilized complexes was achieved by adding BM Blue (TMB) solution and the optical density (OD) was measured at 680 nm (reference wavelength 450 nm) until a maximum OD of 0.7 was reached. The reaction was terminated by adding of H2SO4, causing the reaction product to turn yellow. Endpoint measurement was performed at 450 nm (reference 690 nm), with the highest standard reaching a maximum OD of 2.2. The quantification of the antibody-ASO conjugate was performed by back-calculating the OD values using a non-linear 4-parameter Rodbard-205 curve fitting function, with the standard calibration curve prepared in hybridization buffer. Example of calibration curves of three different antibody-ASO conjugates are shown in FIG. 18 and Table 5-1.

TABLE 5-1
Standard curves obtained with three different antibody-ASO conjugates using an antibody
according to the current invention as capture antibody (IgG1 Fc-region with anti-TfR Fab
1026 fused to the N-terminus of one Fc-region polypeptide with ASO 307 (SEQ ID NO: 132)
conjugated to position 446 (fusion 2556) or position 341 (fusion 2555) or position 297
(fusion 2554) (numbering according to Kabat) via a linker using transglutaminase).

| concentration [pM] | 100 | 50 | 25 | 12.5 | 6.3 | 3.1 | 1.6 | 0 |
|---|---|---|---|---|---|---|---|---|
| mean signal | 2.1010 | 1.7895 | 1.2595 | 0.7910 | 0.4570 | 0.2585 | 0.1425 | 0.0215 |
| fusion 2556 | | | | | | | | |
| [AU] | | | | | | | | |
| mean signal | 1.8685 | 1.5325 | 1.0955 | 0.6880 | 0.3980 | 0.2310 | 0.1245 | 0.0210 |
| fusion 2555 | | | | | | | | |
| [AU] | | | | | | | | |
| mean signal | 1.8140 | 1.5005 | 1.0445 | 0.6280 | 0.3590 | 0.2060 | 0.1245 | 0.0280 |
| fusion 2554 | | | | | | | | |
| AU] | | | | | | | | |

Thus, one aspect of the current invention is an immunoassay for the determination of the presence or amount of an LNA-containing drug in a sample, wherein an antibody according to the current invention is used as capture antibody or as detection antibody.

In one embodiment the immunoassay is a bridging ELISA. In one embodiment the bridging ELISA comprises the steps of
a) immobilizing an LNA-specific antibody according to the current invention on a solid surface,
b) incubating the solid surface-immobilized LNA-specific antibody obtained in step a) with a sample suspected to contain the LNA-containing drug and thereby obtaining an immobilized LNA-specific antibody-drug complex,
c) determining the presence or amount of the immobilized LNA-specific antibody-drug complex by incubating the surface obtained in step b) with a detection antibody conjugated to a detectable label and correlating the signal of the detectable label to the calibration curve and thereby determining the presence or amount of the LNA-containing drug.

In one embodiment the detection antibody is an antibody specifically binding to the non-LNA part of the LNA-containing drug. In one embodiment the LNA-containing drug is an antibody-LNA conjugate. In one embodiment the detection antibody specifically binds to the Fc-region of the antibody-LNA conjugate.

In one embodiment the immunoassay is a bridging ELISA. In one embodiment the bridging ELISA comprises the steps of
a) immobilizing a capture antibody specifically binding to the non-LNA part of the LNA-containing drug on a solid surface,
b) incubating the solid surface-immobilized capture antibody obtained in step a) with a sample suspected to contain the LNA-containing drug and thereby obtaining an immobilized capture antibody-drug complex,
c) determining the presence or amount of the capture antibody-drug complex by incubating the surface obtained in step b) with an LNA-specific antibody according to the current invention conjugated to a detectable label and correlating the signal of the detectable label to the calibration curve and thereby determining the presence or amount of the LNA-containing drug.

In one embodiment the capture antibody is an antibody specifically binding to the non-LNA part of the LNA-containing drug. In one embodiment the LNA-containing drug is an antibody-LNA conjugate. In one embodiment the capture antibody specifically binds to the Fc-region of the antibody-LNA conjugate.

Use of an antibody according to the current invention as a capture or detection antibody in an immunoassay.

In one embodiment the immunoassay is a bridging ELISA or a direct ELISA.

In one embodiment the immunoassay is for the determination of the presence or amount of an LNA-containing drug. In one embodiment the LNA-containing drug is an antibody-LNA conjugate.

### X-Ray Structure of LNA-Binding Antibodies According to the Current Invention

Crystallization, Data Collection and Structure Determination of Anti-LNA Antibody Produced by Clone 1.9.21 in Fab Format (Fab 0699) in Complex with LNA tCac (ASO 980)

Fab 0699 was concentrated to 24.6 mg/ml. Crystal screening was performed at 21° C. in sitting drop vapor diffusion experiments using a drop sizes of 200 nL with 50% and 70% (w/v) amount of protein. Plate shaped crystals appeared within eight days. The complex with LNA was obtained by soaking crystals for 16 hours in a solution of 2 mM of ASO 980 (SEQ ID NO: 133).

For data collection crystals were flash cooled at 100 K in crystallization solution supplemented with 20% ethylene glycol and X-ray diffraction data were collected. The collected data has been processed, scaled and analyzed for anisotropy.

The crystals of the complex belong to space group C2 with cell axes of a=126.61 Å, b=59.60 Å, c=62.50 Å with P=94.30° and diffract to a resolution of 1.57 Å. The structure was determined by molecular replacement using the coordinates of an in house Fab as search model. Difference electron density guided the exchange of amino acids according to the sequence differences to the search model and for model building of the LNA. The structure was refined and manually rebuilt. Data collection and refinement statistics are summarized in Table 6-1. All graphical presentations were prepared with PYMOL (The Pymol Molecular Graphics System, Version 1.7.4. Schrödinger, LLC.)

TABLE 6-1
Data collection and refinement statistics for
fab 0699 in complex with ASO 980.

| Data collection | |
|---|---|
| Space group | C2 |
| Cell dimensions | |
| a, b, c (Å) | 126.61, 59.60, 62.50 |
| α, β, γ (°) | 90, 94.30, 90 |
| Resolution (Å) | 1.57 |
| Rₘₑₐₛ | 0.043 (0.634) |
| I/σI | 10.44 (1.94) |
| CC(1/2) | 0.996 (0.579) |
| Completeness (%) | 97.2 (84.2) |
| Redundancy | 3.19 (1.94) |
| Refinement | |
| Resolution (Å) | 53.90-1.57 |
| No. reflections | 63072 |
| R_{work}/R_{free} | 18.90/21.70 |
| No. atoms | |
| Protein | 3226 |
| ASO 980 | 79 |
| Water | 538 |
| B-factors | |
| Protein | 31.34 |
| ASO 980 | 40.96 |
| Water | 45.44 |
| R.m.s. deviations | |
| Bond lengths (Å) | 0.010 |
| Bond angles (°) | 1.09 |

*Values in parentheses are for highest-resolution shell.

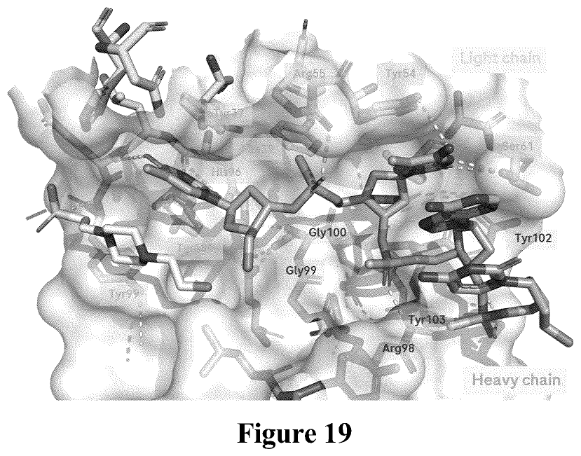

Structure of anti-LNA antibody produced by clone 1.9.21 in Fab format (fab 0699) in complex with LNA tCac (ASO 980) The crystal structure of the complex of fab 0699 with LNA ASO 980 was determined at a resolution of 1.57 Å (FIG. 19). The binding site of the LNA locates in the groove between heavy and light chain with major contributions to the paratope by the light chain with all three CDRs involved whereas the heavy chain mainly binds through CDR3 (see Table 6-2). Three main interaction motifs of the LNA with the Fab can be observed. This includes polar interactions of the base at position 1 with Fab, a hydrophobic pocket which accommodates the modified LNA sugar in position 2 and a pi-pi stacking ensemble formed by bases in position 2-4 in combination with side chain of Tyr54L. In more detail, the thymine base in position 1 of the LNA extends towards HVR-L2 and HVR-L3 and hydrogen bonds with the main chain carbonyl atom and side chain of His96L. Additional pi-pi stacking interactions are picked up with sidechain of Tyr37L. The sulfur atom of the phosphorothioate points away from the solvent towards side chain of Phe39L. In position 2 the modified LNA sugar locates into a pocket formed by the CDR3 of the heavy chain with additional contributions from side chains of Phe39L, Val51L and Tyr54L. The sulfur of the phosphorothioate in position 2 faces the solvent whereas the oxygen of the phosphate group entertains a hydrogen bond to side chain of Arg98H. Bases in position 2-4 form together with side chain of Tyr54L a pi-pi stacking ensemble with the bases facing towards the solvent region. This may explain the tolerability of the Fab against different base types in these positions.

TABLE 6-2
Interfacing residues of Fab 0699 with ASO 980.

| Paratope residues | |
|---|---|
| ASO 980 with light chain | ASO 980 with heavy chain |
| His31 | Tyr32 |
| Asn33 | Arg98 |
| Tyr37 | Gly99 |
| Phe39 | Gly100 |
| Val51 | Gly 101 |
| Tyr54 | Tyr102 |
| Arg55 | Tyr103 |
| Ala60 | |
| Ser61 | |
| His96 | |
| Leu97 | |
| Tyr101 | |

One aspect according to current invention is an antibody specifically binding to an LNA-modified nucleic acid, characterized in that the antibody has a polar interaction with the base at position 1 of the LNA-modified nucleic acid, a hydrophobic pocket which accommodates the modified LNA sugar in position 2, and a pi-pi stacking ensemble formed by bases in position 2-4 in combination with side chain of Tyr at position 54 according to Kabat numbering in the light chain variable domain.

In one embodiment of the antibody, the thymine base in position 1 of the LNA extends towards HVR-L2 and HVR-L3 and hydrogen bonds with the main chain carbonyl atom and side chain of a His at position 96 according to Kabat numbering on the light chain variable domain.

In one embodiment of the antibody, additional pi-pi stacking interactions are picked up with sidechain of a Tyr at position 37 according to Kabat numbering in the light chain variable domain.

In one embodiment of the antibody, the sulfur atom of the phosphorothioate points away from the solvent towards the side chain of a Phe at position 39 according to Kabat numbering in the light chain variable domain.

In one embodiment of the antibody, in position 2 the modified LNA sugar locates into a pocket formed by the HVR-H3 with additional contributions from side chains of a Phe at position 39, a Val at position 51 and a Tyr at position 54 of the light chain variable domain (all positions according to Kabat numbering).

In one embodiment of the antibody, the sulfur of the phosphorothioate in position 2 faces the solvent whereas the oxygen of the phosphate group entertains a hydrogen bond to the side-chain of an Arg at position 98 according to Kabat numbering of the heavy chain.

In one embodiment of the antibody, the bases in position 2-4 form together with the side chain of a Tyr at position 54 according to Kabat numbering of the light chain variable domain a pi-pi stacking ensemble with the bases facing towards the solvent region.

Combining LNA-Binding Sites with Anti-TfR-ASO Conjugates To generate antibodies with an LNA-modified ASO as payload, Kutzneria albida Transglutaminase (KTG)-mediated site-directed conjugation combined with a click reaction to attach the LNA-modified ASO the antibody was applied (see, e.g., WO 2023/118398).

Briefly, antibody tagged with the amino acid sequence YRYRQ (Q-tag; SEQ ID NO: 134) was first enzymatically linked to an azide-containing linker tagged with an amino acid sequence RYESK (K-tag; SEQ ID NO: 136) using the KTG enzyme. Desired products were separated from KTG enzyme and unreacted linkers by size exclusion chromatography. In the second step, an LNA-modified ASO conjugated to a BCN group was attached to the antibody-azide linker through a click reaction. Desired products were separated from unreacted ASO using size exclusion chromatography. Conjugation efficiency and the molecular composition were confirmed by mass spectrometry.

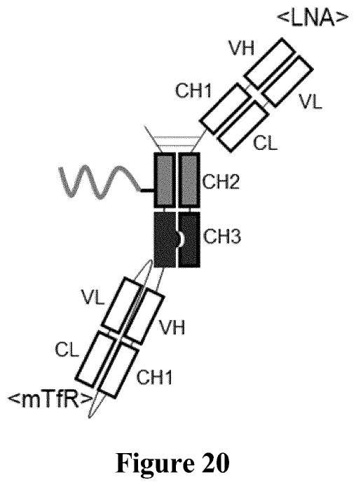

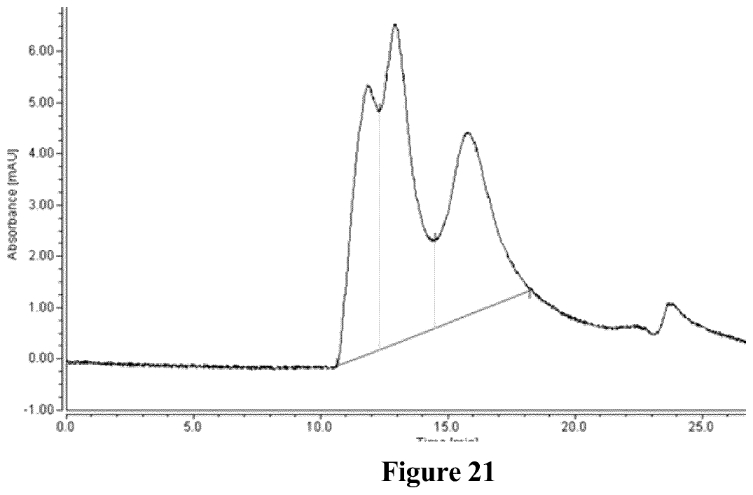

Attachment of the LNA-modified ASO to a bispecific anti-TfR/LNA antibody (structure sketch shown in FIG. 20) resulted in the formation of dimers or multimers, which appear as separate peaks before the fraction that contains the monomeric antibody on an analytic size chromatography (SEC-HPLC; FIG. 21).

Native mass spec analysis (SEC-MS) confirmed those peaks contain dimers. With mass spectrometric analysis under denaturing condition (RP-MS) a conjugation efficiency with an average DAR of 0.7-0.8 was determined (Table 7-1). These results suggest that conjugation of an LNA-modified ASO to an anti-LNA antibody allows the formation of dimers and interferes with the ASO conjugation.

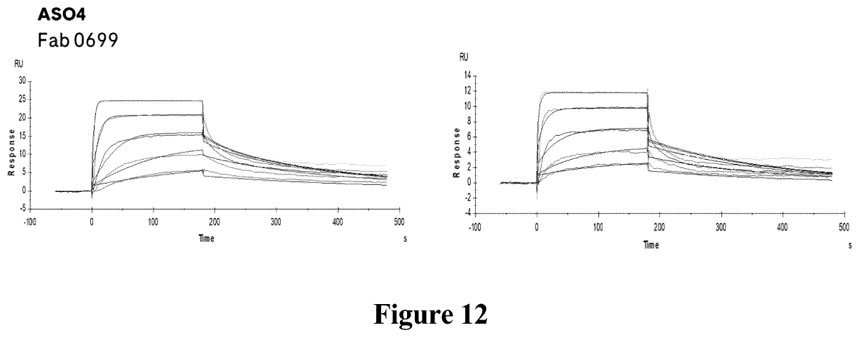

TABLE 7-1
Mass spectrometric results for the bispecific anti-TfR/LNA
antibody conjugated to an LNA-modified ASO.

| | DAR | identity | integrity | |
|---|---|---|---|---|
| antibody | (RP-MS) | (RP-MS) | RP-MS | SEC-MS |
| bispecific anti- | 0.7 | confirmed | antibody | dimers |
| mTfR/LNA | | | with linker | detected |
| antibody (fab 8D3 | | | only | |
| and fab 0699) with | | | detected | |
| LNA-modified | | | | |
| ASO 576 (SEQ ID | | | | |
| NO: 135) | | | | |
| conjugated at | | | | |
| position 297 of the | | | | |
| knob heavy chain | | | | |
| (inserted tag of | | | | |
| SEQ ID NO: 143; | | | | |
| Kabat EU index); | | | | |
| see FIG. 12; | | | | |
| bsAb 4318 | | | | |
| bispecific anti- | 0.8 | Confirmed | Antibody | Dimers |
| huTfR/LNA | | | with linker | detected |
| antibody (fab 1026 | | | only | |
| and fab 0699) with | | | detected | |
| LNA-modified | | | | |
| ASO 576 (SEQ ID | | | | |
| NO: 135) | | | | |
| conjugated at | | | | |
| position 297 of the | | | | |
| knob heavy chain | | | | |
| (inserted tag of | | | | |
| SEQ ID NO: 143; | | | | |
| Kabat EU index); | | | | |
| see FIG. 12; | | | | |
| bsAb 4319 | | | | |

To evaluate whether the format impacts the conjugation yield and/or the dimerization, different bispecific antibodies were generated and tested (see, e.g., Dengl, S., et al., Nat. Commun. 11 (2020) 4974; WO 2019/077092).

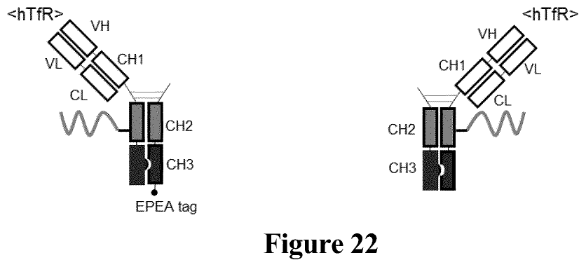

Additionally, the LNA-modified ASO was conjugated to a human transferrin receptor binding monovalent antibody, i.e. without a LNA-binding site. Mass spec analysis of these samples under denaturing condition (RP-MS) confirmed the successful conjugation with an average DAR close to 1 (see FIG. 22; Table 7-2).

TABLE 7-2
Mass spectrometric results for the monospecific anti-TfR
antibody conjugated to an LNA-modified ASO.

| | DAR | identity | integrity |
|---|---|---|---|
| antibody | (RP-MS) | (RP-MS) | (RP-MS) |
| monospecific anti- | 0.9 | Confirmed | >90% main |
| hTfR antibody (fab | | | product |
| 1026) conjugated at | | | |
| position 297 of the | | | |
| knob heavy chain to | | | |
| the LNA modified | | | |
| ASO 576 (inserted | | | |
| tag of SEQ ID NO: | | | |
| 143; numbering | | | |
| according to Kabat | | | |
| EU index) using | | | |
| FORCE and KTG | | | |
| transglutaminase; | | | |
| mAb 5650 | | | |
| monospecific anti- | 1.1 | Confirmed | >90% main |
| hTfR antibody (fab | | | product |
| 1026) conjugated at | | | |
| position 297 of the | | | |
| hole heavy chain to | | | |
| the LNA modified | | | |
| ASO 576 (inserted | | | |
| tag of SEQ ID NO: | | | |
| 143; numbering | | | |
| according to Kabat | | | |
| EU index) using | | | |
| recombinant | | | |
| expression and KTG | | | |
| transglutaminase; | | | |
| mAb 3732 | | | |

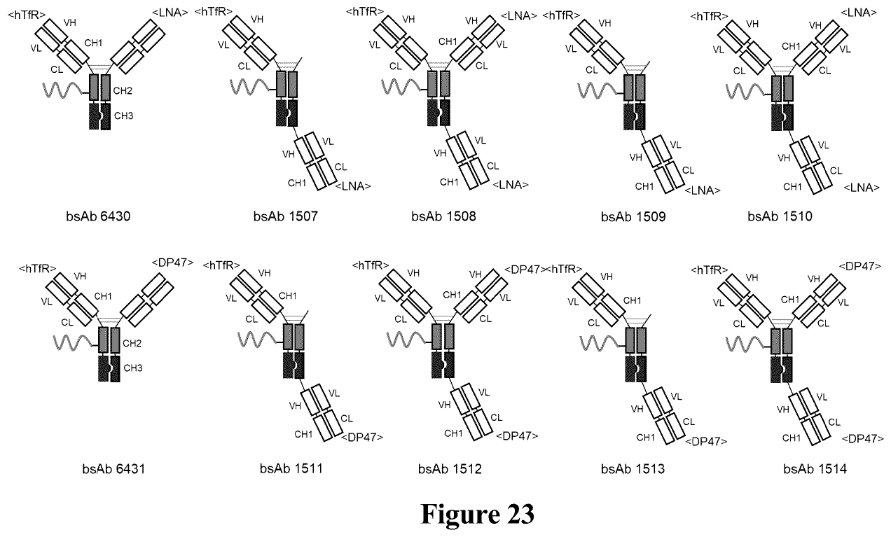

Different bispecific formats with a binding site specifically binding to an LNA-modified ASO and a second binding site with germline sequence (DP47) and, thus, not binding to a target as shown in FIG. 23 have been produced.

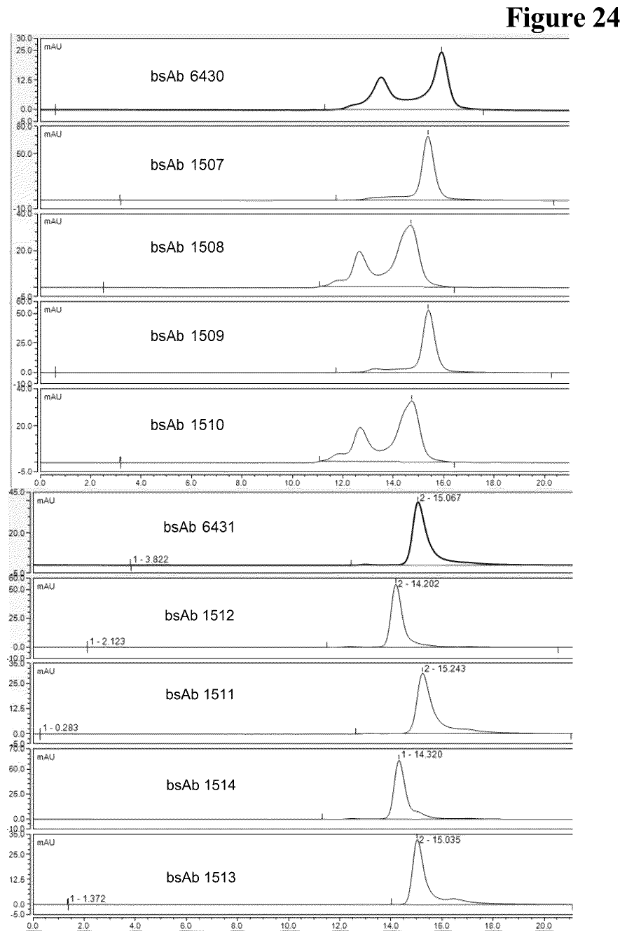

Size Exclusion Chromatography showed that the N-terminally linked anti-LNA binding site resulted in the formation of high molecular weight products. Additionally it has been found that a single C-terminally linked anti-LNA binding site conjugated with a flexible peptidic linker to the C-terminus of the Fc-region (e.g., a 20 amino acid or 40 amino acid GS-linker) resulted in the formation of products with more than 90% of monomer. Products with the germline binding site (DP47), i.e. non-binders instead of the anti-LNA binding site, regardless of the format, resulted in the formation of products with more than 90% of monomer, confirming that the presence of an N-terminally linked anti-LNA binding site results in aggregate formation. The respective SEC chromatograms are shown in FIG. 24.

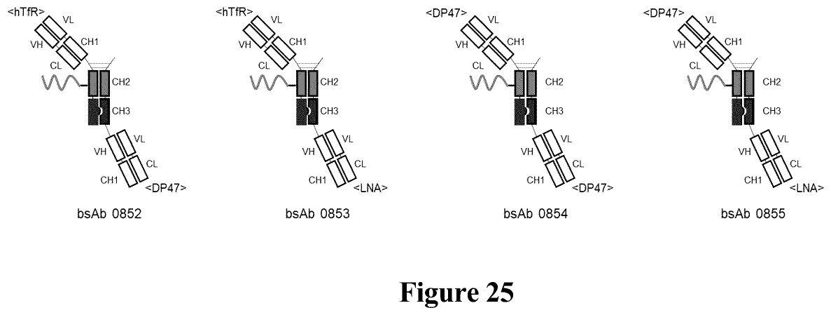

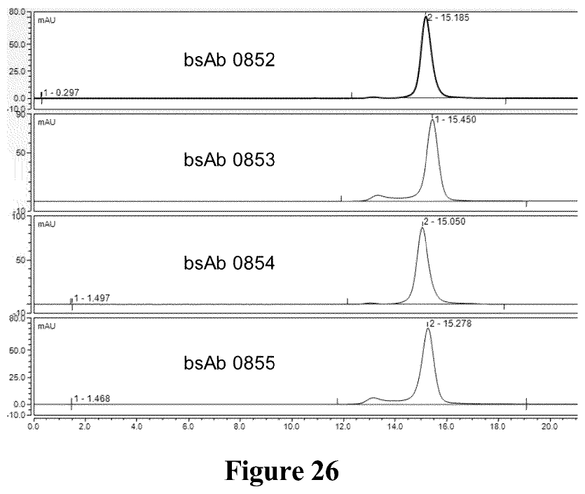

Based on these results, bispecific antibodies conjugated to an LNA-modified ASO were also generated by enzymatic conjugation, i.e. KTG-mediated site-directed conjugation combined with click reaction (FIG. 25). The resulting molecules formed predominantly monomers (FIG. 26).

Mass spectrometric analysis showed a good conjugation efficiency, confirming the good biophysical behavior of the identified format (Table 7-3).

TABLE 7-3
Mass spectrometric results of the bispecific anti-TfR/LNA
antibodies conjugated to an LNA-modified ASO 576 with
20 amino acid peptidic GS linker as produced with enzymatic
conjugation followed by click chemistry conjugation.

| | DAR | Identity | Integrity |
|---|---|---|---|
| antibody | (RP-MS) | (RP-MS) | (RP-MS) |
| bsAb 0852 | 0.85 | confirmed | >90% main product |
| bsAb 0853 | 0.95 | confirmed | >90% main product |
| bsAb 0854 | 0.89 | confirmed | >85% main product |
| bsAb 0855 | 1 | confirmed | >85% main product |

One aspect according to the current invention is a bispecific antibody comprising an Fc-region and a first binding site specifically binding to an LNA-modified ASO that is conjugated to the Fc-region and a second binding site not binding to an LNA-modified ASO that is also conjugated to the Fc-region, wherein the binding site specifically binding to the LNA-modified ASO is conjugated to the C-terminus of the Fc-region of the bispecific antibody.

In one embodiment, the bispecific antibody comprises no binding site/is free of binding sites specifically binding to an LNA-modified ASO conjugated to the N-terminus of the Fc-region of the bispecific antibody.

In one preferred embodiment, the bispecific antibody comprises exactly one binding site specifically binding to an LNA-modified ASO.

In one embodiment the binding site specifically binding to an LNA-modified ASO is an antibody fragment. In one embodiment, the antibody fragment is selected from the group of antibody fragments comprising a Fab, a scFab, a scFv, a dual binding Fab and a DutaFab. In one preferred embodiments the antibody fragment is a Fab or a scFab.

In one embodiment, the peptidic linker comprises between and including 15 and 50 amino acid residues. In one embodiment, the peptidic linker comprises between and including 20 to 40 amino acid residues. In one preferred embodiment, the peptidic linker comprises about 20 amino acid residues and is solely made of glycine and serine residues.

In one preferred embodiment, the binding site specifically binding to the LNA-modified ASO comprises the HVRs of the anti-LNA antibody produced by clone 1.9.21.

It has further been found that the LNA binding site in a bispecific anti-TfR/LNA antibody improves the targeted uptake or recycling of the LNA-modified ASO conjugated to the antibody.

### Covalent and Non-Covalent Conjugate Comprising an LNA-Modified ASO Complexed by the LNA-Antibody According to the Invention Improves Biological Activity of the ASO

To show further beneficial properties of the anti-LNA antibodies according to the current invention in cellular assays, a colocalization assay was done using the human blood-brain-barrier endothelial cell line hCMEC/D3.

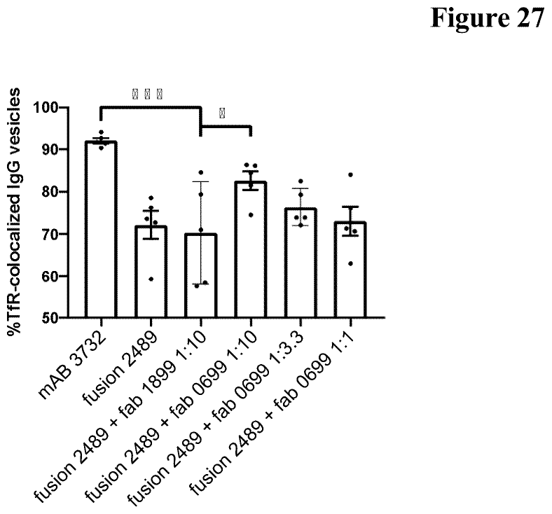

In more detail, 1) an unconjugated anti-TfR antibody, 2) an anti-TfR antibody conjugated to an LNA-modified ASO, 3) a non-covalent complex of an anti-LNA antibody Fab according to the current invention produced by clone 1.9.21 with an anti-TfR antibody conjugated to an LNA-modified ASO and 4) a mixture of a non-binding Fab (DP47 Fab) and an anti-TfR antibody conjugated to an LNA-modified ASO were applied to hCMEC/D3 cells in the culture medium for 3 hours. In the last 20 min of incubation, fluorophore-labelled transferrin was added to label the transferrin receptor. The cells were fixed and permeabilized and the IgGs were labeled by an anti-human IgG antibody. Images were acquired and an object-based colocalization analysis between IgG and transferrin receptor was carried out. Results are shown in FIG. 27.

It can be seen that conjugation of the LNA-modified ASO to the anti-TfR antibody reduced IgG colocalization with the transferrin receptor, indicating the LNA-modified ASO payload contributes to the transferrin receptor-independent uptake. However, pre-incubate the anti-TfR antibody conjugated to the LNA-modified ASO with the anti-LNA Fab, but not with the non-binding fab DP47 rescued the IgG colocalization with the transferrin receptor in a dose-dependent manner, indicating the addition of an anti-LNA antibody according to the current invention reduces the transferrin receptor-independent uptake.

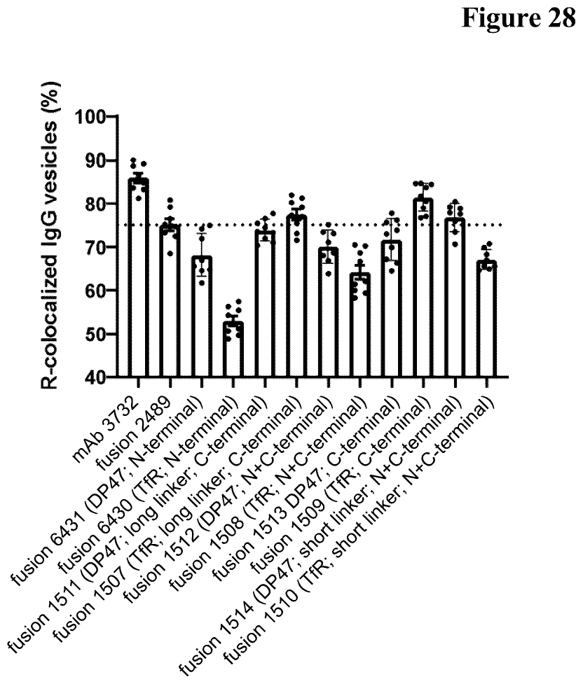

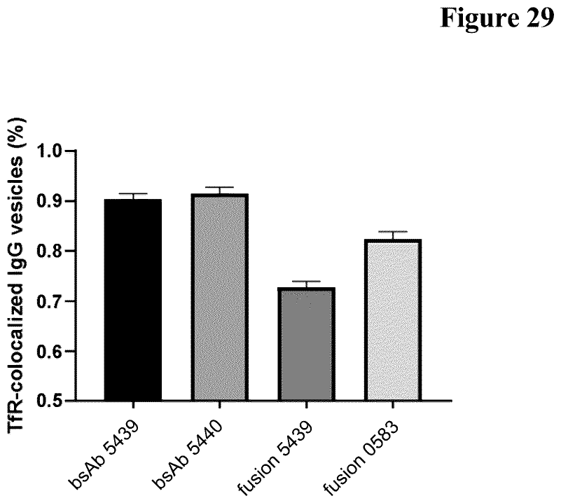

To further access functionality of the anti-LNA antibody produced by clone 1.9.21 as an example of the antibodies according to the current invention, FORCE method (see, e.g. Dengl, S., et al., Nat. Commun. 2020 (11) 4974) and site-directed enzymatic (KTG) followed by azide-BCN click conjugation were used to generate a covalently-linked intramolecular binder, a bispecific anti-TfR/LNA antibody conjugated to an LNA-modified ASO payload as well as the corresponding control (anti-DP47/LNA antibody; fab DP47 and fab 0699). Conjugates generated by the FORCE method and the conventional recombinant expression method were tested in the colocalization assay as described above. Results are shown in FIG. 28 and FIG. 29, respectively.

The assay shows that C-terminal conjugation of an LNA binding site improves colocalization with the transferrin receptor as compared to the corresponding control molecules with a non-binding site. This confirms that a C-terminally linked LNA binding site improves the transferrin receptor-mediated uptake or/and recycling. Of note, the same molecules also formed predominantly monomers in HPLC-SEC.

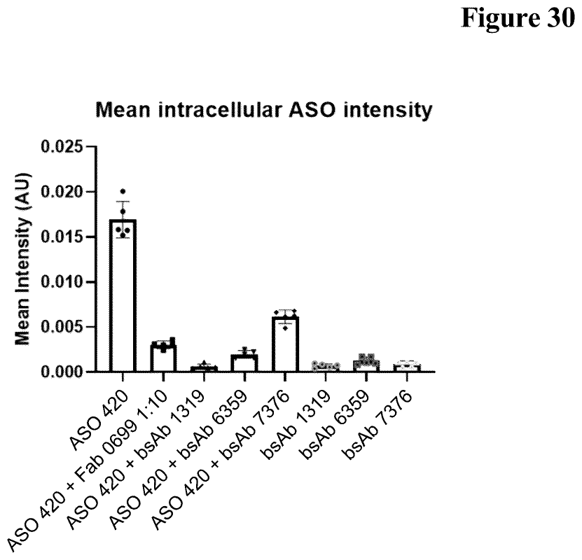

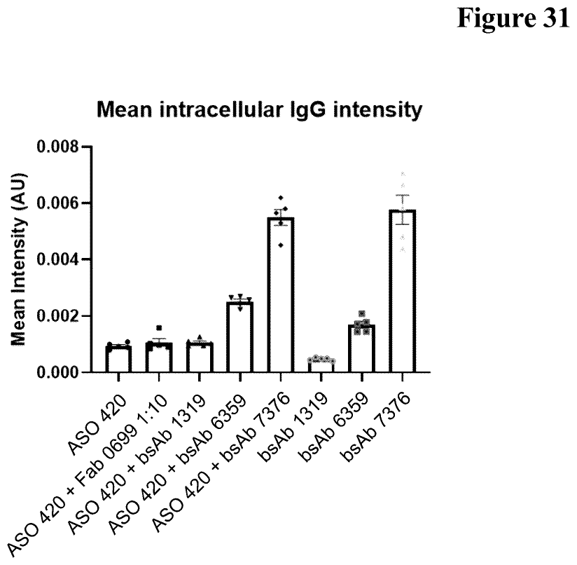

To determine if the LNA binding site may mediate the uptake of the LNA-modified ASO in a non-covalent manner, the format with two C-terminally linked LNA binding sites was used. It has been found by SPR that this format can drive 1:1 antibody-to-ASO ratio of binding with high avidity (kD around 3 μM) (see above). An anti-LNA antibody derived from the antibody produced by clone 1.9.21 were premixed with an LNA-modified ASO (Atto647N-linked ASO) at 1:1 molar ratio and applied to hCMEC/D3 cells at 37° C. for 3 hr. In the last 20 min of the incubation, fluorophore-labelled transferrin (Alexa555-transferrin) was added to label the transferrin receptor. Cells were fixed and immunostained. Cell nuclei and plasma membranes were stained by DAPI and CellMask (ThermoFischer #H32720), respectively. Intracellular (defined by CellMask staining) LNA-modified ASO and IgG intensities were quantified (FIGS. 30 and 31).

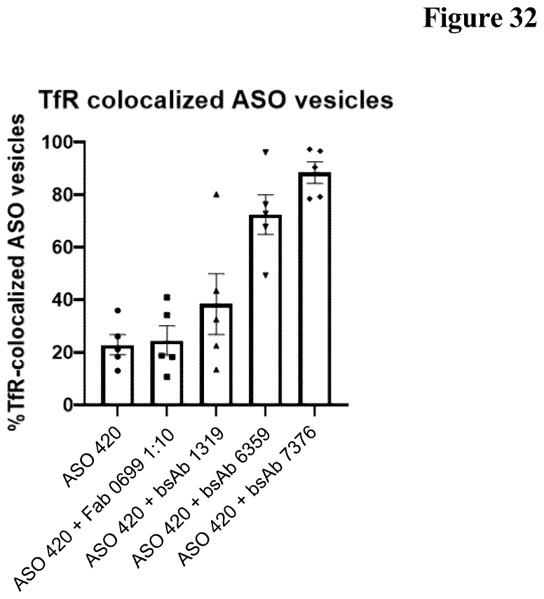

The results show that the two C-terminally linked LNA binding sites can hold the fluorophore-labelled LNA-modified ASO, and depending on the N-terminal binder the antibody can either prevent (non-binding antibody (Nada); the CDRs were intentionally replaced by other amino acids to destroy the binding with the target but the net charges etc. were kept similar) or drive the uptake (hTfR low, hTfR high) of the LNA-modified ASO. Object-based colocalization analysis revealed that premixing Atto647N-ASO with antibodies with a hTfR binder significantly increased the colocalization of Atto647N-ASO with transferrin receptor (FIG. 32), further indicating that the uptake of ASO is driven by the antibody.

To assess the transcytosis efficiency of the antibodies and conjugates according to the current invention, a 3D BBB (Blood-Brain Barrier) spheroid assay was conducted (see, e.g., Simonneau et al., Fluids Barriers CNS (2021), Kassianidou and Simonneau et al., Bio. Protoc. (2022)). In more detail, primary human astrocytes, human brain microvascular pericytes, and human cerebral microvascular endothelial cells were maintained separately in the respective culture media. Spheroids were generated by re-suspending the cells in a 1:1:1 ratio and grown to allow self-assembly of the multicellular spheroids. After 48 h of assembly, BBB spheroid arrays were incubated with the tested molecules in media for 4 h at 37° C. After incubation, BBB spheroids were washed and fixed in PFA. Samples were permeabilized and stained with a fluorescently labelled anti-human FcY (H+L) antibody. Finally, the samples were transferred to cover glasses and imaged for quantitative analysis using a Leica Microsystems, Thunder Imager 3D Assay. The Instant Computational Clearing (ICC) algorithm by Leica was applied to the images. Quantitative analysis was performed using a custom-made automated Fiji script that segments individual spheroids and measures the mean fluorescence intensity projection within 75% of spheroid area. Briefly, the multi-channel z-stack was converted into a multi-channel maximum projection image. The macro then splits the multi-channel maximum projection image into individual channel images, and takes the DAPI maximum projection image to create a mask via thresholding. The macro then converts the mask into a region of interest (ROI) based on its size and shape. The ROIs are then reduced to 75%, to cover only the core of the spheroid, and exclude measurements from the endothelial surface of the spheroid. The shrunk ROIs are overlaid on top of the channel of interest, and relevant measurements are calculated. Fluorescence intensity is reported per μm², by dividing raw integrated density over area (μm²).

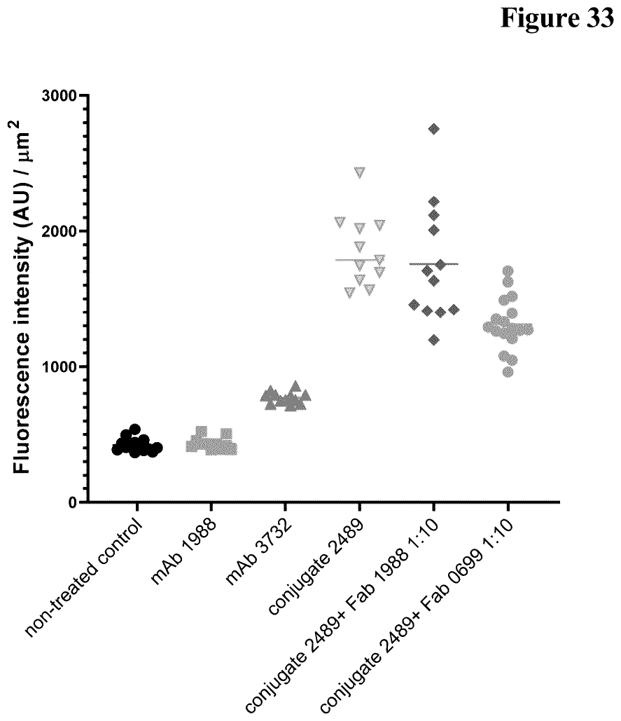

In the first set of experiments (FIG. 33), the anti-hTfR antibody (fab 1026) conjugated to the LNA-modified ASO largely increased the fluorescence intensity within the spheroid core, suggesting that the LNA-modified ASO payload caused non-specific uptake and transcytosis of conjugates into the spheroids. However, pre-incubating of the anti-hTfR antibody conjugated to an LNA-modified ASO with excess of anti-LNA antibody Fab fragment of the anti-LNA antibody produced by clone 1.9.21, but not with an excess of non-binding antibody Fab (fab DP47), decreased the fluorescence intensity within the spheroid core. This result shows that the LNA-binder can reduce large aggregates caused by the LNA-modified ASO conjugated to the anti-hTfR antibody.

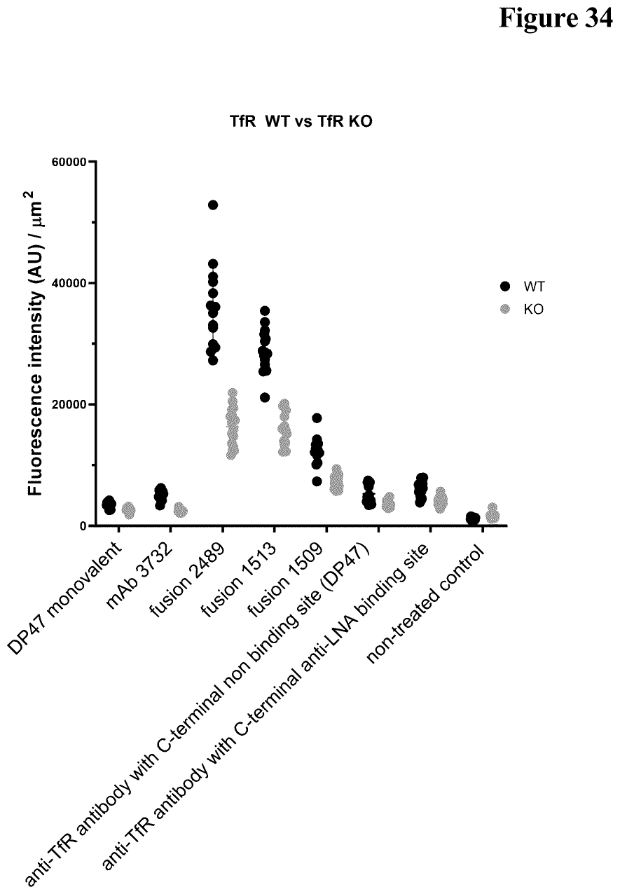

In a further set of experiments (FIG. 34), anti-hTfR antibodies (fab 1026) conjugated to an LNA-modified ASO generated by the FORCE technology were tested in spheroids assembled with wild-type or human TfR knock-out brain microvascular endothelial cells. The human TfR knock-out spheroids were used to access transferrin receptor independent transcytosis. The format with a C-terminally linked LNA binding site was selected, as this format predominantly forms monomers in the analytical size exclusion column. The construct with a covalently-linked LNA binding site, but not with non-binding site (fab DP47), significantly reduced fluorescence intensity within both the wild-type and human transferrin receptor knock-out spheroids. Therefore, a C-terminally linked LNA binding site can reduce the transferrin receptor independent uptake and transcytosis of the anti-hTfR antibody conjugated to an LNA-modified ASO.

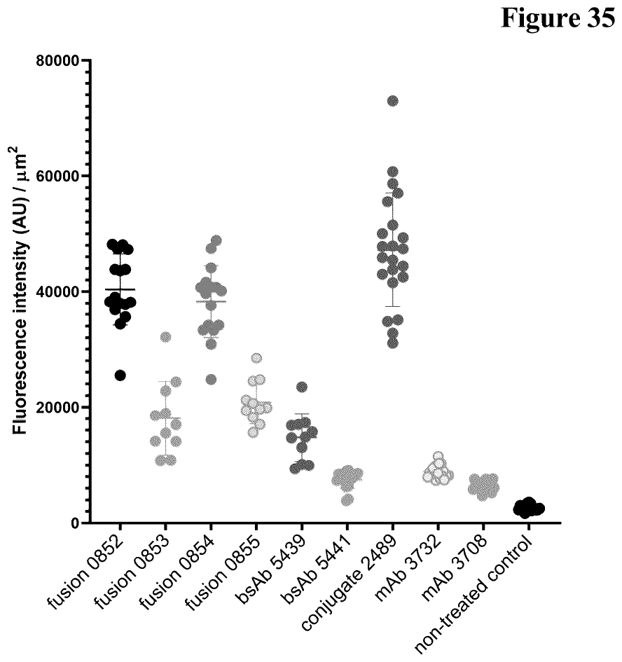

In a further set of experiments (FIG. 35), anti-hTfR antibodies (fab 1026) conjugated to an LNA-modified ASO generated by recombinant expression were tested in BBB spheroids. The results confirmed that a C-terminally conjugated LNA binding site can reduce non-specific uptake and transcytosis driven by the LNA-modified ASO payload.

To access the non-specific cellular accumulation of antibody-ASO conjugates, the Large molecule Unspecific Clearance Assay (LUCA) was used (see WO 2021/204743). The LUCA assay uses primary human liver sinusoidal endothelial cells. Data is acquired by labeling the antigen binding molecules with a pH-sensitive dye exhibiting high fluorescence, when accumulating in the late endosome and lysosome (acidic pH 5.5) and low fluorescence when remaining outside the cell (neutral pH 7.4). Human or animal endothelial cells are incubated with labeled antibodies for 2 and 4 hours and the fluorescent readout is recorded using a flow cytometer. The geo-mean intensities are used for linear regression analysis after subtraction of background signal (cellular autofluorescence) and normalization to the fluorescence of the dosing solution (to account for differences in dye-to-antibody ratio). The extracted slopes form, when normalized to standard antibodies, the so-called relative LUCA rate.

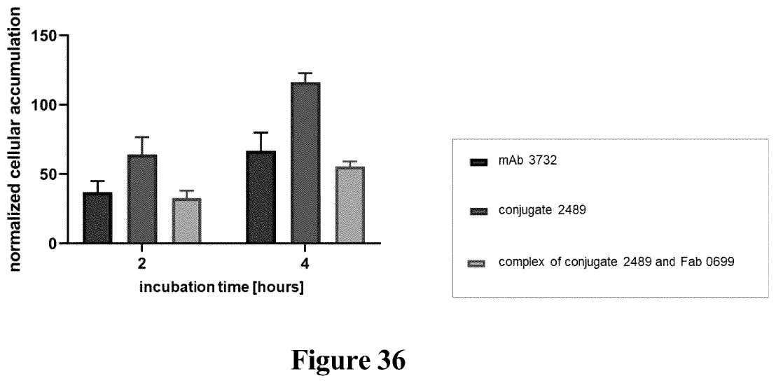

In the first set of experiments (FIG. 36), anti-hTfR antibody (fab 1026), anti-hTfR antibody (fab 1026) conjugated to an LNA-modified ASO, and non-covalent complex of anti-hTfR-antibody (fab 1026) conjugated to LNA-modified ASO with anti-LNA fab 0699 (1:10 ratio for incubation) were tested in the LUCA setting. At both time points (2 h and 4 h) the presence of the ASO-payload increased the normalized cellular accumulation, contributed by non-specific and target mediated uptake. However, pre-incubation with an excess of anti-LNA Fab decreased the cellular accumulation of the anti-hTfR antibody conjugated to an LNA-modified antibody.

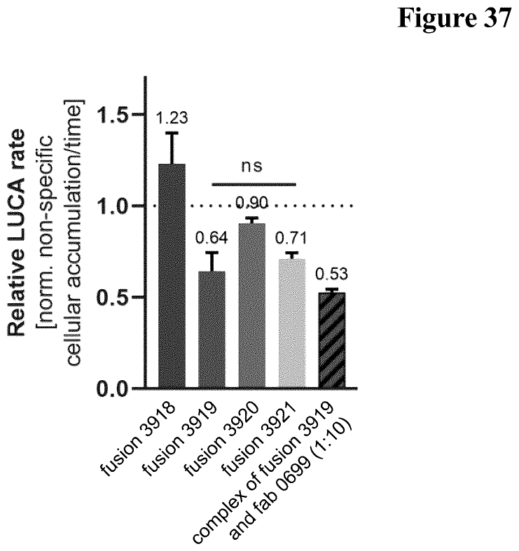

In a further set of experiments (FIG. 37), ASO conjugated to antibodies with an N-terminal <nada> non-binding site and a C-terminally linked non-binding site (fab DP47) or LNA binding site were tested in the LUCA setting. The relative LUCA rate (unspecific cellular accumulation) was decreased in the construct wherein the ASO was conjugated to an antibody with a C-terminally linked LNA binding site, as compared to ASO conjugated to a C-terminally linked non-binding site (fab DP47). Pre-incubating the anti-Nada/LNA antibody-ASO conjugate (<nada> and fab 0699) with 10× molar ratio of the anti-LNA fab 0699 did not further reduce the relative LUCA rate, suggesting that one C-terminally linked LNA binding site is sufficient to fully reduce the unspecific cellular accumulation caused by the ASO payload.

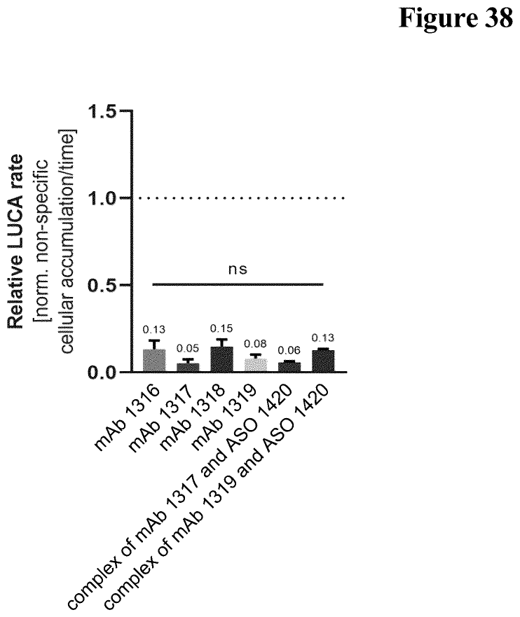

In a further set of experiments (FIG. 38), the antibody with a N-terminal <Nada> and two C-terminally linked LNA binding sites (fab 0699) with or without pre-incubation with equimolar amount of free ASO were tested in the LUCA setting. The two C-terminally linked LNA binding sites can hold one ASO with high avidity (confirmed by SPR). Addition of non-covalently linked ASO to the construct did not increase the relative LUCA rate, suggesting the ASO is protected by the two LNA binding sites and does not increase unspecific cellular accumulation.

### In Vivo Plasma Pharmacokinetics Study

The pharmacokinetics (PK) properties of antibody-ASO complexes and conjugates were assessed in an in vivo single-dose plasma PK study in male C57BL/6J mice. The animal grouping and dosing details are summarized in Table 8-1.

TABLE 8-1
Animal grouping and dose levels

| Group | Test Item | Dose [mg/kg] |
|---|---|---|
| 1 | ASO 827 | 0.93 |
| 2 | mAb 1927 | 40.9 |
| | ASO 827 | 0.93 |
| 3 | fusion 9545 | 21.3 |
| 4 | fusion 9544 | 21.5 |
| 5 | fusion 9543 | 21.5 |
| 6 | fusion 9542 | 21.6 |
| 7 | bsAb 1319 | 27.1 |
| | ASO 827 | 0.93 |
| 8 | bsAb 1926 | 27.1 |
| | ASO 827 | 0.93 |

Mice were administered intravenously (i.v.) with compounds (dose volume 5 mL/kg) listed in Table 8-1, including naked ASO (Group 1), ASO pre-incubated with a 3× molar ratio of IgG with one N-terminal anti-LNA antibody Fab fragment of the anti-LNA antibody produced by clone 1.9.21 (<LNA3> binding site; Group 2), ASO pre-incubated with a 1× molar ratio of bispecific antibodies with two C-terminally linked anti-LNA antibody Fab fragment of the anti-LNA antibody produced by clone 1.9.21 Groups 7 and 8), or bispecific antibodies conjugated with ASO using the KTG technology (Groups 3, 4, 5, and 6). The ASO dose kept constant across all groups to enable comparison of ASO plasma PK between groups.

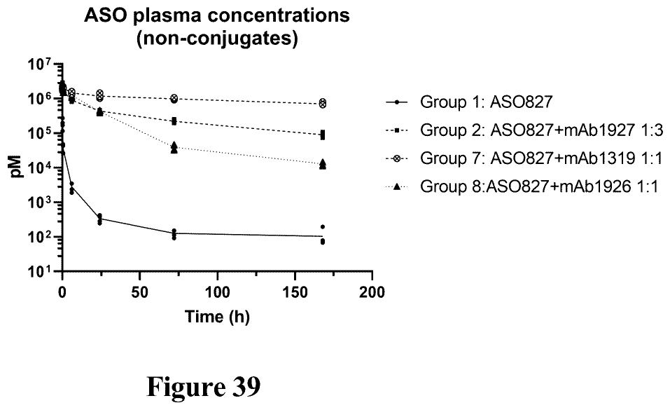

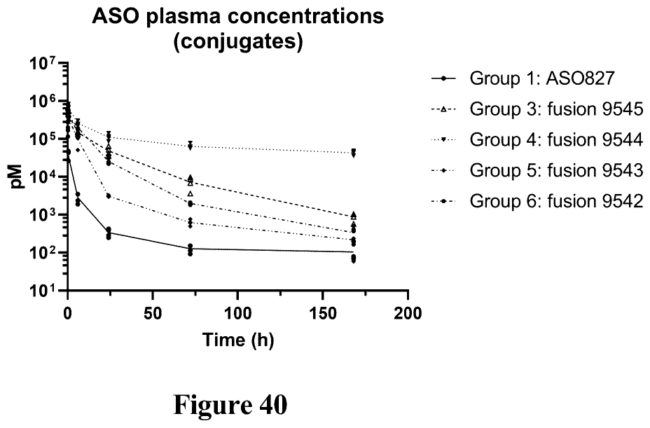

ASO levels in brain tissues were analyzed using the hELISA method as described in Example 13. Time dependent ASO levels in plasma samples were determined by back-calculating the OD values using a non-linear 4-parameter Rodbard-205 curve fitting function, with the standard calibration curve (naked ASO) prepared in assay buffer. The respective data is presented in FIGS. 39 and 40.

It can be seen that the addition of an LNA binding site to unconjugated as well as conjugated ASO had a profound impact on the PK of the ASO in complexed (non-conjugated) and conjugated form. Half-life extension was observed for non-covalently attached Fab and bsAb complexes (group 2 & 7 & 8), as well as for ASO-conjugates that harbored an additional anti-LNA antibody Fab fragment (group 4 & 6) compared to controls that did not contain a anti-LNA binding site.

### In Vivo Brain Exposure Study

To assess brain exposure of antibody-ASO complexes and conjugates, an in vivo single-dose plasma PK study was extended at its termination point to assess brain exposure in male C57BL/6J mice. The animal grouping and dosing details are summarized in Table 8-1. Mice were administered intravenously (i.v.) with the compounds (dose volume 5 mL/kg), including naked ASO (Group 1), ASO pre-incubated with a 3× molar ratio of IgG with one N-terminal anti-LNA antibody Fab fragment of the anti-LNA antibody produced by clone 1.9.21 (Group 2), ASO pre-incubated with a 1× molar ratio of bispecific antibodies with two C-terminally linked anti-LNA antibody Fab fragment of the anti-LNA antibody produced by clone 1.9.21 binding sites (Groups 7 and 8), or bispecific antibodies conjugated with ASO using the KTG technology (Groups 3, 4, 5, and 6). The ASO dose was kept constant across all groups (equivalent to 0.93 mg/kg of ASO 827) to enable comparison of ASO plasma PK and brain exposure between groups.

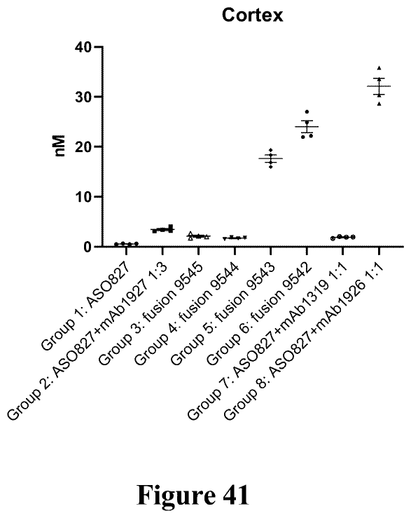

Brain tissues (cortex, cerebellum, rest of brain) were collected after termination of the study. ASO levels in brain tissues were analyzed using the hELISA method as described in Example 14. The respective data is presented in FIG. 41 (cortex), 42 (cerebellum) and 42 (rest of the brain).

The results show that the addition of an LNA binding site to unconjugated as well as conjugated ASO had an impact on the levels of ASO detectable in the brain. Increased ASO levels were observed in cortex, cerebellum and the rest of the brain for ASO which was non-covalently attached to a TfR-targeting anti-TfR/LNA bispüecific antibody (group 8) compared to controls that did not contain LNA binding sites. Increased ASO levels were also observed in cortex, cerebellum and the rest of the brain for ASO which was covalently conjugated to a TfR-targeting anti-TfR/LNA bispecific antibodies (group 6) compared to controls that did not contain LNA binding sites.

All publications, patents, and patent applications cited herein are hereby incorporated by reference herein in their entirety for all purposes to the same extent as if each individual publication, patent, and patent application were specifically and individually indicated to be so incorporated by reference. In the event that one or more of the incorporated literature and similar materials differs from or contradicts this application, including but not limited to defined terms, term usage, described techniques, or the like, this application controls.

The following examples and figures as well as the sequences are provided to aid the understanding of the present invention, the true scope of which is set forth in the appended claims. It is understood that modifications can be made in the procedures set forth without departing from the spirit of the invention.

That is, although the disclosed teachings have been described with reference to various applications, methods, and compositions, it will be appreciated that various changes and modifications can be made without departing from the teachings herein and the claimed invention below. The examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

### Example 1

### Immunization

LNA-binding antibodies were generated following four immunization strategies by hyperimmunization of different mouse strains (BALB/c and NMRI mice) with selected LNA-moiety containing ASOs (Table 1-1) coupled to keyhole limpet hemocyanine (KLH). Two different immunization schemes were applied, (a) immunization with a mixture of all three immunogens, and (b) alternating immunization with individual immunogens.

Positive immune responses in individual animals were checked by immunoassay titer analysis of sera using biotinylated versions of the three LNA immunogens and corresponding DNA controls (Table 1-2).

### Example 2

### Hybridoma Generation

From immunoreactive mice (2 animals selected), spleen cells were fused to Ag8 cells to generate antibody-producing fusion cells using state-of-the-art hybridoma cell technology. After cell fusion, hybridomas were screened for specific reactivity with LNA-containing ASOs using DNA-containing ASOs for specificity evaluation.

From two cell fusions, 3840 initial hybridoma clones resulting in 33 primary hybridoma clones were obtained. Thereof, 11 were selected for further characterization of binding properties by biomolecular interaction analysis (kinetic and thermodynamic surface plasmon resonance). Finally, 5 primary hybridoma clones were further processed by subcloning to generate monoclonal hybridoma cell culture clones (Tables 1-3 and 1-4).

### Example 3

### Expression and Purification of LNA-Binding Antibodies and -Derivatives and -Non-Binding Control Molecules

Expression of the antibodies according to the current invention in different formats was achieved by co-transfection of plasmids encoding light chain, heavy chain (with knob or hole mutations) and/or -fusion proteins (hole or knob) into mammalian cells (e.g. HEK293) via state of the art technologies.

In more detail, for example, for the production of such molecules by transient transfection (e.g. in HEK293 cells) expression plasmids based either on a cDNA organization with or without a CMV-Intron A promoter or on a genomic organization with a CMV promoter were applied.

### Beside the Antibody Expression Cassettes, the Plasmids Contained:

an origin of replication, which allows replication of this plasmid in E. coli,
a ß-lactamase gene, which confers ampicillin resistance in E. coli, and
a selectable marker in eukaryotic cells.

### The Transcription Unit of Each Antibody Gene was Composed of the Following Elements:

the immediate early enhancer and promoter from the human cytomegalovirus,
followed by the Intron A sequence in the case of the cDNA organization,
a 5′-untranslated region of a human antibody gene,
an immunoglobulin heavy chain signal sequence,
the antibody chain either as cDNA or in genomic organization,
a 3′-non-translated region with a polyadenylation signal sequence.

The fusion genes comprising the antibody chains were generated by gene synthesis and assembled by known recombinant methods and techniques by connection of the respective nucleic acid segments e.g. using unique restriction sites in the respective plasmids. The subcloned nucleic acid sequences were verified by DNA sequencing. For transient transfections larger quantities of the plasmids were prepared by plasmid preparation from transformed E. coli cultures (Nucleobond A X, Macherey-Nagel).

Standard cell culture techniques were used as described, e.g., in Current Protocols in Cell Biology (2000), Bonifacino, J. S., Dasso, M., Harford, J. B., Lippincott-Schwartz, J. and Yamada, K. M. (eds.), John Wiley & Sons, Inc.

The desired proteins were generated by transient transfection with the respective plasmid using the HEK293 system (ThermoFisher) according to the manufacturer's instruction. The antibodies were purified from cell culture supernatants by affinity chromatography using MabSelectSure-Sepharose™ (GE Healthcare, Sweden) or HiTrap KappaSelect-Agarose (Cytiva), followed by Superdex 200 size exclusion (GE Healthcare, Sweden) chromatography.

Briefly, sterile filtered cell culture supernatants were captured on a MabSelectSuRe or KappaSelect resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM sodium citrate at pH 3.0 (MabSelectSuRE) or at pH 2.7 (KappaSelect). The eluted antibody fractions were pooled and neutralized with 2 M Tris, pH 9.0. The antibody pools were further purified by size exclusion chromatography using a Superdex 200 16/60 GL (GE Healthcare, Sweden) column equilibrated with 20 mM histidine, 140 mM NaCl, pH 6.0. The ⅔-IgG containing fractions were pooled, concentrated to the required concentration using Vivaspin ultrafiltration devices (Sartorius Stedim Biotech S.A., France) and stored at −80° C.

Generally, expression yields between 3.6 mg/L and 70.3 mg/L with product purities of >90% (SEC analysis) were obtained. All molecules could be produced and purified to homogeneity. Quality control results of exemplary molecules are summarized in Table 9-1.

TABLE 9-1
Quality controls

| | | | Mass spec identify |
|---|---|---|---|
| Molecule | SEC (>90% | CE-SDS or | and composition |
| (identifier) | monomer) | SDS-PAGE | confirmed |
| mAb 5391 | X | X | X |
| mAb 5392 | X | X | X |
| mAb 5393 | X | X | X |
| fab 0699 | X | | X |
| fab 1988 | X | | |
| bsAb 1669 | X | | X |
| bsAb 1668 | X | | X |
| HC 5650 | X | X | X |
| mAb 3732 | X | X | X |
| mAb 4744 | X | X | X |
| mAb 0157 | X | X | X |
| mAb 0158 | X | X | X |
| mAb 0159 | X | X | X |
| mAb 0160 | X | X | X |
| mAb 4745 | X | X | X |
| mAb 0967 | X | X | X |
| mAb 0968 | X | X | X |
| mAb 0969 | X | X | X |
| mAb 0970 | X | X | X |
| bsAb 5439 | X | X | X |
| bsAb 5440 | X | X | X |
| bsAb 5441 | X | X | X |
| bsAb 5442 | X | X | X |
| bsAb 1870 | X | | |
| bsAb 1319 | X | X | |
| bsAb 6359 | X | X | |
| bsAb 7376 | X | X | |

The following table provides an overview of the structure of the molecules described herein.

TABLE 9-2
Molecules.

| | | N-terminal | C-terminal | | | |
|---|---|---|---|---|---|---|
| Molecule | core | to core | to core | Q- | position | |
| (identifier) | element | element | element | tag | Q-tag | ASO |
| mAb 5391 | IgG1 Fc- | 2x Fab from | − | − | − | − |
| recombinant | region | clone 1.2.8 | | | | |
| mAb 5392 | IgG1 Fc- | 2x Fab from | − | − | − | − |
| recombinant | region | clone 1.7.15 | | | | |
| mAb 5393 | IgG1 Fc- | 2x Fab from | − | − | − | − |
| recombinant | region | clone 1.9.21 | | | | |
| mAb 2002 | IgG1 Fc- | 1x anti-TfR | − | + | 297 of | − |
| recombinant | region | Fab 8D3 | | | full HC | |
| mAb 3732 | IgG1 Fc- | 1x anti-TfR | − | + | 297 of | − |
| recombinant | region | Fab 1026 | | | full HC | |
| mAb 5650 | IgG1 Fc- | 1x anti-TfR | − | + | 297 of | − |
| recombinant | region | Fab 1026 | | | full HC | |
| mAb 3708 | IgG1 Fc- | 1x DP47 Fab | − | − | − | − |
| recombinant | region | | | | | |
| mAb 4744 | IgG1 Fc- | 1x Fab from | − | − | − | − |
| recombinant | region | clone 1.9.21 | | | | |
| mAb 0157 | IgG1 Fc- | − | 8G2SG2 | − | − | − |
| recombinant | region | | linker - Fab | | | |
| | | | from clone | | | |
| | | | 1.9.21 | | | |
| mAb 0158 | IgG1 Fc- | 1x Fab from | same as N- | − | − | − |
| recombinant | region | clone 1.9.21 | terminal Fab - | | | |
| | | | 8G2SG2 | | | |
| | | | linker - Fab | | | |
| | | | from clone | | | |
| | | | 1.9.21 | | | |
| mAb 0159 | IgG1 Fc- | − | 4G2SG2 | − | − | − |
| recombinant | region | | linker - Fab | | | |
| | | | from clone | | | |
| | | | 1.9.21 | | | |
| mAb 0160 | IgG1 Fc- | 1x Fab from | same as N- | − | − | − |
| recombinant | region | clone 1.9.21 | terminal Fab - | | | |
| | | | 4G2SG2 | | | |
| | | | linker - Fab | | | |
| | | | from clone | | | |
| | | | 1.9.21 | | | |
| mAb 4745 | IgG1 Fc- | 1x DP47 Fab | − | − | − | − |
| FORCE | region | | | | | |
| mAb 0967 | IgG1 Fc- | − | same as N- | − | − | − |
| FORCE | region | | terminal Fab - | | | |
| | | | 8G2SG2 | | | |
| | | | linker - | | | |
| | | | DP47 Fab | | | |
| mAb 0968 | IgG1 Fc- | 1x DP47 Fab | same as N- | − | − | − |
| FORCE | region | | terminal Fab - | | | |
| | | | 8G2SG2 | | | |
| | | | linker - | | | |
| | | | DP47 Fab | | | |
| mAb 0969 | IgG1 Fc- | − | same as N- | − | − | − |
| FORCE | region | | terminal Fab - | | | |
| | | | 4G2SG2 | | | |
| | | | linker - | | | |
| | | | DP47 Fab | | | |
| mAb 0970 | IgG1 Fc- | 1x DP47 Fab | same as N- | − | − | − |
| FORCE | region | | terminal Fab - | | | |
| | | | 4G2SG2 | | | |
| | | | linker - | | | |
| | | | DP47 Fab | | | |
| fab 0669 | − | 1x Fab from | − | − | − | − |
| recombinant | | clone 1.9.21 | | | | |
| fab 1988 | − | 1x DP47 Fab | − | − | − | − |
| recombinant | | | | | | |
| bsAb 1669 | IgG1 Fc- | 1x Fab from | same as N- | + | 297 of | − |
| recombinant | region | clone 1.9.21 | terminal Fab - | | full HC | |
| | | | 1x scFab | | | |
| | | | 1026 | | | |
| bsAb 1668 | IgG1 Fc- | 1x Fab from | same as N- | + | 297 of | − |
| recombinant | region | clone 1.9.21 | terminal Fab - | | full HC | |
| | | | 1x scFab | | | |
| | | | 8D3 | | | |
| bsAb 4318 | IgG1 Fc- | 1x Fab from | different as | + | 297 of | 576 |
| recombinant | region | clone 1.9.21 | N-terminal | | full HC | |
| | | | Fab - 1x | | | |
| | | | scFab 8D3 | | | |
| bsAb 4319 | IgG1 Fc- | 1x Fab from | different as | + | 297 of | 576 |
| recombinant | region | clone 1.9.21 | N-terminal | | full HC | |
| | | | Fab - 1x | | | |
| | | | scFab 1026 | | | |
| bsAb 5439 | IgG1 Fc- | 1x Fab 1026 | different as | + | 297 of | − |
| recombinant | region | | N-terminal | | full HC | |
| | | | Fab - | | | |
| | | | 4G2SG2 | | | |
| | | | linker - 1x | | | |
| | | | DP47 Fab | | | |
| bsAb 5440 | IgG1 Fc- | 1x Fab 1026 | different as | + | 297 of | − |
| recombinant | region | | N-terminal | | full HC | |
| | | | Fab - | | | |
| | | | 4G2SG2 | | | |
| | | | linker - 1x | | | |
| | | | Fab from | | | |
| | | | clone 1.9.21 | | | |
| bsAb 1319 | IgG1 Fc- | 1x Fab nada | 2 x: | + | 297 of | − |
| recombinant | region | | 4G2SG2 | | full HC | |
| | | | linker - 1x | | | |
| | | | Fab from | | | |
| | | | clone 1.9.21 | | | |
| bsAb 6359 | IgG1 Fc- | 1x Fab 1026 | 2 x: | + | 297 of | − |
| recombinant | region | | 2G2SG2 | | full HC | |
| | | | linker - 1x | | | |
| | | | Fab from | | | |
| | | | clone 1.9.21 | | | |
| bsAb 7376 | IgG1 Fc- | 1x Fab 8D3 | 2 x: | + | 297 of | − |
| recombinant | region | | 4G2SG2 | | full HC | |
| | | | linker - 1x | | | |
| | | | Fab from | | | |
| | | | clone 1.9.21 | | | |
| bsAb 5441 | IgG1 Fc- | 1x DP47 Fab | different as | + | 297 of | − |
| recombinant | region | | N-terminal | | full HC | |
| | | | Fab - | | | |
| | | | 4G2SG2 | | | |
| | | | linker - 1x | | | |
| | | | DP47 Fab | | | |
| bsAb 5442 | IgG1 Fc- | 1x DP47 Fab | different as | + | 297 of | − |
| recombinant | region | | N-terminal | | full HC | |
| | | | Fab - | | | |
| | | | 4G2SG2 | | | |
| | | | linker - 1x | | | |
| | | | Fab from | | | |
| | | | clone 1.9.21 | | | |
| bsAb 1870 | IgG1 Fc- | 1x DP47 Fab | 2 x: | + | 297 of | − |
| recombinant | region | | 2G2SG2 | | full HC | |
| | | | linker - 1x | | | |
| | | | Fab from | | | |
| | | | clone 1.9.21 | | | |
| bsAb 1861 | IgG1 Fc- | 1x DP47 Fab | 2 x: | + | 297 of | − |
| recombinant | region | | 4G2SG2 | | full HC | |
| | | | linker - 1x | | | |
| | | | Fab from | | | |
| | | | clone 1.9.21 | | | |
| fusion 2489 | IgG1 Fc- | 1x anti-TfR | − | + | 297 of | 420 |
| recombinant | region | Fab 1026 | | | full HC | |
| fusion 2899 | IgG1 Fc- | 1x anti-TfR | − | + | 297 of | 420 |
| recombinant | region | Fab 8D3 | | | full HC | |
| fusion 2556 | IgG1 Fc- | 1x anti-TfR | − | + | 446 of | 918 |
| recombinant | region | Fab 1026 | | | full HC | |
| fusion 2555 | IgG1 Fc- | 1x anti-TfR | − | + | 341 of | 918 |
| recombinant | region | Fab 1026 | | | full HC | |
| fusion 2554 | IgG1 Fc- | 1x anti-TfR | − | + | 297 of | 918 |
| recombinant | region | Fab 1026 | | | full HC | |
| fusion 6431 | IgG1 Fc- | 1x anti-TfR | − | + | 297 of | 576 |
| FORCE | region | Fab 1026; 1x | | | full HC | |
| | | DP47 Fab | | | | |
| fusion 6430 | IgG1 Fc- | 1x anti-TfR | − | + | 297 of | 576 |
| FORCE | region | Fab 1026; 1x | | | full HC | |
| | | Fab from | | | | |
| | | clone 1.9.21 | | | | |
| fusion 1511 | IgG1 Fc- | 1x anti-TfR | different as | + | 297 of | 576 |
| FORCE | region | Fab 1026 | N-terminal | | full HC | |
| | | | Fab - | | | |
| | | | 8G2SG2 | | | |
| | | | linker - 1x | | | |
| | | | DP47 Fab | | | |
| fusion 1507 | IgG1 Fc- | 1x anti-TfR | different as | + | 297 of | 576 |
| FORCE | region | Fab 1026 | N-terminal | | full HC | |
| | | | Fab - | | | |
| | | | 8G2SG2 | | | |
| | | | linker - 1x | | | |
| | | | Fab from | | | |
| | | | clone 1.9.21 | | | |
| fusion 1512 | IgG1 Fc- | 1x anti-TfR | same as N- | + | 297 of | 576 |
| FORCE | region | Fab 1026; 1x | terminal | | full HC | |
| | | DP47 Fab | DP47 Fab - | | | |
| | | | 8G2SG2 | | | |
| | | | linker - 1x | | | |
| | | | DP47 Fab | | | |
| fusion 1508 | IgG1 Fc- | 1x anti-TfR | same as N- | + | 297 of | 576 |
| FORCE | region | Fab 1026; 1x | terminal Fab | | full HC | |
| | | Fab from | of clone | | | |
| | | clone 1.9.21 | 1.9.21 - | | | |
| | | | 8G2SG2 | | | |
| | | | linker - 1x | | | |
| | | | Fab from | | | |
| | | | clone 1.9.21 | | | |
| fusion 1513 | IgG1 Fc- | 1x anti-TfR | different as | + | 297 of | 576 |
| FORCE | region | Fab 1026 | N-terminal | | full HC | |
| | | | Fab - | | | |
| | | | 4G2SG2 | | | |
| | | | linker - 1x | | | |
| | | | DP47 Fab | | | |
| fusion 1509 | IgG1 Fc- | 1x anti-TfR | different as | + | 297 of | 576 |
| FORCE | region | Fab 1026 | N-terminal | | full HC | |
| | | | Fab - | | | |
| | | | 4G2SG2 | | | |
| | | | linker - 1x | | | |
| | | | Fab from | | | |
| | | | clone 1.9.21 | | | |
| fusion 1514 | IgG1 Fc- | 1x anti-TfR | same as N- | + | 297 of | 576 |
| FORCE | region | Fab 1026; 1x | terminal | | full HC | |
| | | DP47 Fab | DP47 Fab - | | | |
| | | | 4G2SG2 | | | |
| | | | linker - 1x | | | |
| | | | DP47 Fab | | | |
| fusion 1510 | IgG1 Fc- | 1x anti-TfR | same as N- | + | 297 of | 576 |
| FORCE | region | Fab 1026; 1x | terminal Fab | | full HC | |
| | | Fab from | of clone | | | |
| | | clone 1.9.21 | 1.9.21 - | | | |
| | | | 4G2SG2 | | | |
| | | | linker - 1x | | | |
| | | | Fab from | | | |
| | | | clone 1.9.21 | | | |
| fusion 0852 | IgG1 Fc- | 1x anti-TfR | different as | + | 297 of | 049 |
| recombinant | region | Fab 1026 | N-terminal | | full HC | |
| | | | Fab - | | | |
| | | | 4G2SG2 | | | |
| | | | linker - 1x | | | |
| | | | DP47 Fab | | | |
| fusion 0853 | IgG1 Fc- | 1x anti-TfR | different as | + | 297 of | 049 |
| recombinant | region | Fab 1026 | N-terminal | | full HC | |
| | | | Fab - | | | |
| | | | 4G2SG2 | | | |
| | | | linker - 1x | | | |
| | | | Fab from | | | |
| | | | clone 1.9.2 | | | |
| fusion 0854 | IgG1 Fc- | 1x DP47 Fab | different as | + | 297 of | 049 |
| recombinant | region | | N-terminal | | full HC | |
| | | | Fab - | | | |
| | | | 4G2SG2 | | | |
| | | | linker - 1x | | | |
| | | | DP47 Fab | | | |
| fusion 0855 | IgG1 Fc- | 1x DP47 Fab | different as | + | 297 of | 049 |
| recombinant | region | | N-terminal | | full HC | |
| | | | Fab - | | | |
| | | | 4G2SG2 | | | |
| | | | linker - 1x | | | |
| | | | Fab from | | | |
| | | | clone 1.9.21 | | | |
| fusion 3918 | IgG1 Fc- | 1x <nada> | different as | + | 297 of | 049 |
| recombinant | region | Fab | N-terminal | | full HC | |
| | | | Fab - | | | |
| | | | 4G2SG2 | | | |
| | | | linker - 1x | | | |
| | | | DP47 Fab | | | |
| fusion 3919 | IgG1 Fc- | 1x <nada> | different as | + | 297 of | 049 |
| recombinant | region | Fab | N-terminal | | full HC | |
| | | | Fab - | | | |
| | | | 4G2SG2 | | | |
| | | | linker - 1x | | | |
| | | | Fab from | | | |
| | | | clone 1.9.21 | | | |
| fusion 3920 | IgG1 Fc- | 1x <nada> | 2x: 4G2SG2 | + | 297 of | 049 |
| recombinant | region | Fab | linker - | | full HC | |
| | | | DP47 Fab | | | |
| fusion 3921 | IgG1 Fc- | 1x <nada> | 2x: 4G2SG2 | + | 297 of | 049 |
| recombinant | region | Fab | linker - Fab | | full HC | |
| | | | from clone | | | |
| | | | 1.9.21 | | | |
| mAb 1927 | IgG1 Fc- | 1x Fab from | − | − | − | − |
| recombinant | region | clone 1.9.21 | | | | |
| fusion 9545 | IgG1 Fc- | 1x <nada> | different as N- | + | 297 of | 385 |
| recombinant | region | Fab | terminal Fab - | | full HC | |
| | | | 4G2SG2 | | | |
| | | | linker - 1x | | | |
| | | | DP47 Fab | | | |
| fusion 9544 | IgG1 Fc- | 1x <nada> | different as N- | + | 297 of | 385 |
| recombinant | region | Fab | terminal Fab - | | full HC | |
| | | | 4G2SG2 | | | |
| | | | linker - 1x | | | |
| | | | Fab from | | | |
| | | | clone 1.9.21 | | | |
| fusion 9543 | IgG1 Fc- | 1x anti-TfR | different as N- | + | 297 of | 385 |
| recombinant | region | Fab 8D3 | terminal Fab - | | full HC | |
| | | | 4G2SG2 | | | |
| | | | linker - 1x | | | |
| | | | DP47 Fab | | | |
| fusion 9542 | IgG1 Fc- | 1x anti-TfR | different as N- | + | 297 of | 385 |
| recombinant | region | Fab 8D3 | terminal Fab - | | full HC | |
| | | | 4G2SG2 | | | |
| | | | linker - 1x | | | |
| | | | Fab from | | | |
| | | | clone 1.9.21 | | | |
| bsAb 1319 | IgG1 Fc- | 1x <nada> | 2 x: 4G2SG2 | + | 297 of | − |
| recombinant | region | Fab | linker - Fab | | full HC | |
| | | | from clone | | | |
| | | | 1.9.21 | | | |
| bsAb 1926 | IgG1 Fc- | 1x anti-TfR | 2 x: 4G2SG2 | + | 297 of | − |
| recombinant | region | Fab 8D3 | linker - Fab | | full HC | |
| | | | from clone | | | |
| | | | 1.9.21 | | | |

−: not present;
+: present;
numbering according to Kabat;
full HC = part of the IgG Fc-region conjugated to Fab;
recombinant: produced by recombinant expression;
FORCE: produced by FORCE technology

### Example 4

Antibodies according to the current invention conjugated to an LNA-modified ASO

To generate antibody with an ASO payload, Kutzneria albida Transglutaminase (KTG)-mediated site-directed conjugation combined with copper-free click reaction to attach ASO on antibodies was applied as described in WO 2023/118398.

Briefly, antibody tagged with the amino acid sequence YRYRQ (Q-tag; SEQ ID NO: 134) was first enzymatically linked with a molar excess of an azide containing linker tagged with an amino acid sequence RYESK (K-tag; SEQ ID NO: 136) in histidine/NaCl buffer using KTG at 37° C. Desired products were separated from enzyme and unreacted educts using a Superdex™ 200 increase 10/300 GL size exclusion column. In the second step, a molar excess of an ASO tagged with a BCN group was attached to the antibody-azide linker through a copper-free click reaction in PBS supplemented with Arginine at pH 7.4. Desired products were separated from unreacted educts using a Superdex™ 200 increase 10/300 GL size exclusion column. Conjugation efficiency and the molecular composition were confirmed by mass spectrometry.

Attachment of ASO to a bispecific anti-TfR/LNA(from clone 1.9.21) antibody (<TfR><LNA3> bispecific antibody; FIG. 20) resulted in the formation of dimers or multimers, which appear as separate peaks before the fraction that contains the monomeric antibody on an analytic size exclusion chromatography (SEC-HPLC; FIG. 21). The composition of molecules was further confirmed by mass spectrometry-based methods.

Briefly, samples were deglycosylated by adding N-Glycosidase F (Roche Diagnostics, Penzberg, Germany) before the measurements. The deglycosylation was performed in sodium phosphate buffer at pH 7.1, at a ratio of 0.14 U/μg antibody. The reaction mixture was incubated for 16 h at 37° C., and samples were subsequently separated by reverse-phase chromatography (RP) or size exclusion chromatography (SEC). RP was performed using a PLRP-S column (Agilent, Waldbronn, Germany) with mobile phase A containing 0.1% (v/v) formic acid in UPLC grade water, and mobile phase B containing acetonitrile (Fischer Chemical, Schwerte, Germany). The column temperature was 75° C., and a gradient of mobile phase B was used for separation. MS spectra were acquired using a MaXis Q-TOF instrument (Bruker Daltonics, Bremen, Germany) controlled by Compass 6.2 software. SEC was performed using an Acquity Premier SEC column (4.6×300 mm, 1.7 μm particle size; Waters) and an isocratic elution using 200 mM CH3COONH4 at 250 μl/min. Before electrospray ionization using the Nanospray Flex ion source, a Flow Split 1/100 was used. MS spectra were acquired using a Thermo Scientific UHMR mass spectrometer (Thermo Fisher Scientific) controlled by Xcalibur 4.5 software. For data evaluation, in-house-developed software was used.

Native mass spec analysis (SEC-MS) confirmed those peaks contain dimers. Further mass spectrometric analysis under denaturing condition (RP-MS) revealed a lower than expected conjugation efficiency with an average DAR of 0.7-0.8 (Table 7-1). Without being bound by this theory, it is assumed that these results suggest that conjugation of ASO to a non-optimized format of <TfR><LNA3> bispecific antibodies, <LNA3> binder drives the formation of dimers and interferes with the ASO conjugation.

Format chain exchange (FORCE) technology was applied for the generation of bispecific antibodies in combinatorial binder-format matrices (see, e.g., Dengl et al. Nat Commun 11, 4974 2020, WO 2019/077092) to identify the preferred format of <TfR><LNA> bispecific antibody conjugated with the ASO payload.

Briefly, ASO was attached to the FORCE educt antibody containing a <hTfR> binder but without a <LNA> binder from clone 1.9.21. Mass spec analysis of these samples under denaturing condition (RP-MS) confirmed the successful conjugation with an average DAR close to 1 (FIG. 22; Table 7-2). In order to start the exchange reaction, educt containing a <hTfR> binder with ASO payload were mixed with equimolar amounts of educt containing <LNA3> binder from clone 1.9.21 or <DP47> non-binder in different formats (FIG. 23) at a total protein concentration of 1 mg/ml in 1×PBS supplemented with 250 mM Arginine and 0.05% Tween 20 with 20× molar ratio of TCEP at 37° C. for 3 hr. Unreacted educts and aggregates were removed by a CaptureSelect™ C-tagXL Pre-packed Column (1 ml or 5 ml, Thermo Scientific). The resulting FORCE products were analyzed by size exclusion chromatography (SEC-HPLC) with BioSuite High Resolution SEC Columns (250 Å, 5 μm, Waters, USA) using a 200 mM K₂HPO₄/KH₂PO₄, 250 mM KCl, pH 7.0 running buffer at a flow rate of 0.5 ml/min to determine the purity of the bispecific product formation. The results are shown in FIG. 23 and unexpectedly revealed that the N-terminally linked <LNA3> binder leads to the formation of high molecular weight products and that, on the other hand, a single C-terminally linked <LNA3> binder with a flexible GS linker (20 amino acid or 40 amino acid) leads to products with >90% of monomer. Products with <DP47> non-binders instead of <LNA3> binders, regardless of formats, formed >90% of monomer, confirming that an N-terminally linked <LNA3> binder is the cause of high molecular weight products formation.

After identifying the preferred format of <TfR><LNA> bispecific antibody with the FORCE technology, the same format of bispecific antibody conjugated with ASO payload were generated with conventional recombinant approach, i.e. recombinant expression of the bispecific antibody and KTG-mediated site-directed conjugation combined with copper-free click reaction. The resulting molecules formed predominantly monomers (FIG. 26). Mass spectrometric analysis also revealed a good conjugation efficiency, confirming the good biophysical behavior of the identified format (Table 7-3).

### Example 5

### Binding Properties

The selected monoclonal LNA-specific Abs were further characterized for binding characteristics and epitope mapped by immunoassay using capture molecules containing the following binding epitopes and combinations thereof
LNA (2′-O.4-C-methylene)
phosphorothioate in the NA backbone
nucleotides/sequence of bases
DNA
GalNAc-C6-modification

The results of the antibody characterization by solid phase immunoassay (ELISA) using different biotinylated LNA, PS-backbone or DNA-containing NA-oligonucleotides are shown in FIGS. 1 to 5. The binding properties in immunoassays are summarized in Table 3-1.

### Example 6

### X-Ray Structure of LNA-Binding Antibodies According to the Current Invention

Crystallization, data collection and structure determination of anti-LNA antibody produced by clone 1.9.21 in Fab format (fab 0699) in complex with LNA tCac (ASO 980) was carried out.

Fab fragment 0699 was concentrated to 24.6 mg/ml. Crystal screening was performed at 21° C. in sitting drop vapor diffusion experiments using a drop sizes of 200 nL with 50% and 70% (v/v) amount of protein. Several crystal hits were identified out of the Protein Complex Suite (Qiagen) and BCS (Molecular Dimensions Ltd.) screens. Plate shaped crystals with a size of approximately 200 μm×70 μm×10 μm appeared out of 0.1 M HEPES buffer of pH 7.0 supplemented with 20% PEG8000 within eight days after setup of the experiment. The complex with LNA was obtained by soaking crystals for 16 hours in a solution of 2 mM of ASO 980. The soaking solution was prepared from a 20 mM stock in water of ASO 980 which was subsequently diluted with crystallization solution to reach the final soaking concentration of 2 mM.

For data collection crystals were flash cooled at 100 K in crystallization solution supplemented with 20% ethylene glycol. X-ray diffraction data were collected at a wavelength of 0.999991 Å using an Eiger 2 16 M detector at the beamline X10SA of the Swiss Light Source (Villigen, Switzerland). Data have been processed with XDS (Kabsch, W., XDS. Acta Cryst. D66, 125-132 (2010)), scaled with AIMLESS (P. R. Evans and G. N. Murshudov, Acta Cryst. (2013). D69, 1204-1214) and analyzed for anisotropy with STARANISO (Tickle, I. J., Flensburg, C., Keller, P., Paciorek, W., Sharff, A., Vonrhein, C., Bricogne, G. (2018). STARANISO (https://staraniso.globalphasing.org/cgi-bin/staraniso.cgi); Cambridge, United Kingdom: Global Phasing Ltd.). The crystals of the complex belong to space group C2 with cell axes of a=126.61 Å, b=59.60 Å, c=62.50 Å with P=94.30° and diffract to a resolution of 1.57 Å. The structure was determined by molecular replacement with PHASER (McCoy, A. J., Grosse-Kunstleve, R. W., Adams, P. D., Winn, M. D., Storoni, L. C., & Read, R. J. Phaser crystallographic software. J. Appl. Cryst. 40, 658-674 (2007)) using the coordinates of an in house Fab as search model. Molecular replacement/determination of the structure can be done with any Fab fragment coordinates which are closely related in sequence to the target and are available in the Protein databank (rcsb.org). Could also be done with a replacement model generated from sequence via Alphafold.

Difference electron density guided the exchange of amino acids according to the sequence differences to the search model and for model building of the LNA. The structure was refined with programs from the CCP4 suite (Winn, M. D. et al., Acta. Cryst. D67, 235-242 (2011)) and BUSTER (Bricogne, Blanc, G. E., Brandl, M., Flensburg, C., Keller, P., Paciorek, W., Roversi, P., Sharff, A., Smart, O. S., Vonrhein, C., Womack, T. O. Buster version 2.9.5 Cambridge, United Kingdom: Global Phasing Ltd. (2011)). Manual rebuilding was done with COOT (Emsley, P., et al., Acta Cryst. D66, 486-501 (2010)). Data collection and refinement statistics are summarized in Table 6-1. All graphical presentations were prepared with PYMOL (The Pymol Molecular Graphics System, Version 1.7.4. Schrödinger, LLC.)

### Example 7

### Binding Characterization by SPR

To assess the specificities and affinities of LNA-binding antibodies, surface plasmon resonance was done as described in WO 2020/141117 and WO 2022/175217. ASO binding was investigated by surface plasmon resonance (SPR) using a BIAcore T200 instrument (Cytiva). As a running and dilution buffer, HEPES buffered saline (HBS) containing 0.05% PS20 was used, the temperature was set to 25° C. For capturing anti-LNA Fabs or complete anti-LNA antibodies to a Series S Sensor Chip CM3 (Cytiva), either a mouse anti-human IgG antibody (Roche Diagnostics GmbH) or a goat anti-human F(ab′2) antibody (Jackson ImmunoResearch) was immobilized using standard amine coupling chemistry with final total surface densities of approximately 5000 resonance units (RU). The anti-LNA antibodies were captured to the surface by an injection for 30 sec., leading to response levels of approx. 100-200 RU. Different ASO molecules were injected in a 1:3 dilution series up to 1000 nM. Association was monitored for 3 min. and dissociation for 5 min. at a flow rate of 30 μl/min each. Subsequently, the surface was regenerated by injecting 10 mM NaOH (anti-human IgG antibody surface) or 10 mM Glycine pH 1.7 (anti-human IgG F(ab′2) antibody surface) for 60 sec. Bulk refractive index differences were corrected by subtracting blank injections and by subtracting the response obtained from the reference flow cell without captured antibody. Curve fitting was performed using the 1:1 Langmuir binding model within the BIAcore evaluation software.

To compare the three <LNA> binders (anti-LNA antibodies or Fabs) from clones 1.2.8, 1.7.15 and 1.9.21, the binding kinetics and affinities of different LNA-modified ASOs or antibody-ASO conjugates applied as analytes were determined towards <LNA1>(clone 1.2.8), <LNA2>(clone 1.7.15) and <LNA3>(clone 1.9.21) in bivalent standard Y-shaped IgG format. The results of these analyses are shown in Table 3-2 and example sensorgrams are shown in FIGS. 6 to 8. <LNA1> shows no to very low binding to all of the analytes tested. <LNA2> binds to some of the ASOs, while showing no or very low binding to other ASOs. <LNA3> binds to all of the tested ASOs and antibody-ASO conjugates with good affinity.

To further characterize the <LNA3> binder and avoid the bivalent avidity effect, <LNA3> was produced in monovalent Fab and C-terminally linked monovalent formats. Their binding kinetics and affinities towards different LNA-modified ASOs as well as one siRNA were tested. The results are summarized in Table 3-3 and example sensorgrams are shown in FIGS. 9 to 12. Confirming the results obtained from the bivalent IgG format, <LNA3> as the monovalent formats binds to all of the tested single-stranded LNA-modified ASOs, irrespective of defined composition and sequences. However, it does bind to siRNA, which is double-stranded and not LNA-modified.

Further analyses of the universal <LNA3> binder in the bivalent <DP47>/2×C-<LNA3> format (IgG1 Fc-region with one DP47 Fab conjugated to one N-terminus of the Fc-region and an anti-LNA Fab from clone 1.9.21 conjugated to each C-terminus of the Fc-region) revealed 1:1 antibody-to-analyte ratio of binding with the Kd at single digit pico-molar range (Table 3-4). This indicates that ASOs are bound in an avidity-driven manner, i.e. two binding arms of the <LNA3> binders can bind to one, i.e. the same, 16- to 20-mer ASO. Thus, LNA-modified ASOs of sufficient lengths can be bound by more than one <LNA3> binding arm of the same antibody.

To characterize the detailed binding properties of <LNA3>, we assessed its binding to tetramer ASOs that contain various modifications (LNA and phosphorothioate bond) and/or sequences at different positions. The results of these analyses are listed in Table 3-5 and reveal several binding characteristics:
unmodified ASO is not bound, at least one phosphorothioate bond or one LNA-modified sugar must be present;
1^{st} base preferentially to be C or T or G=not A;
LNA-modified sugar preferable at the 2nd position;
more LNA-modification correlates with higher affinity;
phosphorothioate bond located only between the second and the third nucleotide is not preferred.

### Example 8

### Direct ADA Assay

In one exemplary setup, a mouse-human chimeric version of the LNA-specific mAb according to the current invention produced by clone 1.9.21 (mAb<LNA>Chim-1.9.21-IgG) was used as ADA positive control in an exemplary direct ADA assay.

Shortly, a mixture of two biotinylated drug molecules (5′-Bi-LNA/3′-Bi-LNA; BI=biotin) is used as capture moiety in a serial sandwich ELISA. In a first step, a streptavidin (SA) coated microtiter plate (SA-MTP) is contacted with the biotinylated capture molecules 5′-Bi-LNA/3′-Bi-LNA and incubated for 1 h at RT on a microtiter plate (MTP) shaker. After coating and washing, test samples, positive control mAb<LNA>Chim-1.9.21-IgG and positive control samples are added to the coated microtiter plate and incubated for 1 h to immobilize ADA-drug complexes via the immobilized capture molecules. Again, following aspiration of the supernatant unbound substances are removed by three-fold repeated washings. For detection, immobilized ADA-drug complexes are successively incubated with a monoclonal anti-human-IgG-antibody-Dig conjugate (mAb<h-Fc-pan>M-R10Z8E9-IgG-Dig; Dig=digoxygenin) and a polyclonal anti-digoxygenin Fab fragments conjugated to horseradish peroxidase (anti-digoxygenin-POD (poly)). Finally, the generated peroxidase is visualized by ABTS substrate solution resulting in the formation of a colored reaction product. The color intensity which is photometrically determined at 405 nm (490 nm reference wavelength) is proportional to the ADA analyte concentration in the plasma sample.

A typical calibration curve of the positive control and corresponding signal data of the assay is shown in FIG. 16 and Table 4-1.

### Example 9

### LNA-Containing Drug Detection Assay

Application of LNA-specific antibody from clone 1.9.21 as capture antibody in combination with a human IgG specific detection antibody for the quantitative detection of antibody-ASO conjugate drug.

The quantification of the antibody-antisense oligonucleotide (ASO) conjugates was carried out using a sandwich enzyme-linked immunosorbent assay (ELISA). Initially, antibody-ASO conjugates and diluted plasma samples were prepared at double concentration (2×) in a pre-dilution plate. The first detection antibody (digoxygenin (DIG) label) targeting the Fc-region of the human IgG was also prepared at a 2× concentration in a separate pre-dilution plate. Hybridization was initiated by transferring the calibrator/sample pre-dilution plate to the detection antibody plate, followed by a one-hour incubation with gentle shaking. The biotin-labeled LNA-specific capture antibody was immobilized on a streptavidin-coated microtiter plate (SA-MTP) by adding 100 μl at 10 ng/ml to each well and incubating for one hour with gentle shaking. After washing the SA-MTP, the hybridized solution was transferred to the plate with the immobilized capture antibody and incubated for an additional hour with gentle shaking. After washing, the second detection antibody, anti-digoxygenin-antibody POD conjugate (POD=peroxidase), was added and incubated for one hour with gentle shaking. Visualization of the immobilized hybridized complexes was achieved by adding BM Blue (TMB) solution, with the optical density (OD) measured at 680 nm (reference wavelength 450 nm) under gentle shaking until a maximum of 0.7 OD was reached, typically within 5-30 minutes. The reaction was terminated by adding 50 μl of 1 M H2SO4, causing the reaction product to turn yellow. Endpoint measurement was performed at 450 nm (reference 690 nm), with the highest standard reaching a maximum of 2.2 OD. The quantification of the antibody-ASO conjugate was performed by back-calculating the OD values using a non-linear 4-parameter Rodbard-205 curve fitting function, with the standard calibration curve prepared in hybridization buffer.

### Example 10

Functional Cellular Uptake Assay with an Anti-TfR/LNA Antibody According to the Current Invention

To show the functionality of the anti-LNA antibodies according to the current invention in cellular assays, a colocalization assay was done using the human blood-brain-barrier endothelial cell line hCMEC/D3.

Briefly, hCMEC/D3 cells were maintained in EBM-2 Basal Medium (Lonza, #CC-3156) supplemented with EGM-2 MV SingleQuots (Lonza, #CC-4147), but using only a fraction of the total volume of the growth factors (IGF, VEGF, EGF, FGF) provided and of the provided FBS (10%) and with complete hydrocortisone, ascorbic acid and gentamycin. About 3-4 days before the treatment, cells were plated in ibidi chamber (Cat. No: 80827) coated with 50 μg/ml collagen (BD Biosciences #354236) at a density of 15,000 cells/cm². 30 nM of <TfR>(anti-TfR antibody) alone, <TfR>—ASO conjugate (anti-TfR antibody conjugated to ASO with KTG and click chemistry), and <TfR>—ASO conjugate pre-incubated with different molar ratio of <LNA3> Fab or DP47 Fab were applied to cells in the complete culture media for 3 h at 37° C. In the last 20 min. of incubation, Alexa555-transferrin (ThermoFischer #T35352) was applied to cells at a final concentration of 7.5 μg/ml to label the transferrin receptor. Cells were then washed twice with PBS+0.1% Heparin before fixation with 4% PFA (paraformaldehyde). After fixation, cells were permeabilized with buffer containing 16.7% goat serum, 0.3% Triton X-100, 10 mM Na₂HPO₄, 10 mM NaH₂PO₄, and 450 mM NaCl for 30 min and IgGs were labeled by a goat anti-human IgG antibody, Fcγ fragment specific secondary antibody (Jackson ImmunoResearch #109-546-170) for 1 h at RT. Images were acquired on a Leica SP5 confocal microscopy. An object-based colocalization analysis between IgG and transferrin receptor was carried out using a customized workflow in Cell Profiler. Results are shown in FIG. 27 and reveal that conjugation of ASO to <TfR> binding antibody reduced the IgG colocalization with transferrin receptor, indicating the ASO payload contributes to transferrin receptor-independent uptake. However, pre-incubate the <TfR>—ASO conjugate with <LNA3> Fab, but not DP47 Fab rescued the IgG colocalization with the transferrin receptor in a dose-dependent manner, indicating the addition of <LNA3> binder reduces the transferrin receptor-independent uptake.

To access functionality of <LNA3> as a covalently-linked intramolecular binder, <TfR/LNA3> bsAb (anti-TfR/LNA from clone 1.9.21 bispecific antibody) conjugated with ASO payload and the corresponding controls (<DP47/LNA3>−ASO) generated by FORCE or by the conventional recombinant method with enzymatic conjugation were tested in the colocalization assay. Results are shown in FIG. 28 and FIG. 29, respectively. The assay revealed that the C-terminally linked <LNA3> binder improves colocalization with the transferrin receptor as compared to the corresponding control molecules with a <DP47>. This indicates that a C-terminally linked <LNA3> binder improves the transferrin receptor-mediated uptake or recycle. Of note, the same molecules also formed predominantly monomers in HPLC-SEC.

To test if the <LNA3> binder may mediate the uptake of ASO in a non-covalent manner, the format with two C-terminally linked <LNA3> was used since SPR data revealed that this format can drive 1:1 antibody-to-ASO ratio of binding with high avidity (kD around 3 μM). <LNA3> containing IgG were premixed with the Atto647N-linked ASO (Microsynth) at 1:1 molar ratio at RT for 30 min. before applying to hCMEC/D3 cells at a concentration of 30 nM for 3 h at 37° C. In the last 20 min. of incubation, Alexa555-transferrin (ThermoFischer #T35352) was applied to cells at a final concentration of 7.5 μg/ml to label the transferrin receptor. Cells were fixed by 4% PFA and IgG was immunostained. Cell nuclei and plasma membranes were stained by DAPI and CellMask (ThermoFischer #H32720), respectively. Intracellular (defined by CellMask staining) ASO and IgG intensities were quantified using a customized workflow in Cell Profiler (Figurers 30 and 31). The results indicate that the two C-terminally linked <LNA3> binders can hold the fluorescent-linked Atto647N ASO, and depending on the N-terminal binder the antibody can either prevent (<nada>) or drive the uptake (hTfR low, hTfR high) of ASO. Object-based colocalization analysis revealed that premixing Atto647N-ASO with antibodies with a hTfR binder significantly increased the colocalization of Atto647N-ASO with transferrin receptor (FIG. 32), further indicating that the uptake of ASO is driven by the antibody.

### Example 11

### 3D Spheroid Assay

To assess the transcytosis efficiency of the antibodies and conjugates according to the current invention, a 3D spheroid assay was conducted (see, e.g., Simonneau et al., Fluids Barriers CNS 18 (2021) 43; Kassianidou and Simonneau et al., Bio Protoc 12 (2022) 4399). In more detail, primary human astrocytes (HA, ScienCell Research Laboratories), human brain microvascular pericytes (HBVP, ScienCell Research Laboratories), and human cerebral microvascular endothelial cells (hCMEC/D3, Merck) were maintained separately in the respective culture media. To generate the spheroids, HA, HBVP and hCMEC/D3 cells were resuspended at the appropriate concentration to target 1000 cells per microwell (600 μm in diameter and 720 μm in depth imprinted in polyethylene glycol (PEG) hydrogels (GRI3D® 96-well plate, SunBioscience)) in a 1:1:1 ratio in a seeding volume of 60 μL per well. 150 μL of media was added after 20 min. The cells were grown in a humidified incubator at 37° C. with 5% CO₂ for 48 h (with a medium refresh after 24 h) to allow self-assembly of the multicellular spheroids. After 48 h of assembly, BBB spheroid arrays were incubated with the tested molecules in media for 4 h at 37° C. with 5% CO₂. After incubation, BBB spheroids were washed and fixed in 4% PFA. Samples were permeabilized with 0.6% Triton-X and 10% donkey serum in DPBS for 1 h at RT. Anti-human FcY (H+L) and IgG were stained (Jackson ImmunoResearch 709-545-098; 488 fluorescently labelled). Finally, the samples were washed again, transferred to cover glasses, and mounted with Fluoromount (Electron Microscopy Science). Spheroids were imaged using a Leica Microsystems, Thunder Imager 3D Assay with a 20×/0.55 Ph2 dry objective. The images were acquired with a 2×2 binning in a 16 bit format. A z-stack covering a total depth of 8.5 μm, using 8 steps with the core placed at the center (1.21 μm step size) were used. At least 10 spheroids per condition per experiment were acquired. The Instant Computational Clearing (ICC) algorithm by Leica was then applied to the images. Analysis of images were performed using a customized code. Fluorescence intensity is reported per μm², by dividing raw integrated density over area (m²).

In the first set of experiments (FIG. 33), the <hTfR>—ASO conjugate largely increased the fluorescence intensity within the spheroid, suggesting the ASO payload caused non-specific uptake and transcytosis of conjugates into the spheroids. However, pre-incubating of the <hTfR>—ASO conjugate with 10× molar ratio of <LNA3> Fab, but not 10× molar ratio of DP47 Fab, decreased the fluorescence intensity within the spheroid. This result indicates that the LNA-binder can reduce large aggregates caused by the ASO payload on <hTfR>—ASO conjugates.

In a further set of experiments (FIG. 34), <hTfR>—ASO conjugates generated via FORCE technology were tested in wild-type or human TfR knock-out spheroids. The human TfR knock-out spheroids were used to access transferrin receptor independent transcytosis. The format with a C-terminally linked <LNA3> binder was selected, as this format predominantly forms monomers in the analytical size exclusion column. The construct with a covalently-linked <LNA3> binder, but not with <DP47>, significantly reduced fluorescence intensity within both the wild-type and human transferrin receptor knock-out spheroids. Therefore, a C-terminally linked <LNA3> binder can reduce the transferrin receptor independent uptake and transcytosis of <hTfR>—ASO conjugates.

In a further set of experiments, antibody-ASO conjugates generated by the conventional recombinant method were tested in spheroids (FIG. 35). The results again confirmed that a C-terminally linked <LNA3> binder can reduce non-specific uptake and transcytosis driven by the ASO payload.

### Example 12

### Primary Human Liver Sinusoidal Endothelial Cell Assay

To access the non-specific uptake of antibody-ASO conjugate, the Large molecule Unspecific Clearance Assay (LUCA) was used (see WO 2021/204743). The LUCA assay uses primary human liver endothelial cells. Data is acquired by labeling the antigen binding molecules with a pH-sensitive dye exhibiting high fluorescence, when accumulating in the lysosome (acidic pH 5.5) and low fluorescence when remaining outside the cell (neutral pH 7.4). The antibodies were labeled using the SiteClick™ Antibody Azido Modification Kit (Thermo Fisher Scientific) according to the manufacturer's instructions. Human or animal endothelial cells are incubated with labeled antibodies for 2 and 4 hours and the fluorescent readout is recorded using a flow cytometer. The geo-mean intensities are used for linear regression analysis. The extracted slopes form, when normalized to standard antibodies, the so-called relative LUCA rate.

In the first set of experiments (FIG. 36), <hTfR>only, <hTfR>—ASO conjugate, and <hTfR>—ASO conjugate pre-incubated with 10× molar ratio of the <LNA3> Fab were tested in the LUCA setting. At both time points (2 h and 4 h) the ASO-payload increased the normalized cellular accumulation, contributed by non-specific uptake and TMDD (target-mediated drug disposition). However, pre-incubation of 10× molar ratio of the <LNA3> Fab decreased the cellular accumulation of <hTfR>—ASO conjugate.

In a further set of experiments (FIG. 37), ASO conjugated to antibodies with an N-terminal <nada> non-binder and a C-terminally linked <DP47> (non-binder) or <LNA3> were tested in the LUCA setting. The relative LUCA rate (unspecific uptake) was decreased in the construct where ASO was conjugated to antibody with a C-terminally linked <LNA3>, as compared to ASO conjugated to a C-terminally linked <DP47>. Pre-incubating the <nada/LNA3>—ASO conjugate with 10× molar ratio of the <LNA3> Fab did not further reduce the relative LUCA rate, suggesting that one C-terminally linked <LNA3> is sufficient to fully reduce the unspecific uptake caused by the ASO payload.

In a further set of experiments (FIG. 38), the antibody with a N-terminal <nada> and two C-terminally linked <LNA3> binding sites with or without pre-incubation of 1× molar ratio of free ASO were tested in the LUCA setting. The two C-terminally linked <LNA3> binding sites can hold one ASO with high avidity (confirmed by SPR). Addition of non-covalently linked ASO to the construct did not increase the relative LUCA rate, suggesting the ASO is protected by the two <LNA3> binders.

### Example 13

### In Vivo Plasma Pharmacokinetics Study in Mice

To investigate the pharmacokinetics (PK) properties of antibody-ASO complexes and conjugates, an in vivo single-dose plasma PK study was conducted in male C57BL/6J mice. The animal grouping and dosing details are summarized in Table 8-1.

TABLE 8-1
Animal grouping and dose levels

| Group | Test Item | Dose [mg/kg] |
|---|---|---|
| 1 | ASO 827 | 0.93 |
| 2 | mAb 1927 | 40.9 |
| | ASO 827 | 0.93 |
| 3 | fusion 9545 | 21.3 |
| 4 | fusion 9544 | 21.5 |
| 5 | fusion 9543 | 21.5 |
| 6 | fusion 9542 | 21.6 |
| 7 | bsAb 1319 | 27.1 |
| | ASO 827 | 0.93 |
| 8 | bsAb 1926 | 27.1 |
| | ASO 827 | 0.93 |

Mice were administered intravenously (i.v.) with compounds (dose volume 5 mL/kg) listed in Table 8-1, including naked ASO (Group 1), ASO pre-incubated with a 3× molar ratio of IgG with one N-terminal anti-LNA antibody Fab fragment of the anti-LNA antibody produced by clone 1.9.21 (<LNA3> binding site; Group 2), ASO pre-incubated with a 1× molar ratio of bispecific antibodies with two C-terminally linked <LNA3> binding sites (Groups 7 and 8), or bispecific antibodies conjugated with ASO using the KTG technology (Groups 3, 4, 5, and 6). The conjugates were formulated in PBS with 250 mM arginine, pH 7.4, while antibodies were formulated in PBS, pH 7.4. For groups 2, 7, and 8, the ASO and antibody components were mixed one day before dosing. The ASO dose was consistent across all groups (equivalent to 0.93 mg/kg of ASO 827) to enable comparison of ASO plasma PK between groups.

Blood samples were collected at 10 min., 30 min., 6 hours, 24 hours, 72 hours and 168 hours (terminal) post-dosing into K3-EDTA-coated Minivette POCT (SARSTEDT AG & Co. KG, Nümbrecht, Germany), then transferred to 0.2 mL tubes and centrifuged at 4° C. at 10,000 g for approximately 5 min. Plasma samples were stored at −80° C. for subsequent analysis. Mice were euthanized 168 hours post-dosing by perfusion with PBS and heparin (16 UI/ml) under deep anesthesia with pentobarbital (60 mg/kg intraperitoneal injection). Brain tissues (cortex, cerebellum, rest of brain) were collected after termination.

ASO levels in plasma samples were quantified using a hybridization enzyme-linked immunosorbent assay (hELISA). Quality controls and plasma samples were prepared at double concentration (2×) using assay buffer (750 mM NaCl, 75 mM sodium citrate, 0.05% Tween 20, pH 7.0) with 1% mouse serum in a pre-dilution plate. The capture oligonucleotide probe labeled with biotin and the detection oligonucleotide probe labeled with digoxigenin were also prepared at 2× concentration before mixing with pre-diluted standards, quality controls, and plasma samples, followed by heating at 95° C. for 10 min. and cooling to room temperature (RT). Hybridized complexes were transferred to a streptavidin-coated microtiter plate (Microcoat Biotechnologie, Bernried, Germany) and incubated at RT for 1 hour with gentle shaking. After washing, the detection antibody (anti-digoxigenin Fab conjugated to POD; #11633716001; Roche Diagnostics GmbH, Mannheim, Germany) was added and incubated for 1 hour with gentle shaking. Visualization of the immobilized hybridized complexes was achieved by adding BM Blue (TMB; Roche Diagnostics GmbH, Mannheim, Germany) solution, with the optical density (OD) measured at 680 nm (reference wavelength 450 nm) under gentle shaking until a maximum of 0.7 OD was reached. The reaction was stopped by adding 50 μL of 1 M H2SO4, causing the products to turn yellow. Endpoint measurement was performed at 450 nm (reference 690 nm), with the highest standard reaching a maximum of 2.2 OD. ASO quantification was performed by back-calculating the OD values using a non-linear 4-parameter Rodbard-205 curve fitting function, with the standard calibration curve (naked ASO) prepared in assay buffer.

The results of this study revealed that the addition of <LNA> to unconjugated as well as conjugated ASO had an profound impact on the PK of ASO (FIGS. 39 and 40). Half-life extension was observed for non-covalently attached Fab and bsAb complexes (group 2 & 7 & 8), as well as for ASO-conjugates that harbored an additional <LNA> binding site (group 4 & 6) compared to controls that did not contain a <LNA> binding site.

### Example 14

### In Vivo Brain Exposure Study in Mice

To investigate the brain exposure of antibody-ASO complexes and conjugates, an in vivo single-dose plasma PK study was extended at termination point to assess brain exposure in male C57BL/6J mice. The animal grouping and dosing details are summarized in Table 8-1 of Example 13. Mice were administered intravenously (i.v.) with compounds (dose volume 5 mL/kg) listed in Table 8-1, including naked ASO (Group 1), ASO pre-incubated with a 3× molar ratio of IgG with one N-terminal anti-LNA antibody Fab fragment of the anti-LNA antibody produced by clone 1.9.21 (<LNA3> binding site; Group 2), ASO pre-incubated with a 1× molar ratio of bispecific antibodies with two C-terminally linked <LNA3> binding sites (Groups 7 and 8), or bispecific antibodies conjugated with ASO using the KTG technology (Groups 3, 4, 5, and 6). The conjugates were formulated in PBS with 250 mM arginine, pH 7.4, while antibodies were formulated in PBS, pH 7.4. For groups 2, 7, and 8, the ASO and antibody components were mixed one day before dosing. The ASO dose was consistent across all groups (equivalent to 0.93 mg/kg of ASO 827) to enable comparison of ASO plasma PK and brain exposure between groups.

Blood samples were collected at 10 min, 30 min, 6 hours, 24 hours, 72 hours and 168 hours (terminal) post-dosing into K3-EDTA-coated Minivette POCT (SARSTEDT AG & Co. KG, Nümbrecht, Germany), then transferred to 0.2 mL tubes and centrifuged at 4° C. at 10,000 g for approximately 5 min. Plasma samples were stored at −80° C. for subsequent analysis. Mice were euthanized 168 hours post-dosing by perfusion with PBS and heparin (16 UI/ml) under deep anesthesia with pentobarbital (60 mg/kg intraperitoneal injection). Brain tissues (cortex, cerebellum, rest of brain) were collected after termination.

Brain tissues were homogenized in MagNA Pure buffer (Roche Diagnostics GmbH, Mannheim, Germany; #06374913001) using 5 mm pre-cooled stainless steel beads (Qiagen, Hilden, Germany; #69989) on a Tissue Lyser II (Qiagen, Hilden, Germany; No. 853000) for 3 min. at 30 Hz. ASO levels in brain tissues were analyzed using the hELISA method as described in Example 13, except that quality controls and samples were diluted in assay buffer and hybridization with probes was performed at RT for 1 hour with gentle shaking.

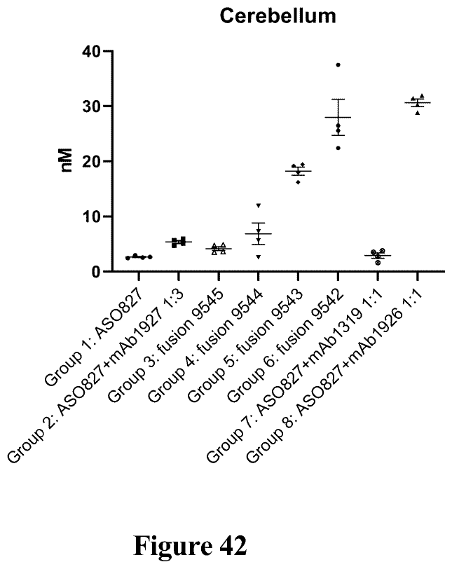

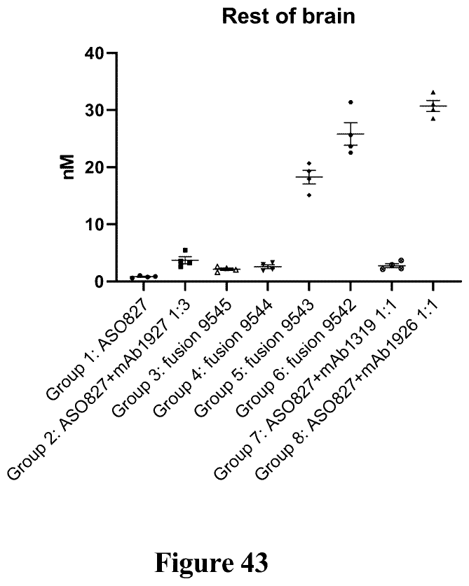

The results of this study revealed that the addition of <LNA> binding sites to unconjugated as well as conjugated ASO had an profound impact on the levels of ASO detectable in the brain (FIGS. 41 to 43). Increased ASO levels were observed in cortex, cerebellum and the rest of the brain for ASO which was non-covalently attached to a TfR-targeting <TfR><LNA> bsAb (group 8) compared to controls that did not contain <LNA> binding sites. Increased ASO levels were also observed in cortex, cerebellum and the rest of the brain for ASO which was covalently conjugated to a TfR-targeting <TfR><LNA> bsAb (group 6) compared to controls that did not contain <LNA> binding sites.

1. An anti-LNA antibody comprising
(a) HVR-H1 comprising the amino acid sequence of SEQ ID NO: 42;
(b) HVR-H2 comprising the amino acid sequence of SEQ ID NO: 44;
(c) HVR-H3 comprising the amino acid sequence of SEQ ID NO: 46;
(d) HVR-L1 comprising the amino acid sequence of SEQ ID NO: 51;
(e) HVR-L2 comprising the amino acid sequence of SEQ ID NO: 53; and
(f) HVR-L3 comprising the amino acid sequence of SEQ ID NO: 55.

2. The anti-LNA antibody according to claim 1 comprising a VH of SEQ ID NO: 48 and a VL of SEQ ID NO: 57.

3. The anti-LNA antibody according to claim 1, wherein the antibody is an antibody fragment selected from the group of antibody fragments consisting of Fv, scFv, Fab and scFab.

4. A multispecific antibody comprising
at least one first binding site specifically binding to LNA and comprising
(a) HVR-H1 comprising the amino acid sequence of SEQ ID NO: 42;
(b) HVR-H2 comprising the amino acid sequence of SEQ ID NO: 44;
(c) HVR-H3 comprising the amino acid sequence of SEQ ID NO: 46;
(d) HVR-L1 comprising the amino acid sequence of SEQ ID NO: 51;
(e) HVR-L2 comprising the amino acid sequence of SEQ ID NO: 53; and
(f) HVR-L3 comprising the amino acid sequence of SEQ ID NO: 55;
at least one second binding site specifically binding to a second non-LNA target,
an Fc-region comprising a first Fc-region polypeptide and a second Fc-region polypeptide,
wherein the at least one binding site specifically binding to LNA is conjugated to the C-terminus of one of the Fc-region polypeptides and the at least one binding site specifically binding to a second non-LNA target is conjugated to the N-terminus of one of the Fc-region polypeptides.

5. The multispecific antibody according to claim 4, wherein the antibody comprises
a) one first binding site and one second binding site, whereby both binding sites are conjugated to the same Fc-region polypeptide;
b) one first binding site and one second binding site, whereby both binding sites are conjugated to different Fc-region polypeptides;
c) two first binding sites each comprising the HVRs of SEQ ID NO: 42, 44, 46, 51, 53 and 55, one second binding site, whereby the C-terminus of each Fc-region polypeptide is conjugated to a single first binding site; or
d) two first binding sites each comprising the HVRs of SEQ ID NO: 42, 44, 46, 51, 53 and 55, and two second binding sites, whereby the N-terminus of each Fc-region polypeptide is conjugated to a single second binding site and the C-terminus of each Fc-region polypeptide is conjugated to a single first binding site.

6. The multispecific antibody according to claim 4, wherein each binding site is independently of each other an antibody fragment selected from the group of antibody fragments consisting of Fv, scFv, Fab and scFab.

7. The multispecific antibody according to claim 4, wherein the at least one first binding site comprises a VH of SEQ ID NO: 48 and a VL of SEQ ID NO: 57.

8. The multispecific antibody according to claim 4, wherein the at least one first binding site is conjugated to the C-terminus of the respective Fc-region polypeptide by a peptidic linker, and wherein the peptidic linker is a GS-linker comprising GGGS (SEQ ID NO: 148) or GGGGS (SEQ ID NO: 149) elements and a total number of amino acid residues in the range of and including 20 amino acid residues to 40 amino acid residues.

9. The anti-LNA antibody according to claim 1, wherein the antibody comprises
an Fc-region comprising a first Fc-region polypeptide and a second Fc-region polypeptide, and
at least one recognition site(s) for the transglutaminase from Kutzneria albida (KalbTG) inserted in one or both of the Fc-region polypeptides.

10. The anti-LNA antibody or the multispecific antibody according to claim 9, wherein either one recognition site or one recognition site in each Fc-region polypeptide is(are) inserted directly after position 297 of the Fc-region polypeptide (numbering according to Kabat).

11. The anti-LNA antibody or the multispecific antibody according to claim 9, wherein each of the at least one recognition sites has the sequence of SEQ ID NO: 134 or of SEQ ID NO: 143, in case of more than one recognition site independently of each other.

12. The anti-LNA antibody or the multispecific antibody according to claim 9, wherein the antibody is conjugated to a payload via the at least one recognition site using KTG.

13. The anti-LNA antibody or the multispecific antibody according to claim 12, wherein the payload is selected from a small molecule, a peptide or polypeptide, a dye, a nucleic acid, an siRNA, an antisense oligonucleotide and an LNA.


SEQUENCE LISTING (150 sequences)

SEQ ID NO: 1 - DNA, 14 bp, synthetic construct
  source 1..14: mol_type = other DNA
gtctctttacctgg

SEQ ID NO: 2 - DNA, 13 bp, synthetic construct
  source 1..13: mol_type = other DNA
actccaaatcctg

SEQ ID NO: 3 - DNA, 14 bp, synthetic construct
  source 1..14: mol_type = other DNA
aagtctgtttcacc

SEQ ID NO: 4 - protein, 19 aa, Mus musculus
MAVLGLLFCLVTFPSCVLS

SEQ ID NO: 5 - protein, 30 aa, Mus musculus
QVQLKQSGPSFVQPSQSLSITCTVSGFSLT

SEQ ID NO: 6 - protein, 5 aa, Mus musculus
TYGVH

SEQ ID NO: 7 - protein, 14 aa, Mus musculus
WVRQSPGKGLEWLG

SEQ ID NO: 8 - protein, 16 aa, Mus musculus
VIWRSGGTDYNAAFMS

SEQ ID NO: 9 - protein, 32 aa, Mus musculus
RLSITKDNSKSQVFFKMNSLQTDDTAIYYCTN

SEQ ID NO: 10 - skipped

SEQ ID NO: 11 - protein, 11 aa, Mus musculus
WGQGTTLTVSS

SEQ ID NO: 12 - protein, 111 aa, Mus musculus
QVQLKQSGPSFVQPSQSLSITCTVSGFSLTTYGVHWVRQSPGKGLEWLGVIWRSGGTDYNAAFMSRLSITKDNSKSQVFFKMNSLQTDDTAIYYCTNLYYWGQGTTLTVSS

SEQ ID NO: 13 - protein, 20 aa, Mus musculus
MSVPTQVLGLLVLWIPGSTA

SEQ ID NO: 14 - protein, 23 aa, Mus musculus
DIVMTQATFSNPVTLGTSVSISC

SEQ ID NO: 15 - protein, 16 aa, Mus musculus
RSSKSLLRSNGITYLY

SEQ ID NO: 16 - protein, 15 aa, Mus musculus
WYLQKPGQSPQLLIY

SEQ ID NO: 17 - protein, 7 aa, Mus musculus
QMSNLAS

SEQ ID NO: 18 - protein, 32 aa, Mus musculus
GVPDRFSSSGSGTDFTLRISRVEAEDVGVYYC

SEQ ID NO: 19 - protein, 9 aa, Mus musculus
GQNLELPYT

SEQ ID NO: 20 - protein, 10 aa, Mus musculus
FGSGTKVEIK

SEQ ID NO: 21 - protein, 112 aa, Mus musculus
DIVMTQATFSNPVTLGTSVSISCRSSKSLLRSNGITYLYWYLQKPGQSPQLLIYQMSNLASGVPDRFSSSGSGTDFTLRISRVEAEDVGVYYCGQNLELPYTFGSGTKVEIK

SEQ ID NO: 22 - protein, 19 aa, Mus musculus
MAVLGLFFCLVAFPSCVLS

SEQ ID NO: 23 - protein, 30 aa, Mus musculus
QIQLRESGPGLVAPSQSLSITCTVSGFSLT

SEQ ID NO: 24 - protein, 5 aa, Mus musculus
NYGVH

SEQ ID NO: 25 - protein, 14 aa, Mus musculus
WVRQPPGKGLEWLG

SEQ ID NO: 26 - protein, 16 aa, Mus musculus
VIWAGGSTIYNSALMS

SEQ ID NO: 27 - protein, 31 aa, Mus musculus
RLSISRDNSKSQVFLEMNSLQTYDTAMYYCA

SEQ ID NO: 28 - protein, 12 aa, Mus musculus
RDRPFDGFFFSY

SEQ ID NO: 29 - protein, 11 aa, Mus musculus
WGQGTALTVSS

SEQ ID NO: 30 - protein, 119 aa, Mus musculus
QIQLRESGPGLVAPSQSLSITCTVSGFSLTNYGVHWVRQPPGKGLEWLGVIWAGGSTIYNSALMSRLSISRDNSKSQVFLEMNSLQTYDTAMYYCARDRPFDGFFFSYWGQGTALTVSS

SEQ ID NO: 31 - protein, 19 aa, Mus musculus
MKLPVRLLVLMFWIPACNS

SEQ ID NO: 32 - protein, 23 aa, Mus musculus
DVLMTQTPLSLPVSLGDQASISC

SEQ ID NO: 33 - protein, 16 aa, Mus musculus
RSSQAIVHRSGNTFLE

SEQ ID NO: 34 - protein, 15 aa, Mus musculus
WYLQKPGQSPKLLIY

SEQ ID NO: 35 - protein, 7 aa, Mus musculus
KVSNRFS

SEQ ID NO: 36 - protein, 32 aa, Mus musculus
GVPDRFSGSGSGTDFTLKINRVEAEDLGVYYC

SEQ ID NO: 37 - protein, 9 aa, Mus musculus
FQGSHVPYT

SEQ ID NO: 38 - protein, 10 aa, Mus musculus
FGSGTKLEIK

SEQ ID NO: 39 - protein, 112 aa, Mus musculus
DVLMTQTPLSLPVSLGDQASISCRSSQAIVHRSGNTFLEWYLQKPGQSPKLLIYKVSNRFSGVPDRFSGSGSGTDFTLKINRVEAEDLGVYYCFQGSHVPYTFGSGTKLEIK

SEQ ID NO: 40 - protein, 19 aa, Mus musculus
MEWSWVFLFLLSVTAGVHS

SEQ ID NO: 41 - protein, 30 aa, Mus musculus
QVQLQQSGAELVRPGTSVKVSCKASGYAFT

SEQ ID NO: 42 - protein, 5 aa, Mus musculus
NYLIE

SEQ ID NO: 43 - protein, 14 aa, Mus musculus
WIKQRPGQGLEWIG

SEQ ID NO: 44 - protein, 17 aa, Mus musculus
VINPGSGGTNYNEKFRV

SEQ ID NO: 45 - protein, 32 aa, Mus musculus
KATLTADKSSSTAYMQLSSLTSDDSAVYFCAR

SEQ ID NO: 46 - protein, 5 aa, Mus musculus
GGGYY

SEQ ID NO: 47 - protein, 11 aa, Mus musculus
WGQGTLVTVSA

SEQ ID NO: 48 - protein, 114 aa, Mus musculus
QVQLQQSGAELVRPGTSVKVSCKASGYAFTNYLIEWIKQRPGQGLEWIGVINPGSGGTNYNEKFRVKATLTADKSSSTAYMQLSSLTSDDSAVYFCARGGGYYWGQGTLVTVSA

SEQ ID NO: 49 - protein, 20 aa, Mus musculus
MVSSAQFLGLLVLWIPGAIG

SEQ ID NO: 50 - protein, 23 aa, Mus musculus
DIVMTQAAPSVPVTPGESVSISC

SEQ ID NO: 51 - protein, 16 aa, Mus musculus
RSSKSLLHSNGNTYLF

SEQ ID NO: 52 - protein, 15 aa, Mus musculus
WFLQRPGQSPQVLIY

SEQ ID NO: 53 - protein, 7 aa, Mus musculus
RMSNLAS

SEQ ID NO: 54 - protein, 32 aa, Mus musculus
GVPDRFSGSGSGTAFTLRISRVEAEDVGVYYC

SEQ ID NO: 55 - protein, 9 aa, Mus musculus
MQHLEYPYT

SEQ ID NO: 56 - protein, 10 aa, Mus musculus
FGSGTRLEIK

SEQ ID NO: 57 - protein, 112 aa, Mus musculus
DIVMTQAAPSVPVTPGESVSISCRSSKSLLHSNGNTYLFWFLQRPGQSPQVLIYRMSNLASGVPDRFSGSGSGTAFTLRISRVEAEDVGVYYCMQHLEYPYTFGSGTRLEIK

SEQ ID NO: 58 - protein, 30 aa, Mus musculus
QIQLRESGPGLVAPSQSLSITCTVSGFSLV

SEQ ID NO: 59 - protein, 5 aa, Mus musculus
NYGIH

SEQ ID NO: 60 - protein, 14 aa, Mus musculus
WVRQPPGKGLEWLG

SEQ ID NO: 61 - protein, 16 aa, Mus musculus
VIWAGGSTIYNSALMS

SEQ ID NO: 62 - protein, 32 aa, Mus musculus
RLSISRDNSKNQVFLEMNSLQTYDTAMYYCAR

SEQ ID NO: 63 - protein, 11 aa, Mus musculus
DRPFDGFFFSY

SEQ ID NO: 64 - protein, 11 aa, Mus musculus
WGQGTALTVSS

SEQ ID NO: 65 - protein, 119 aa, Mus musculus
QIQLRESGPGLVAPSQSLSITCTVSGFSLVNYGIHWVRQPPGKGLEWLGVIWAGGSTIYNSALMSRLSISRDNSKNQVFLEMNSLQTYDTAMYYCARDRPFDGFFFSYWGQGTALTVSS

SEQ ID NO: 66 - protein, 23 aa, Mus musculus
DVLMTQTPLSLPVSLGDQASISC

SEQ ID NO: 67 - protein, 16 aa, Mus musculus
RSSQAIVHRSGNTFLE

SEQ ID NO: 68 - protein, 15 aa, Mus musculus
WYLQKPGQSPKLLIY

SEQ ID NO: 69 - protein, 7 aa, Mus musculus
KVSNRFS

SEQ ID NO: 70 - protein, 32 aa, Mus musculus; identical to SEQ ID NO: 36

SEQ ID NO: 71 - protein, 9 aa, Mus musculus
FQGSHVPYT

SEQ ID NO: 72 - protein, 10 aa, Mus musculus
FGSGTKLEIK

SEQ ID NO: 73 - protein, 112 aa, Mus musculus; identical to SEQ ID NO: 39

SEQ ID NO: 74 - protein, 30 aa, Mus musculus
QVQLKESGPGLVAPSQSLSITCTISGFSLT

SEQ ID NO: 75 - protein, 5 aa, Mus musculus
TYGVH

SEQ ID NO: 76 - protein, 14 aa, Mus musculus
WVRQPPGMGLEWLV

SEQ ID NO: 77 - protein, 16 aa, Mus musculus
VIWGDGSTTYNSALRS

SEQ ID NO: 78 - protein, 32 aa, Mus musculus
RLSISKDNSRSQVFLKMNSLQTDDTAMYYCAR

SEQ ID NO: 79 - protein, 9 aa, Mus musculus
HGGRYALDY

SEQ ID NO: 80 - protein, 11 aa, Mus musculus
WGQGTSVTVSS

SEQ ID NO: 81 - protein, 117 aa, Mus musculus
QVQLKESGPGLVAPSQSLSITCTISGFSLTTYGVHWVRQPPGMGLEWLVVIWGDGSTTYNSALRSRLSISKDNSRSQVFLKMNSLQTDDTAMYYCARHGGRYALDYWGQGTSVTVSS

SEQ ID NO: 82 - protein, 23 aa, Mus musculus
DIVMTQSPATLSVTPGDRVSLSC

SEQ ID NO: 83 - protein, 11 aa, Mus musculus
RASQSIGGYLH

SEQ ID NO: 84 - protein, 15 aa, Mus musculus
WYQQKSHESPRLLIK

SEQ ID NO: 85 - protein, 7 aa, Mus musculus
YASQSMS

SEQ ID NO: 86 - protein, 32 aa, Mus musculus
GIPPRFSGSGSGSDFTLSINSVAPEDVGVYYC

SEQ ID NO: 87 - protein, 9 aa, Mus musculus
QAGHTFPLT

SEQ ID NO: 88 - protein, 7 aa, Mus musculus
FGAGTKL

SEQ ID NO: 89 - protein, 104 aa, Mus musculus
DIVMTQSPATLSVTPGDRVSLSCRASQSIGGYLHWYQQKSHESPRLLIKYASQSMSGIPPRFSGSGSGSDFTLSINSVAPEDVGVYYCQAGHTFPLTFGAGTKL

SEQ ID NO: 90 - protein, 225 aa, Mus musculus
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 91 - protein, 227 aa, Mus musculus
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 92 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 93 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 94 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 95 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 96 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 97 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 98 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 99 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 100 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 101 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 102 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 103 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 104 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 105 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLSCAVKGFYPSDIAVEWESNGQPENNYKTTPPVLDSDGSFFLVSRLTVDKSRWQEGNVFSCSVMHEALHNHYTQKSLSLSL

SEQ ID NO: 106 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 107 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSP

SEQ ID NO: 108 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 109 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSP

SEQ ID NO: 110 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 111 - protein, 225 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNAYTQKSLSLSP

SEQ ID NO: 112 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 113 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 114 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 116 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 117 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 118 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALANHATQKSLSLSP

SEQ ID NO: 119 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALANHATQKSLSLSP

SEQ ID NO: 120 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALANHATQKSLSLSP

SEQ ID NO: 121 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALANHATQKSLSLSP

SEQ ID NO: 122 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALANHATQKSLSLSP

SEQ ID NO: 123 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALANHATQKSLSLSP

SEQ ID NO: 124 - DNA, 14 bp, synthetic construct
  source 1..14: mol_type = other DNA
gtctctttacctgg

SEQ ID NO: 125 - DNA, 13 bp, synthetic construct
  source 1..13: mol_type = other DNA
actccaaatcctg

SEQ ID NO: 126 - DNA, 14 bp, synthetic construct
  source 1..14: mol_type = other DNA
aagtctgtttcacc

SEQ ID NO: 127 - DNA, 20 bp, synthetic construct
  source 1..20: mol_type = other DNA
  modified_base 17: mod_base = OTHER; note = 5-methyl cytosine
  modified_base 19: mod_base = OTHER; note = 5-methyl cytosine
  modified_base 1..4: mod_base = OTHER; note = beta-D-oxy-LNA nucleoside
  modified_base 17..20: mod_base = OTHER; note = beta-D-oxy-LNA nucleoside
  modified_base 1..19: mod_base = OTHER; note = phosphorothioate backbone
  modified_base 1: mod_base = OTHER; note = conjugated to BCN_2 via phosphorothioate linkage
ttaactcaaatcaattctca

SEQ ID NO: 128 - DNA, 20 bp, synthetic construct
  misc_feature 1..19: note = RNA
  misc_feature 20: note = DNA
  source 1..20: mol_type = other DNA
  misc_feature 2: note = 2'-O-methyl ribose
  misc_feature 4: note = 2'-O-methyl ribose
  misc_feature 6: note = 2'-O-methyl ribose
  misc_feature 8: note = 2'-O-methyl ribose
  misc_feature 10: note = 2'-O-methyl ribose
  misc_feature 12: note = 2'-O-methyl ribose
  misc_feature 14: note = 2'-O-methyl ribose
  misc_feature 16: note = 2'-O-methyl ribose
  misc_feature 18: note = 2'-O-methyl ribose
  misc_feature 1..2: note = phosphorothioate backbone
  misc_feature 18..19: note = phosphorothioate backbone
  misc_feature 1: note = 2'-fluoro ribose
  misc_feature 3: note = 2'-fluoro ribose
  misc_feature 5: note = 2'-fluoro ribose
  misc_feature 7: note = 2'-fluoro ribose
  misc_feature 9: note = 2'-fluoro ribose
  misc_feature 11: note = 2'-fluoro ribose
  misc_feature 13: note = 2'-fluoro ribose
  misc_feature 15: note = 2'-fluoro ribose
  misc_feature 17: note = 2'-fluoro ribose
  misc_feature 19: note = 2'-fluoro ribose
  misc_feature 20: note = inverted DNA
tctcgtggccttaatgaaat

SEQ ID NO: 129 - RNA, 21 nt, synthetic construct
  source 1..21: mol_type = other RNA
  misc_feature 2: note = 2'-fluoro ribose
  misc_feature 4: note = 2'-fluoro ribose
  misc_feature 6: note = 2'-fluoro ribose
  misc_feature 8: note = 2'-fluoro ribose
  misc_feature 10: note = 2'-fluoro ribose
  misc_feature 12: note = 2'-fluoro ribose
  misc_feature 14: note = 2'-fluoro ribose
  misc_feature 16: note = 2'-fluoro ribose
  misc_feature 18: note = 2'-fluoro ribose
  misc_feature 1: note = 2'-O-methyl ribose
  misc_feature 3: note = 2'-O-methyl ribose
  misc_feature 5: note = 2'-O-methyl ribose
  misc_feature 7: note = 2'-O-methyl ribose
  misc_feature 9: note = 2'-O-methyl ribose
  misc_feature 11: note = 2'-O-methyl ribose
  misc_feature 13: note = 2'-O-methyl ribose
  misc_feature 15: note = 2'-O-methyl ribose
  misc_feature 17: note = 2'-O-methyl ribose
  misc_feature 19: note = 2'-O-methyl ribose
  misc_feature 20: note = 2'-O-methyl ribose
  misc_feature 21: note = 2'-O-methyl ribose
  misc_feature 1..2: note = phosphorothioate backbone
  misc_feature 19..20: note = phosphorothioate backbone
tttcattaaggccacgagatt

SEQ ID NO: 130 - protein, 226 aa, Homo sapiens
QVQLQQSGAELVRPGTSVKVSCKASGYAFTNYLIEWIKQRPGQGLEWIGVINPGSGGTNYNEKFRVKATLTADKSSSTAYMQLSSLTSDDSAVYFCARGGGYYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEPEA

SEQ ID NO: 131 - protein, 219 aa, Homo sapiens
DIVMTQAAPSVPVTPGESVSISCRSSKSLLHSNGNTYLFWFLQRPGQSPQVLIYRMSNLASGVPDRFSGSGSGTAFTLRISRVEAEDVGVYYCMQHLEYPYTFGSGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - DNA, 19 bp, synthetic construct
  source 1..19: mol_type = other DNA
  modified_base 1: mod_base = OTHER; note = 5-methyl cytosine
  modified_base 15: mod_base = OTHER; note = 5-methyl cytosine
  modified_base 17: mod_base = OTHER; note = 5-methyl cytosine
  modified_base 1..4: mod_base = OTHER; note = beta-D-oxy-LNA nucleoside
  modified_base 15: mod_base = OTHER; note = beta-D-oxy-LNA nucleoside
  modified_base 17..19: mod_base = OTHER; note = beta-D-oxy-LNA nucleoside
  modified_base 1..18: mod_base = OTHER; note = phosphorothioate backbone
  modified_base 1: mod_base = OTHER; note = conjugated to BCN_2 via phosphorothioate linkage
ctttaatttaatcactcat

SEQ ID NO: 133 - skipped

SEQ ID NO: 134 - protein, 5 aa, synthetic construct
YRYRQ

SEQ ID NO: 135 - DNA, 20 bp, synthetic construct
  source 1..20: mol_type = other DNA
  modified_base 17: mod_base = OTHER; note = 5-methyl cytosine
  modified_base 19: mod_base = OTHER; note = 5-methyl cytosine
  modified_base 1..4: mod_base = OTHER; note = beta-D-oxy-LNA nucleoside
  modified_base 17..20: mod_base = OTHER; note = beta-D-oxy-LNA nucleoside
  modified_base 1..19: mod_base = OTHER; note = phosphorothioate backbone
  modified_base 1: mod_base = OTHER; note = conjugated to A6 and BCN_PEG3 via phosphorothioate linkage
ttaactcaaatcaattctca

SEQ ID NO: 136 - protein, 5 aa, synthetic construct
RYESK

SEQ ID NO: 137 - protein, 225 aa, synthetic construct
EVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARAGHYDGGHLGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 138 - protein, 219 aa, synthetic construct
DVVMTQTPLSLPVSLGDQASISCRSSQSLVHSNGYTYLHWYLQKPGQSPKLLIYKVSNRFSGVPDRFSGSGSGTDFTLKISRVEADDLGVYFCSQSTRVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 20 bp, synthetic construct
  source 1..20: mol_type = other DNA
  modified_base 17: mod_base = OTHER; note = 5-methyl cytosine
  modified_base 19: mod_base = OTHER; note = 5-methyl cytosine
  modified_base 1..4: mod_base = OTHER; note = beta-D-oxy-LNA nucleoside
  modified_base 17..20: mod_base = OTHER; note = beta-D-oxy-LNA nucleoside
  modified_base 1..19: mod_base = OTHER; note = phosphorothioate backbone
ttaactcaaatcaattctca

SEQ ID NO: 140 - DNA, 19 bp, synthetic construct
  source 1..19: mol_type = other DNA
  modified_base 1: mod_base = OTHER; note = 5-methyl cytosine
  modified_base 15: mod_base = OTHER; note = 5-methyl cytosine
  modified_base 17: mod_base = OTHER; note = 5-methyl cytosine
  modified_base 1..4: mod_base = OTHER; note = beta-D-oxy-LNA nucleoside
  modified_base 15: mod_base = OTHER; note = beta-D-oxy-LNA nucleoside
  modified_base 17..19: mod_base = OTHER; note = beta-D-oxy-LNA nucleoside
  modified_base 1..18: mod_base = OTHER; note = phosphorothioate backbone
ctttaatttaatcactcat

SEQ ID NO: 141 - DNA, 16 bp, synthetic construct
  source 1..16: mol_type = other DNA
  modified_base 15: mod_base = OTHER; note = 5-methyl cytosine
  modified_base 1..3: mod_base = OTHER; note = beta-D-oxy-LNA nucleoside
  modified_base 14..16: mod_base = OTHER; note = beta-D-oxy-LNA nucleoside
  modified_base 1..15: mod_base = OTHER; note = phosphorothioate backbone
gagttacttgccaact

SEQ ID NO: 142 - DNA, 18 bp, synthetic construct
  source 1..18: mol_type = other DNA
  modified_base 1: mod_base = OTHER; note = 5-methyl cytosine
  modified_base 6: mod_base = OTHER; note = 5-methyl cytosine
  modified_base 1: mod_base = OTHER; note = beta-D-oxy-LNA nucleoside
  modified_base 3: mod_base = OTHER; note = beta-D-oxy-LNA nucleoside
  modified_base 6: mod_base = OTHER; note = beta-D-oxy-LNA nucleoside
  modified_base 9: mod_base = OTHER; note = beta-D-oxy-LNA nucleoside
  modified_base 11: mod_base = OTHER; note = beta-D-oxy-LNA nucleoside
  modified_base 14: mod_base = OTHER; note = beta-D-oxy-LNA nucleoside
  modified_base 17..18: mod_base = OTHER; note = beta-D-oxy-LNA nucleoside
  modified_base 1..17: mod_base = OTHER; note = beta-D-oxy-LNA nucleoside
cctttcactcgtttccag

SEQ ID NO: 143 - protein, 13 aa, synthetic construct
GGGSYRYRQGGGS

SEQ ID NO: 144 - protein, 225 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 145 - protein, 217 aa, synthetic construct
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 221 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 147 - protein, 214 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 4 aa, synthetic construct
GGGS

SEQ ID NO: 149 - protein, 5 aa, synthetic construct
GGGGS

SEQ ID NO: 150 - DNA, 19 bp, synthetic construct
  source 1..19: mol_type = other DNA
  modified_base 1: mod_base = OTHER; note = 5-methyl cytosine
  modified_base 15: mod_base = OTHER; note = 5-methyl cytosine
  modified_base 17: mod_base = OTHER; note = 5-methyl cytosine
  modified_base 1..4: mod_base = OTHER; note = beta-D-oxy-LNA nucleoside
  modified_base 15: mod_base = OTHER; note = beta-D-oxy-LNA nucleoside
  modified_base 17..19: mod_base = OTHER; note = beta-D-oxy-LNA nucleoside
  modified_base 1..18: mod_base = OTHER; note = phosphorothioate backbone
  modified_base 1: mod_base = OTHER; note = conjugated to A6 and BCN_PEG3 via phosphothioate linkage
ctttaatttaatcactcat